(19) United States
(12) Patent Application Publication
(10) Pub. No.: US 2024/0350299 A1
(43) Pub. Date: Oct. 24, 2024

(54) SYSTEM AND METHOD FOR APPLYING THERAPEUTIC THERMAL CYCLES

(71) Applicant: Brain Tunnelgenix Technologies Corp., Aventura, FL (US)

(72) Inventor: Marcio Marc ABREU, Aventura, FL (US)

(73) Assignee: Brain Tunnelgenix Technologies Corp., Aventura, FL (US)

(21) Appl. No.: 18/636,075

(22) Filed: Apr. 15, 2024

Related U.S. Application Data
(60) Provisional application No. 63/496,385, filed on Apr. 14, 2023.

(51) Int. Cl. A61F 7/00

(52) U.S. Cl. CPC A61F 7/0053; A61F 7/007

(57) ABSTRACT

A system and method for applying temperature cycles to a patient for modulation, neutralization, or reduction of harmful molecules including inflammatory markers and cytokines as well as generation or increase of protective molecules and remodeling of molecules, including proteins. Such protective molecules can include heat shock proteins, also known by their initials as HSP, particularly heat shock proteins that are beneficial in combatting specific diseases and conditions. More specifically, the applied heat cycles from the temperature therapy systems disclosed herein help to generate heat shock proteins and reduce or normalize inflammatory molecules such as cytokines, for example, tumor necrosis factor alpha (TNF alpha biomarker), or interleukins, including but not limited to interleukin 2-6-13, and interleukin 17, and cancer-signaling molecules, including, but not limited to C-reactive proteins CA-19-9, CA-125, and CA 27-29. Manipulation of P53 protein for protection against and treatment of cancer, while eliminating toxic wastes is also possible.

### TECHNICAL FIELD

This disclosure relates to systems and methods for the treatment and diagnosis of various diseases that include controlling, modulating, and neutralizing harmful molecules including inflammatory markers and cytokines as well as generating or increasing protective molecules, including proteins, remodeling molecules including heat shock proteins (HSP), and neutralizing/reducing harmful molecules, particularly in human beings.

### BACKGROUND

Humans are known to have a variety of diseases and conditions. Traditional approaches to treating most diseases and conditions include surgery and medications.

### SUMMARY

This disclosure provides a system applying thermal therapy of a human being. The system comprises a chamber, a plurality of heaters, at least one heater power supply, a processor, and a support for the human. The chamber includes an upper wall, two side walls extending longitudinally in a direction parallel to the upper wall, a rear end wall, and a front wall that form an interior. The plurality of heaters is positioned in the interior of the chamber. The at least one heater power supply is connected to the plurality of heaters by a wire. The processor is connected to the at least one heater power supply to control an output of the at least one heater power supply to control heat output from the plurality of heaters according to at least one predetermined frequency, a predetermined maximum amplitude, and a predetermined minimum amplitude. The interior of the chamber is sized and dimensioned to contain a body of the human positioned on the support.

This disclosure also provides a heater system for applying thermal therapy to an Abreu Brain Thermal Tunnel (ABTT) of a human. The heater system comprises an inductor sized and dimensioned to contact the ABTT and at least one of a cooler, a heater, or a combination cooler and heater connected to the inductor and configured to provide heat to or a reduction of temperature of the ABTT according to at least one first predetermined frequency, a predetermined first maximum amplitude, and a predetermined first minimum amplitude.

This disclosure also provides a heater system for applying thermal therapy to an Abreu Brain Thermal Tunnel (ABTT) of a human, the heater system comprising a first air reservoir, a first heater, cooler, or combination heater and cooler, a first air nozzle, a first valve, a second air reservoir, a second heater, cooler, or combination heater and cooler, a second air nozzle, and a second valve. The first air reservoir is configured to contain compressed air. The first heater, cooler, or combination heater and cooler is attached to the first air reservoir and configured to provide heating and/or cooling to the compressed air in the first air reservoir at a first temperature. The first air nozzle is attached to the first air reservoir. The first air nozzle has a first end and a second end. The first valve is attached to the first air reservoir at a location between the first air reservoir and the first end of the first air nozzle. The second air reservoir is configured to contain compressed air. The second heater, cooler, or combination heater and cooler is attached to the second air reservoir and configured to provide heating and/or cooling to the compressed air in the first air reservoir at a second temperature lower than the first temperature. The second air nozzle is attached to the second air reservoir. The second air nozzle has a first end and a second end. The second valve is attached to the second air reservoir at a location between the second air reservoir and the first end of the second air nozzle. The first valve and the second valve are operated alternately for at least one predetermined frequency.

The advantages and features of the embodiments of this disclosure will become more apparent from the following detailed description of exemplary embodiments when viewed in conjunction with the accompanying drawings.

### BRIEF DESCRIPTION OF THE DRAWINGS

FIG. 1 shows a temperature therapy system in accordance with an exemplary embodiment of the present disclosure.

FIG. 2 shows a perspective view of a chamber of the temperature therapy system of FIG. 1.

FIG. 3 shows a first end or front view of the chamber of FIG. 2.

FIG. 4 shows a side view of the chamber of FIG. 2.

FIG. 5 shows a top view of the chamber of FIG. 2.

FIG. 6 shows a top plan view of a patient support of the temperature therapy system of FIG. 1.

FIG. 7 shows a partially exploded view of a front wall of the temperature therapy system of FIG. 1.

FIG. 8 shows a front view of the chamber of FIG. 1 with the front wall removed.

FIG. 9 shows a perspective view of a temperature therapy system in accordance with another exemplary embodiment of the present disclosure.

FIG. 10 shows a perspective view of a temperature therapy system in accordance with yet another exemplary embodiment of the present disclosure.

FIG. 11 shows a further perspective view of the temperature therapy system of FIG. 10.

FIG. 12 shows a perspective view of a patient support in accordance with an exemplary embodiment of the present disclosure.

FIG. 13 shows the patient support of FIG. 12 with both an internal patient support and an external patient support in a raised position.

FIG. 14 shows a perspective view of a temperature therapy system of the present disclosure in accordance with still another exemplary embodiment of the present disclosure.

FIG. 15 shows the perspective view of the temperature therapy system of FIG. 14 with a slider of a patient support positioned at an internal location of a chamber of the temperature therapy system.

FIG. 16 shows another perspective view of the temperature therapy system of FIG. 14 with an exemplary location for sensors in a chamber of the temperature therapy system.

FIG. 17 shows a perspective view of a temperature therapy system in accordance with yet still another exemplary embodiment of the present disclosure.

FIG. 18 shows a perspective view of a temperature therapy system in accordance with a further exemplary embodiment of the present disclosure.

FIG. 19A shows a perspective view of a partially sectioned temperature therapy system in accordance with a still further exemplary embodiment of the present disclosure.

FIG. 19B shows a further perspective view of the temperature therapy system of FIG. 19A with a patient in the chamber.

FIG. 20 shows a perspective view of a temperature therapy system in accordance with an exemplary embodiment of the present disclosure.

FIG. 21 shows a further perspective view of the temperature therapy system of FIG. 20.

FIG. 22 shows a front view of the temperature therapy system of FIG. 20.

FIG. 23 shows a back view of the temperature therapy system of FIG. 20.

FIG. 24 shows a side view of the temperature therapy system of FIG. 20.

FIG. 25 shows a top plan view of the temperature therapy system of FIG. 20.

FIG. 26 shows a bottom plan view of the temperature therapy system of FIG. 20.

FIG. 27 shows a perspective view of a smartwatch with a temperature inductor positioned thereon configured to provide heat to an Abreu brain thermal tunnel (ABTT) and to interface with the temperature therapy systems of the present disclosure in accordance with an exemplary embodiment of the present disclosure.

FIG. 28 shows a top view of the smart watch of FIG. 27.

FIG. 29 shows a side view of the smart watch of FIG. 27.

FIG. 30 shows a bottom view of the smart watch of FIG. 27.

FIG. 31 shows a perspective view of a hat, cap, or head covering with a temperature inductor positioned thereon configured to provide heat to a pair of Abreu brain thermal tunnels (ABTTs) and to interface with the temperature therapy systems of the present disclosure in accordance with another exemplary embodiment of the present disclosure.

FIG. 32 shows a front elevation view of the hat, cap, or head covering of FIG. 31.

FIG. 33 shows a side elevation view of the hat, cap, or head covering of FIG. 31.

FIG. 34 shows a bottom plan view of the hat, cap, or head covering of FIG. 31.

FIG. 35 shows a back elevation view of the hat, cap, or head covering of FIG. 31.

FIG. 36 shows a perspective view of a head covering with a temperature inductor positioned thereon to provide heat to a pair of Abreu brain thermal tunnels (ABTTs) and to interface with the temperature therapy systems of the present disclosure in accordance with a further exemplary embodiment of the present disclosure.

FIG. 37 shows a front elevation view of the head covering of FIG. 36.

FIG. 38 shows a back elevation view of the head covering of FIG. 36.

FIG. 39 shows a side elevation view of the head covering of FIG. 36.

FIG. 40 shows a top plan view of the head covering of FIG. 36.

FIG. 41 shows a bottom plan view of the head covering of FIG. 36.

FIG. 42 shows a perspective view of a head covering with a temperature inductor positioned thereon to provide heat to a pair of Abreu brain thermal tunnels (ABTTs) and to interface with the temperature therapy systems of the present disclosure in accordance with still another exemplary embodiment of the present disclosure.

FIG. 43 shows a front elevation view of the head covering of FIG. 42.

FIG. 44 shows a back elevation view of the head covering of FIG. 42.

FIG. 45 shows a side elevation view of the head covering of FIG. 42.

FIG. 46 shows a top plan view of the head covering of FIG. 42.

FIG. 47 shows a bottom plan view of the head covering of FIG. 42.

FIG. 48 shows a perspective view of a pair of glasses with a temperature inductor positioned thereon to provide heat to a pair of Abreu brain thermal tunnels (ABTTs) and to interface with the temperature therapy systems of the present disclosure in accordance with an exemplary embodiment of the present disclosure.

FIG. 49 shows a front perspective view of the glasses of FIG. 48.

FIG. 50 shows a side elevation view of the glasses of FIG. 48.

FIG. 51 shows a top plan view of the glasses of FIG. 48.

FIG. 52 shows a back perspective view of the glasses of FIG. 48.

FIG. 53 shows a perspective view of a pair of glasses with a temperature inductor positioned thereon to provide heat to a pair of Abreu brain thermal tunnels (ABTTs) and to interface with the temperature therapy systems of the present disclosure in accordance with another exemplary embodiment of the present disclosure.

FIG. 54 shows a back perspective view of the glasses of FIG. 53.

FIG. 55 shows a front elevation view of the glasses of FIG. 53.

FIG. 56 shows a back elevation view of the glasses of FIG. 53.

FIG. 57 shows a top plan view of the glasses of FIG. 53.

FIG. 58 shows a bottom plan view of the glasses of FIG. 53.

FIG. 59 shows a side elevation view of the glasses of FIG. 53.

FIG. 60 shows a perspective view of a pair of glasses with a temperature inductor positioned thereon to provide heat to a pair of Abreu brain thermal tunnels (ABTTs) and to interface with the temperature therapy systems of the present disclosure in accordance with still another exemplary embodiment of the present disclosure.

FIG. 61 shows a back perspective view of the glasses of FIG. 60.

FIG. 62 shows a front elevation view of the glasses of FIG. 60.

FIG. 63 shows a back elevation view of the glasses of FIG. 60.

FIG. 64 shows a top plan view of the glasses of FIG. 60.

FIG. 65 shows a bottom plan view of the glasses of FIG. 60.

FIG. 66 shows a side elevation view of the glasses of FIG. 60.

FIG. 67 shows a perspective view of a thermometer in accordance with an exemplary embodiment of the present disclosure.

FIG. 68 shows a front elevation view of the thermometer of FIG. 67.

FIG. 69 shows a side elevation view of the thermometer of FIG. 67.

FIG. 70 shows a top plan view of the thermometer of FIG. 67.

FIG. 71 shows a bottom plan view of the thermometer of FIG. 67.

FIG. 72 shows a perspective of a thermometer in accordance with another exemplary embodiment of the present disclosure.

FIG. 73 shows a front elevation view of the thermometer of FIG. 72.

FIG. 74 shows a top plan elevation view of the thermometer of FIG. 72.

FIG. 75 shows a side elevation view of the thermometer of FIG. 72.

FIG. 76 shows a bottom plan view of the thermometer of FIG. 72.

FIG. 77 shows a back elevation view of the thermometer of FIG. 72.

FIG. 78 shows a perspective view of a tongue heater or inductor in accordance with an exemplary embodiment of the present disclosure.

FIG. 79 shows a front elevation view of the tongue heater of FIG. 78.

FIG. 80 shows a top plan view of the tongue heater of FIG. 78.

FIG. 81 shows a side elevation view of the tongue heater of FIG. 78.

FIG. 82 shows a back elevation view of the tongue heater of FIG. 78.

FIG. 83 shows an exploded perspective view of the tongue heater of FIG. 78.

FIG. 84 shows a perspective view of a tongue heater or inductor in accordance with another exemplary embodiment of the present disclosure.

FIG. 85 shows a front elevation view of the tongue heater of FIG. 84.

FIG. 86 shows a top plan view of the tongue heater of FIG. 84.

FIG. 87 shows a back elevation view of the tongue heater of FIG. 84.

FIG. 88 shows a side elevation view of the tongue heater of FIG. 84.

FIG. 89 shows a perspective view of a tongue heater or inductor in accordance with still another exemplary embodiment of the present disclosure.

FIG. 90 shows a front elevation view of the tongue heater of FIG. 89.

FIG. 91 shows a top plan view of the tongue heater of FIG. 89.

FIG. 92 shows a back elevation view of the tongue heater of FIG. 89.

FIG. 93 shows a side elevation view of the tongue heater of FIG. 89.

FIG. 94 shows a perspective view of a tongue heater or inductor in accordance with yet another exemplary embodiment of the present disclosure.

FIG. 95 shows a front elevation view of the tongue heater of FIG. 94.

FIG. 96 shows a top plan view of the tongue heater of FIG. 94.

FIG. 97 shows a back elevation view of the tongue heater of FIG. 94.

FIG. 98 shows a side elevation view of the tongue heater of FIG. 94.

FIG. 99 shows an exploded perspective view of the tongue heater of FIG. 94.

FIG. 100 shows a head-supported nasal heater or inductor in accordance with an exemplary embodiment of the present disclosure.

FIG. 101 shows a perspective view of a head-mounted ABTT thermal heater or inductor in accordance with an exemplary embodiment of the present disclosure.

FIG. 102 shows a perspective view of a tongue heater or inductor in accordance with a further exemplary embodiment of the present disclosure.

FIG. 103 shows a perspective view of an aural or ear heater or inductor in accordance with an exemplary embodiment of the present disclosure.

FIG. 104 shows a facial mask with a separate heater providing airflow to the facial mask in accordance with an exemplary embodiment of the present disclosure.

FIG. 105 shows a perspective view of a chamber of a temperature therapy system in accordance with any of the embodiments disclosed herein and a heater reservoir attached to the chamber with a modified rear end wall in accordance with an exemplary embodiment of the present disclosure.

FIG. 106 shows a further view of the chamber and heater reservoir of FIG. 105.

FIG. 107 shows a side elevation view of a chamber for a temperature therapy system of the present disclosure with a patient support in a lowered position.

FIG. 108 shows the side elevation view of the chamber of FIG. 107 with the patient support in a raised position.

FIG. 109 shows the side elevation view of the chamber of FIG. 107 with the patient support lowered and retracted or moved out from under the chamber.

FIG. 110 shows the side elevation view of the chamber of FIG. 107 with the patient support retracted out from under the chamber and then raised.

FIG. 111 shows a top plan view of a chamber with an upper surface removed to show an internal configuration of heaters in the chamber in accordance with an exemplary embodiment of the present disclosure.

FIG. 112 shows a top plan view of a chamber with an upper surface removed to show an internal configuration of heaters in the chamber in accordance with another exemplary embodiment of the present disclosure.

FIG. 113 shows another top plan view of the chamber of FIG. 112.

FIG. 114 shows a top plan view of a chamber with an upper surface removed to show an internal configuration of heaters in the chamber in accordance with still another exemplary embodiment of the present disclosure.

FIG. 115 shows a top plan view of a chamber with an upper surface removed to show an internal configuration of heaters in the chamber in accordance with yet another exemplary embodiment of the present disclosure.

FIG. 116 shows a top plan view of a chamber with an upper surface removed to show an internal configuration of heaters in the chamber in accordance with an even further exemplary embodiment of the present disclosure.

FIG. 117 shows a top plan view of a chamber with an upper surface removed to show an internal configuration of heaters in the chamber in accordance with still yet another exemplary embodiment of the present disclosure.

FIG. 118 shows a top plan view of a chamber with an upper surface removed to show an internal configuration of heaters in the chamber in accordance with a yet even further exemplary embodiment of the present disclosure.

FIG. 119 shows a top plan view of a chamber with an upper surface removed to show an internal configuration of heaters in the chamber in accordance with an exemplary embodiment of the present disclosure.

FIG. 120 shows a perspective view of a front wall of a chamber in accordance with an exemplary embodiment of the present disclosure.

FIG. 121 shows a perspective view of a chamber in accordance with an exemplary embodiment of the present disclosure.

FIG. 122A shows a modification of heat shock protein levels with time during a sustained application of a specific frequency of temperature cycles.

FIG. 122B shows a temperature cycle in accordance with an exemplary embodiment of the present disclosure.

FIG. 123 shows a temperature cycle in accordance with another exemplary embodiment of the present disclosure.

FIG. 124 shows a temperature cycle in accordance with a further exemplary embodiment of the present disclosure.

FIG. 125 shows a temperature cycle in accordance with a still further exemplary embodiment of the present disclosure.

FIG. 126 shows a temperature therapy system in accordance with an exemplary embodiment of the present disclosure.

FIG. 127 shows a schematic view of alternating fluid temperatures flowing in an inductor circuit of the temperature therapy system of FIG. 126 in accordance with an exemplary embodiment of the present disclosure.

FIG. 128 shows a view of temperatures at a terminus of an ABTT from the fluid temperatures shown in FIG. 127.

FIG. 129 shows a schematic view of alternating fluid temperatures flowing in an inductor circuit of the temperature therapy system of FIG. 126 in accordance with another exemplary embodiment of the present disclosure.

FIG. 130 shows a view of temperatures at a terminus of an ABTT from the fluid temperatures shown in FIG. 129.

FIG. 131 shows a temperature therapy system in accordance with another exemplary embodiment of the present disclosure.

FIG. 132 shows a side view of a thermal therapy system in accordance with an exemplary embodiment of the present disclosure.

FIG. 133 shows a perspective view of a portion of the thermal therapy system of FIG. 132.

FIG. 134 shows an enlarged view of a portion of the thermal therapy system of FIG. 132.

FIG. 135 shows a perspective view of another thermal therapy system in accordance with an exemplary embodiment of the present disclosure.

FIG. 136 shows an enlarged view of a portion of the thermal therapy system of FIG. 135.

FIG. 137 shows a view of a further thermal therapy system in accordance with an exemplary embodiment of the present disclosure.

FIG. 138 shows a view of a still further thermal therapy system in accordance with an exemplary embodiment of the present disclosure.

FIG. 139 shows an even further thermal therapy system in accordance with an exemplary embodiment of the present disclosure.

FIG. 140 shows a side view of a thermoelectric device of the thermal therapy system of FIG. 139.

FIG. 141 shows yet another thermal therapy system in accordance with an exemplary embodiment of the present disclosure.

FIG. 142A shows a thermal device in accordance with an exemplary embodiment of the present disclosure.

FIG. 142B shows a further thermal device in accordance with an exemplary embodiment of the present disclosure.

FIG. 143A shows another thermal device in accordance with an exemplary embodiment of the present disclosure.

FIG. 143B shows still another thermal device in accordance with an exemplary embodiment of the present disclosure.

FIG. 144 shows yet another thermal device in accordance with an exemplary embodiment of the present disclosure.

FIG. 145 shows a headband configured to support a thermal device in accordance with an exemplary embodiment of the present disclosure.

FIG. 146 shows a further headband configured to support a thermal device in accordance with an exemplary embodiment of the present disclosure.

FIG. 147 shows a still further headband configured to support a thermal device in accordance with an exemplary embodiment of the present disclosure.

FIG. 148 shows a process of treating diseases and conditions in accordance with an exemplary embodiment of the present disclosure.

FIG. 149 shows a head-mounted thermal device in accordance with an exemplary embodiment of the present disclosure positioned on a head of a patient.

FIG. 150 shows another view of the head-mounted thermal device of FIG. 149.

FIG. 151 shows another head-mounted thermal device in accordance with an exemplary embodiment of the present disclosure.

FIG. 152 shows a front view of the head-mounted thermal device of FIG. 151.

FIG. 153 shows a side view of the head-mounted thermal device of FIG. 151.

FIG. 154 shows a bottom view of the head-mounted thermal device of FIG. 151.

FIG. 155 shows a partially exploded perspective view of the head-mounted thermal device of FIG. 151.

FIG. 156 shows a view of a thermal therapy system in accordance with an exemplary embodiment of the present disclosure.

FIG. 157 shows a perspective view of a head-mounted thermal device in accordance with an exemplary embodiment of the present disclosure.

FIG. 158 shows another perspective view of the head-mounted thermal device of FIG. 157.

FIG. 159 shows a side view of another head-mounted thermal device in accordance with an exemplary embodiment of the present disclosure.

FIG. 160 shows a rear view of the head-mounted thermal device of FIG. 159.

FIG. 161 shows a top view of the head-mounted thermal device of FIG. 159.

FIG. 162 shows a partially exploded perspective view of the head-mounted thermal device of FIG. 159.

FIG. 163 shows a perspective view of a portion of a patient support in accordance with an exemplary embodiment of the present disclosure.

### DETAILED DESCRIPTION

While traditional medicine in the form of surgery and medication has been successful in treating an array of human diseases and conditions, the inventor of the inventions of the present disclosure has come to realize that traditional approaches to the treatment of diseases and conditions have limits. Indeed, there are many diseases and conditions for which our current medical knowledge is insufficient to provide effective, reliable treatments.

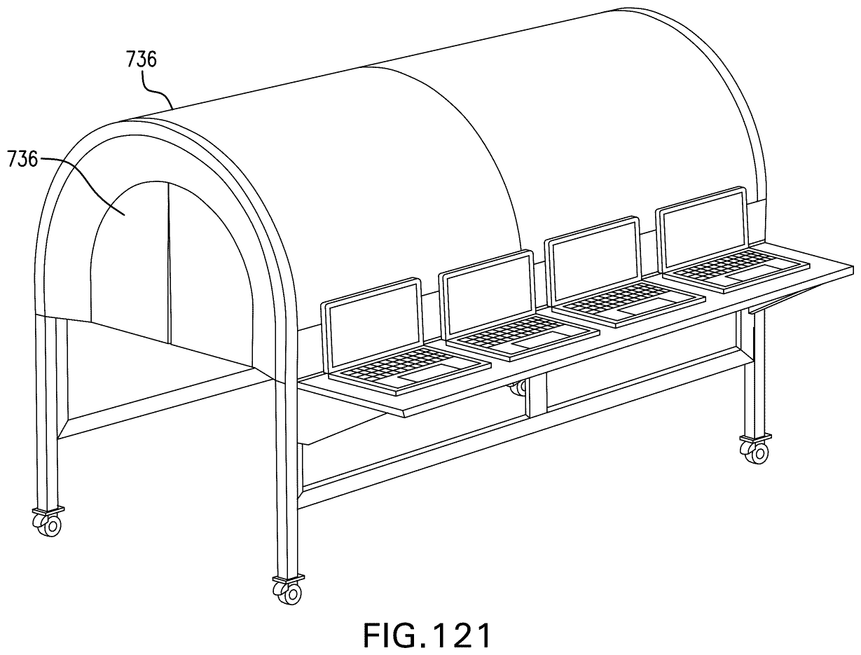

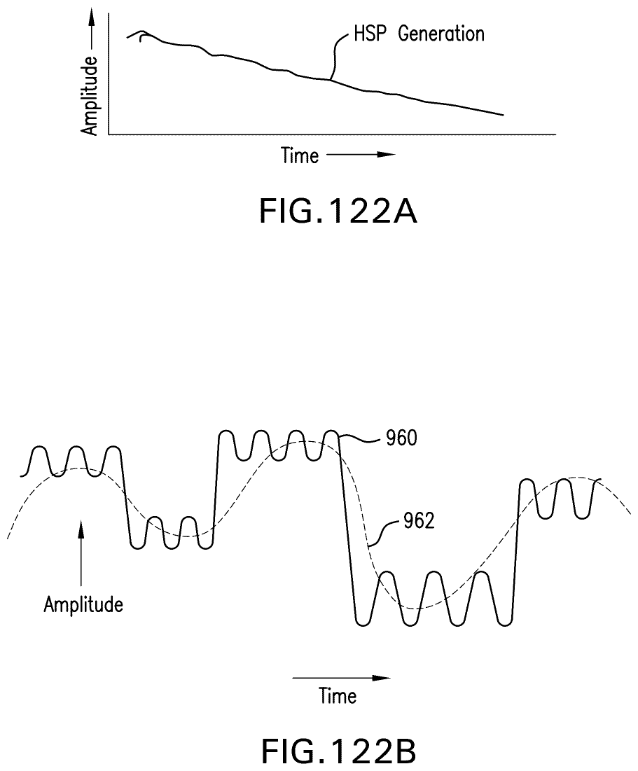

In view of the medical present environment, the inventor determined through extensive experimentation that humans have temperature cycles. In the context of this disclosure, a temperature cycle is an alternation between an upper temperature and a lower temperature relative to a baseline. As an example, a baseline temperature can be, for example, 98 degrees fahrenheit. Temperature applied to a patient having a measured or identified temperature cycle can then be elevated, for example, intermittently for short periods, to approximately 122 degrees fahrenheit, which is approximately 50 degrees Celsius of environmental temperature. On a low or reduced temperature side, the temperature can intermittently be as low as approximately 1 degree Celsius, which is approximately 33.8 degrees fahrenheit of environmental temperature. Additionally, because the various devices disclosed herein are either continuously in contact with ABTT 90, or the temperatures are applied continuously in transition from one temperature to another, the cycles described herein are “continuous” cycles. In other words, there are no step functions such that temperature at one level or value is discontinuous with a temperature at another level or value. See FIGS. 122-125 for examples of temperature cycles.

The inventor further determined that applying temperature cycles to a patient causes modulation, neutralization, or reduction of harmful molecules including inflammatory markers and cytokines as well as generation or increase of protective molecules and remodeling of molecules, including proteins. Such protective molecules can include heat shock proteins, also known by their initials as HSP, particularly heat shock proteins that are beneficial in combatting specific diseases and conditions. More specifically, the applied heat cycles from the temperature therapy systems disclosed herein help to generate heat shock proteins and reduce or normalize inflammatory molecules such as cytokines, for example, tumor necrosis factor alpha (TNF alpha biomarker), or interleukins, including but not limited to interleukin 2-6-13, and interleukin 17, and cancer-signaling molecules, including, but not limited to C-reactive proteins CA-19-9, CA-125, and CA 27-29. As one example, the system and method of the present disclosure are configured to specifically maintain, refold, or generate the P53 protein for protection against and treatment of cancer, while eliminating toxic wastes, which can be described as toxic aggregates caused by misfolded proteins, such as beta-amyloid in Alzheimer's disease, alpha-synuclein in Parkinson's disease, polyglutamine in ataxia, and TDP-43 in amyotrophic lateral sclerosis (ALS), and other diseases and conditions that associated with misfolded proteins such as amylin in diabetes. HSP70 is also generated, which is a broadly protective HSP. As another example, the systems disclosed herein can normalize hormone levels, as well as the normalization of neurotransmitters. Additional examples and embodiments are disclosed in detail herein.

It should be apparent from the description above that the temperature therapy systems and methods disclosed herein act at a molecular level in the human body. Indeed, the systems and methods disclosed herein act to redefine a patient's body's internal health landscape, working with the brain to leverage the body's natural biology by provoking and encouraging the production of beneficial chemicals and molecules while reducing or normalizing harmful molecules, chemistry, and substances.

Further, the systems and method of the present disclosure, which can be broadly described as brain-guided thermal therapies, both hyperthermia and hypothermia, in particular brain-guided hyperthermia (BgH), have been shown through testing to be effective in the treatment of twenty-eight distinct illnesses or conditions that have traditionally be deemed to be intractable, illustrating the extraordinary potential of BgH therapy in restoring lost brain function and systemic function for particularly challenging health conditions. More specifically, those illnesses and conditions are, as follows: Alzheimer's disease, amyotrophic lateral sclerosis (ALS), anoxic encephalopathy, cancer, corticobasal degeneration, dementia, Devic's disease, epilepsy, flail arm, Friedreich ataxia, herpes, Huntington's disease, immune system related issues, inclusion-body myositis, lupus, Lyme disease, Machado-Joseph disease, multiple sclerosis (MS), multiple system atrophy, muscular dystrophy, Parkinson's disease, primary lateral sclerosis, progressive supranuclear palsy, spastic paralysis, spinal cord injury, spinal muscular atrophy, spinocerebellar ataxia (SCA2, SCA6), transverse myelitis, traumatic brain injury.

The inventor determined that the benefits provided herein are brain-activated. More specifically, such activation is controlled by the hypothalamus. Accordingly, there are two key aspects of the operation of the present disclosure. First, the application of heat and reduction, removal, or decrease of heat at specific amplitudes and frequencies at the ABTT 90. Second, the application of heat and reduction, removal, or decrease of heat at specific amplitudes and frequencies to the whole body of a patient 82. The inventor also determined that whole-body thermal treatments can be applied to the entire body, including the head of patient 82. Alternatively, heat to ABTT 90 can be at different amplitudes and frequencies as compared to heat applied to the body of patient 82. Regardless of how and where such heat is applied, the result is that the brain is prompted to behave chemically in a specific way.

In the context of this disclosure, the reduction or removal of heat is defined as being from a previously applied level, typically measured by temperature. Conversely, the increase, application, or transference of heat is based on a temperature level higher than a currently applied temperature or heat level and/or higher than a baseline temperature of patient 82. In an example, if a temperature of 50 degrees Celsius is applied to patient 82 at a predetermined amplitude and frequency, and the temperature is reduced to a lower value, than 50 degrees Celsius, then heat is being removed or reduced based on the previous value of 50 degrees Celsius. As another example, if a patient has a nominal internal temperature of 37 degrees Celsius and a temperature of 30 degrees Celsius is applied to patient 82, at ABTT 90 or to the body of patient 82, then heat is being removed or decreased.

Also, in the context of this disclosure, all temperatures and frequencies have been predetermined based on extensive analysis of temperatures and frequencies applied to test subjects in controlled environments.

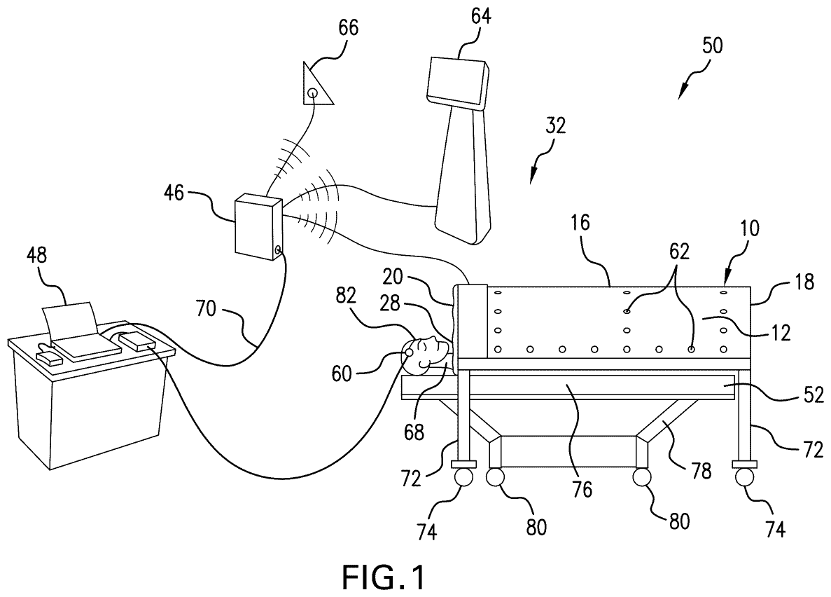

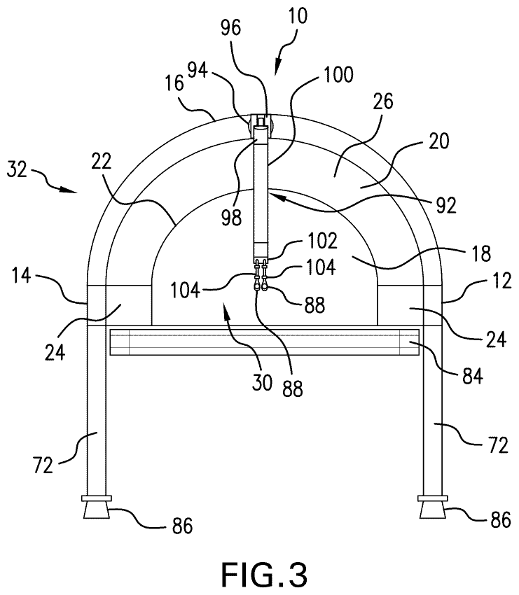

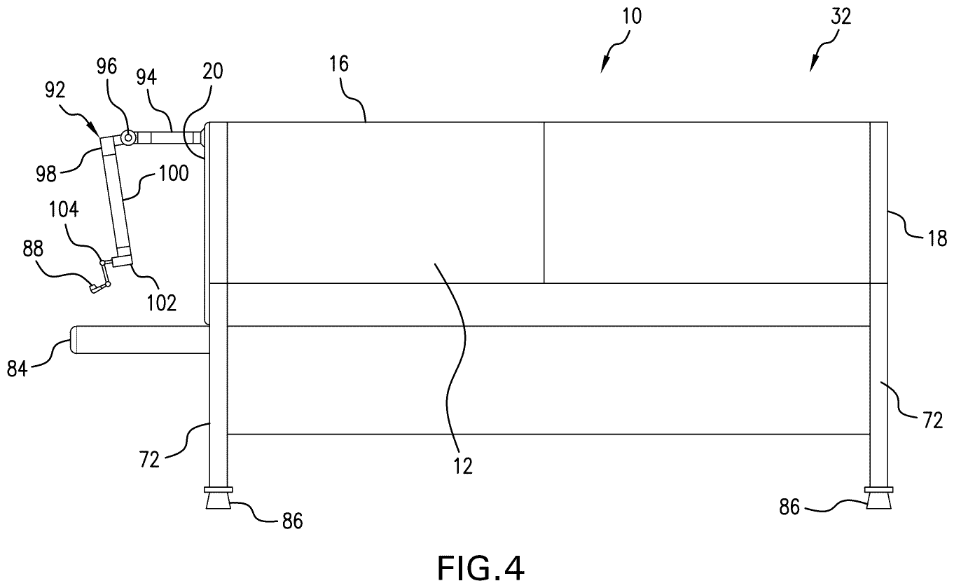

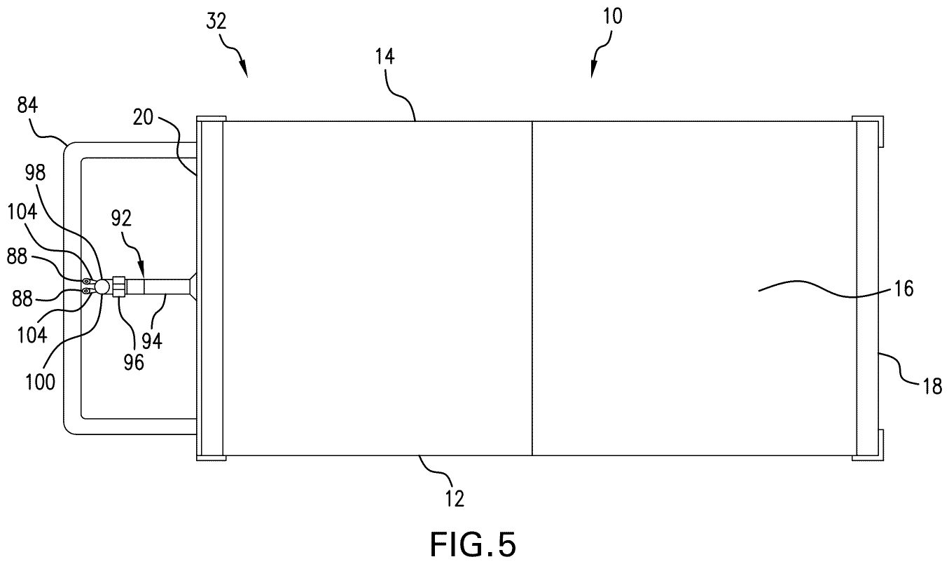

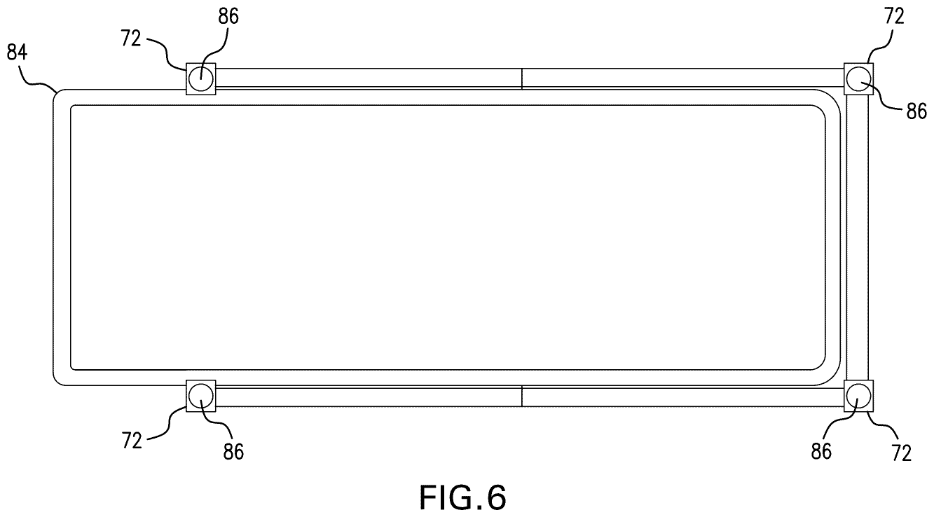

FIGS. 1-8 shows views of a temperature therapy or therapy system 50. As discussed in more detail herein, temperature therapy system 50 includes a chamber 10, a plurality of heaters 36, a power supply 46, a computer 48 including a processor and a non-transitory computer-readable medium, and a patient support 52. The processor is physically connected to and communicates with the non-transitory computer-readable medium, which can store software the processor can execute as part of therapy system 50 to operate the hardware of temperature therapy system 50. Throughout this disclosure, since computer 48 includes one or more processors, and since the functions of computer 48 are conducted, carried out, or executed by the processor, reference number 48 can also be used to refer to the processor. On the other hand, the non-transitory computer-readable medium is typically a separate element that can be internal to computer 48 or can be external to computer 48. Accordingly, reference number 48 is not used to refer to the non-transitory computer-readable medium.

In addition, many aspects of the disclosure are described in terms of sequences of actions to be performed by elements of a computer system or other hardware capable of executing programmed instructions, for example, a general-purpose computer, special-purpose computer, workstation, or other programmable data process apparatus. It will be recognized that in each of the embodiments, the various actions could be performed by specialized circuits (e.g., discrete logic gates interconnected to perform a specialized function), by program instructions (software), such as program modules, being executed by one or more processors (e.g., one or more microprocessors, a central processing unit (CPU), and/or application specific integrated circuit), or by a combination of both. For example, embodiments can be implemented in hardware, software, firmware, microcode, or any combination thereof. The instructions can be program code or code segments that perform necessary tasks and can be stored in a non-transitory machine-readable medium such as a storage medium or other storage(s). A code segment may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents.

The non-transitory machine-readable medium can additionally be considered to be embodied within any tangible form of computer-readable carrier, such as solid-state memory, magnetic disk, and/or optical disk containing an appropriate set of computer instructions, such as program modules, and data structures that would cause a processor to carry out the techniques described herein. A computer-readable medium may include the following: an electrical connection having one or more wires, magnetic disk storage, magnetic cassettes, magnetic tape or other magnetic storage devices, a portable computer diskette, a random access memory (RAM), a read-only memory (ROM), an erasable programmable read-only memory (e.g., EPROM, EEPROM, or Flash memory), or any other tangible medium capable of storing information. It should be noted that the system of the present disclosure is illustrated and discussed herein as having various modules and units that perform particular functions.

It should be understood that these modules and units are merely described based on their function for clarity purposes, and do not necessarily represent specific hardware or software. In this regard, these modules, units, and other components may be hardware and/or software implemented to substantially perform their particular functions explained herein. The various functions of the different components can be combined or segregated as hardware and/or software modules in any manner and can be useful separately or in combination. Input/output or I/O devices or user interfaces including, but not limited to, keyboards, displays, pointing devices, and the like can be coupled to the system either directly or through intervening I/O controllers. Thus, the various aspects of the disclosure may be embodied in many different forms, and all such forms are contemplated to be within the scope of the disclosure.

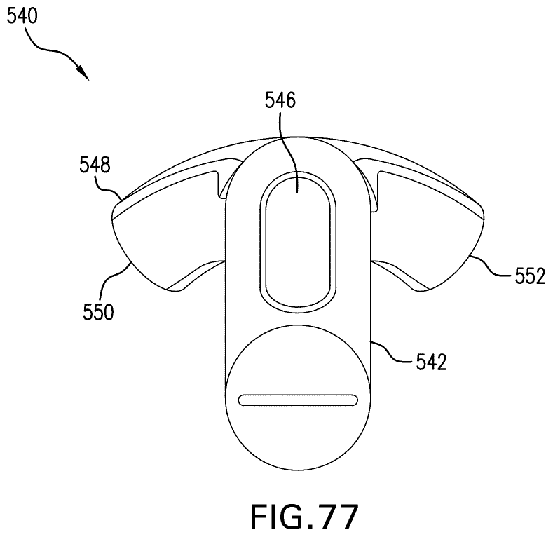

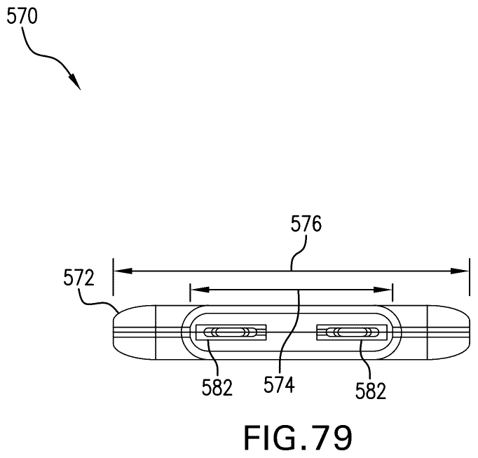

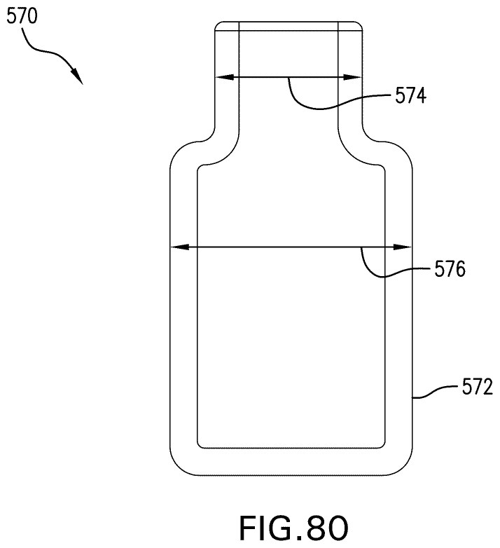

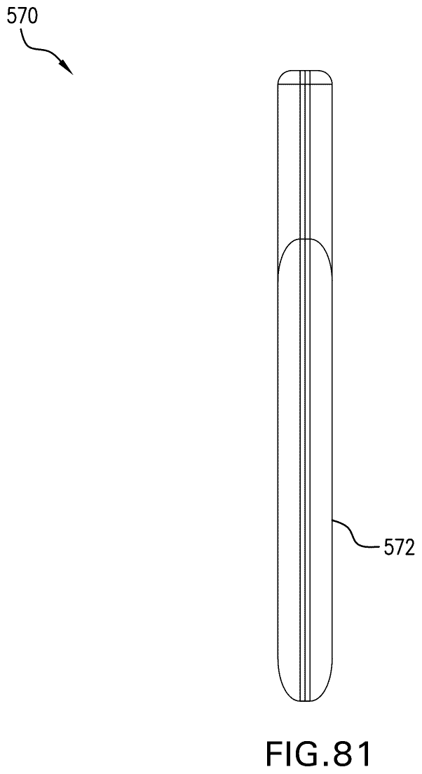

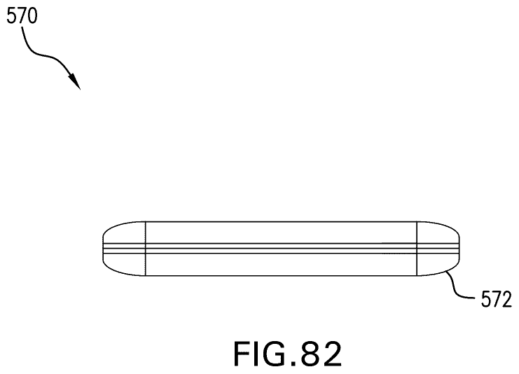

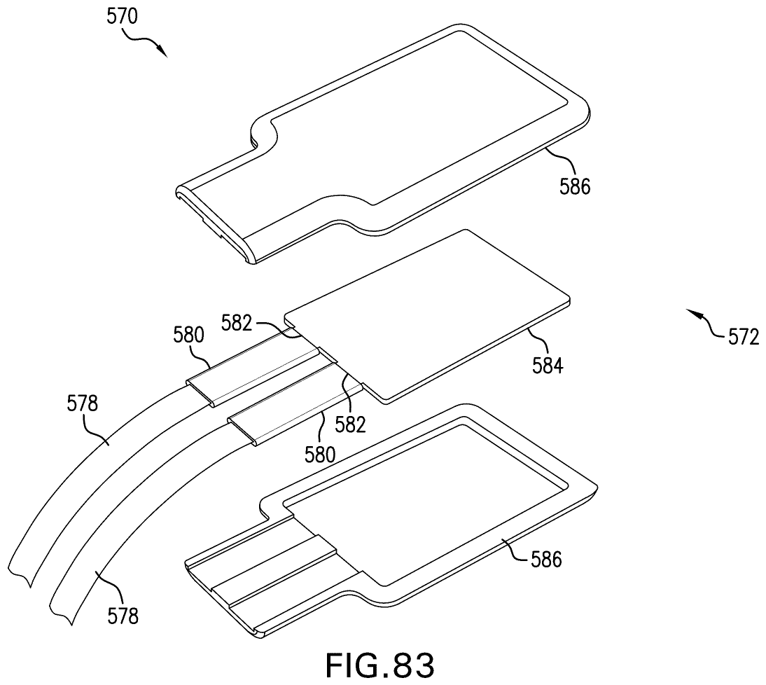

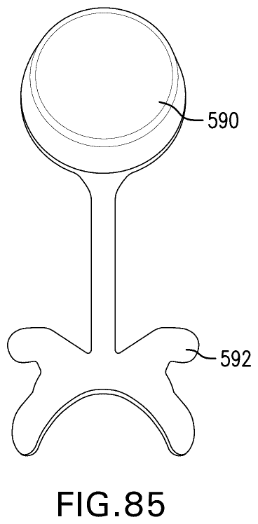

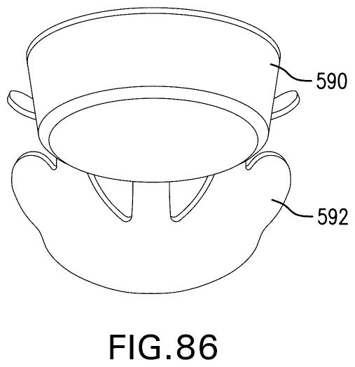

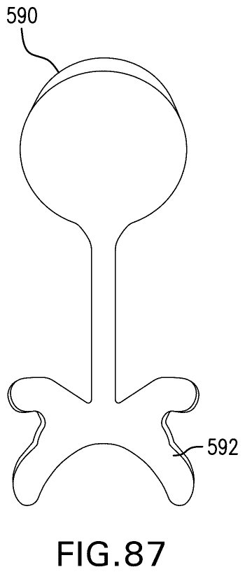

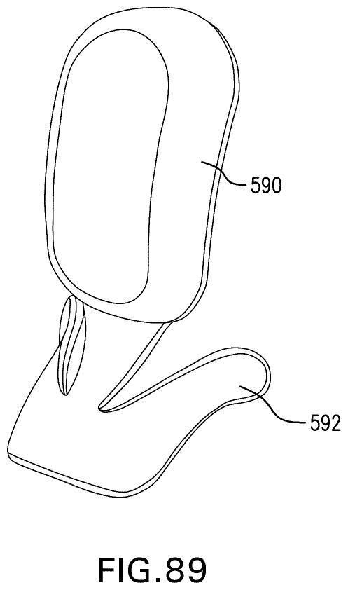

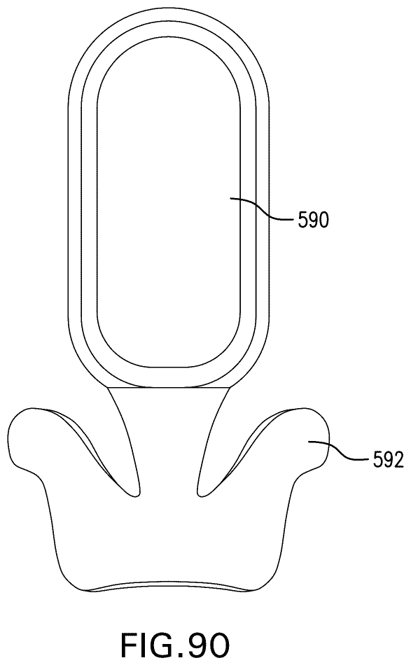

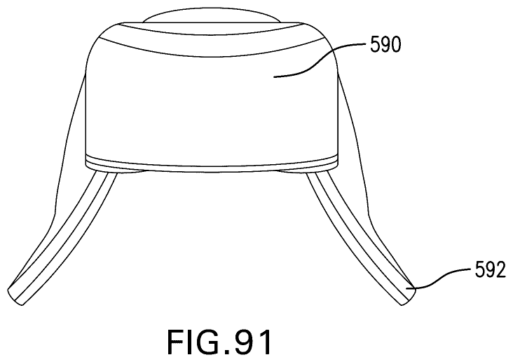

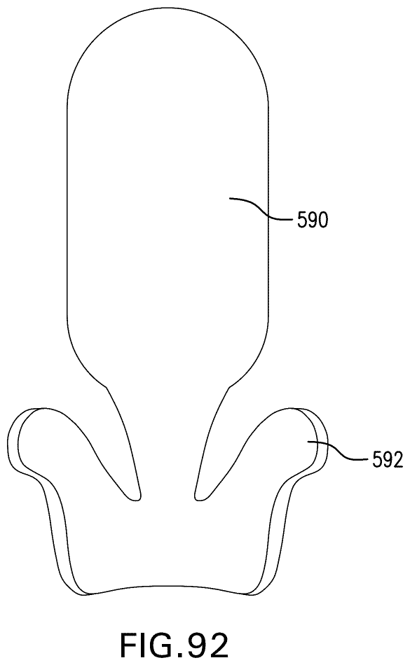

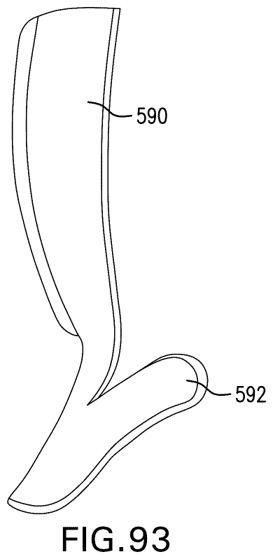

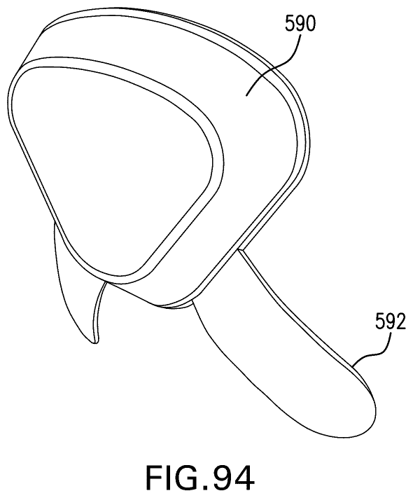

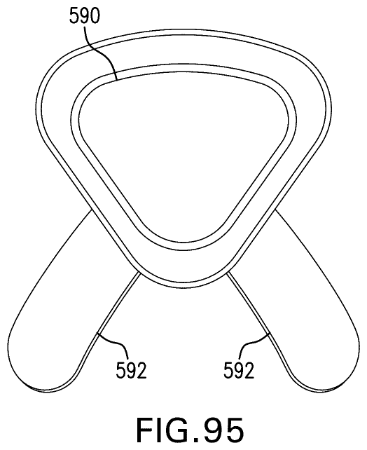

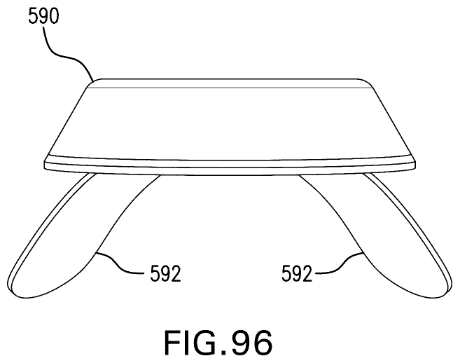

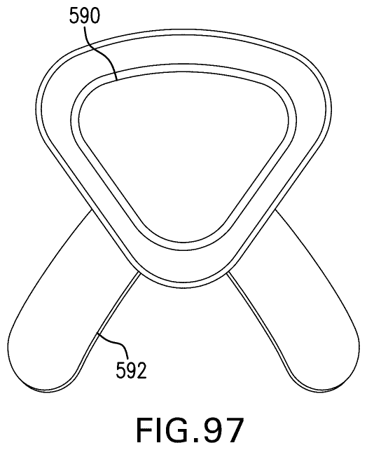

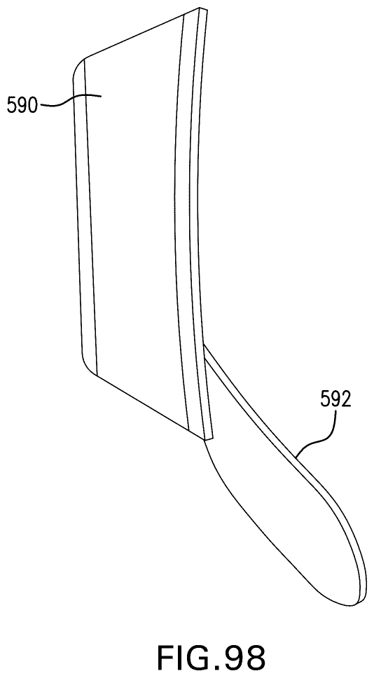

Temperature therapy system 50 can also include a plurality of inductors, including an oral inductor 54, a tympanic inductor 56, a lung inductor 58, one or more patient temperature sensors 60, and a plurality of chamber temperature sensors 62. See also FIGS. 77-104 for additional inductor embodiments. Temperature therapy system 50 can also include a separate computer 64 that includes a processor and a non-transitory computer-readable medium. System 50 can include an antenna 66 that enables remote communication with system 50. Accordingly, a medical professional can remotely control the functions of system 50 by way of a cellular network, IEEE 802.11, Wi-Fi, BLE (Bluetooth 4.0, Bluetooth low energy), Bluetooth, IEEE 802.15.4, Zigbee, Z-Wave, 6LoWPAN, NB-IOT, LoRaWAN, or other radio frequency standards or protocols. While less preferred due to the widespread availability of various radiofrequency systems such as cellular, Bluetooth, and Wi-Fi, optical controllers can also be used even though they typically require a line of sight.

Chamber 10 includes a first sidewall 12 and a second sidewall 14 that extend approximately parallel to each other along a longitudinal length of chamber 10. Approximately parallel in the context of this disclosure is parallel within the capability of conventional manufacturing processes. As long as all other conditions in this disclosure are met, the parallelism of second sidewall 14 with first sidewall 12 can be broad. For example, a preferred parallelism is less than or equal to 10 degrees. A more preferred parallelism is less than or equal to 5 degrees. An even more preferred parallelism is less than or equal to 1 degree. A most preferred parallelism is less than or equal to 0.5 degrees.

Chamber 10 further includes a top or upper wall 16. Top or upper wall 16 extends from an upper location on first sidewall 12 to an upper location on second sidewall 14. Top or upper wall 16 can extend directly from first sidewall 12 to second sidewall 14 along a shortest distance from first sidewall 12 to second sidewall 14, or top or upper wall 16 can be in the shape of a convex arc that is in a shape of a circulate or oval arc shape extending from first sidewall 12 to second sidewall 14.

Chamber 10 also includes a rear end wall 18, which can be a solid wall, or can include an opening for a heater and/or cooler and/or combination heater/cooler. In an embodiment, rear end wall 18 is directly connected to first sidewall 12, second sidewall 14, and top or upper wall 16, and thus is integrally formed and contiguous with first sidewall 12, second sidewall 14, and top or upper wall 16. In an alternative embodiment, rear end wall 18 can be indirectly connected to, for example, first sidewall 12 by a hinge, such as a piano or other type hinge, and rear end wall 18 can be removably connectable to second sidewall 14 and top or upper wall 16 by way of latches, clamps, fasteners, and the like. In a further embodiment, rear end wall 18 can be directly or indirectly removably attached to one or more of first sidewall 12, second sidewall 14, and top or upper wall 16. Rear end wall 18 is considered an end wall because it is near where the feet of a patient would be when the patient is positioned within chamber 10.

Chamber 10 further includes a front wall 20, which can also be described as front end or front wall 20 since front wall 20 is at an opposite end of chamber 10 from rear end wall 18. Additionally, front wall 20 is near to where a head 83 of patient 82 is located when patient 82 is positioned within interior 30 and can also be a location where patient 82 is moved into interior 30. In an embodiment, front wall 20 is directly connected to first sidewall 12, second sidewall 14, and top or upper wall 16, and thus is integrally formed and contiguous with first sidewall 12, second sidewall 14, and top or upper wall 16. In an alternative embodiment, front wall 20 can be indirectly connected to, for example, first sidewall 12 by a hinge, such as a piano or other type hinge, and front wall 20 can be removably connectable to second sidewall 14 and top or upper wall 16. In a further embodiment, front wall 20 can be directly or indirectly removably attached to one or more of first sidewall 12, second sidewall 14, and top or upper wall 16.

Front wall 20 includes an opening 22 that is sized and dimensioned to conform approximately to the size of a neck 68 of a human. Opening 22 can be called a front opening since opening 22 is formed in front wall 20. Opening 22 can be formed as two vertically extending sides 24 and an arced side 26 that extends upwardly from vertically extending sides 24, or as a continuous arc in a form similar to arced side 26. Approximately in the context of opening 22 means a size that directly contacts neck 68 of the patient, to a size that can include a gap with the patient's neck of several inches. To minimize heat loss from chamber 10, the size of any gap between neck 68 of patient 82 and sides 24 and arced side 26 is preferably as small as possible, including line-to-line or touching contact between sides 24 and arced side 26 and neck 68 of patient 82.

Front wall 20 as disclosed above is a relatively rigid wall. A portion of front wall 20 can be in a form of a flexible interface or adapter 21. Adapter 21 can include a conformable bottom edge 28. An advantage of conformable bottom edge 28 is to provide close positioning of conformable bottom edge 28 to the neck of the patient. Indeed, conformable bottom edge 28 permits enabling contact of conformable bottom edge 28 with the neck of the patient. The material of adapter 21 can be, for example, a heat-resistant cloth fabricated from a material such as neoprene, silicone, ceramic, refractory, and other materials suitable to be exposed to temperatures up to 100 degrees Celsius or 212 degrees fahrenheit, which can occur near the top of chamber 10. While conformable bottom edge 28 is described as being conformable, it should be understood that the entirety of adapter 21 is formed of a conformable material, and those portions of adapter 21 adjacent to neck 68 conform to the shape of neck 68.

The walls of chamber 10 define an interior 30 of chamber 10 that is configured to enclose patient 82 at least on an upper side of patient 82, or may entirely enclose patient 82, including, in an embodiment, head 83 and neck 68. In another embodiment, a portion of neck 68 and head 83 can extend outside chamber 10. More specifically, interior 30 is defined by at least the presence of first sidewall 12, second sidewall 14, top or upper wall 16, rear end wall 18, front wall 20, and adapter 21. Exterior 32 is all locations outside the volume that is defined or partially enclosed by first sidewall 12, second sidewall 14, top or upper wall 16, rear end wall 18, front wall 20, and adapter 21. Indeed, in a preferred embodiment a prone patient 82 is enclosed from the neck to the feet about a periphery of patient 82's body and over a top of patient 82's body by first sidewall 12, second sidewall 14, top or upper wall 16, rear end wall 18, front wall 20, and adapter 21. It should be noted that a bottom of chamber 10 may be generally or mostly open, typically near or below a lower side of patient 82's body. The bottom of chamber 10 can also be covered, for example, by a patient support, so that the body of patient 82 is entirely enclosed within chamber 10. In an embodiment, head 83 and neck 68 could also be enclosed in chamber 10. In another embodiment, head 83 and a portion of neck 68 can extend outside chamber 10. If neck 68 and head 83 are enclosed within chamber 10, the temperature of head 83 must be monitored to ensure the temperature of head 83 is maintained within safe limits. Reinforcements 34 may extend along a bottom edge of first sidewall 12, second sidewall 14, top or upper wall 16, and rear end wall 18. Reinforcements 34 increase the rigidity of the walls of chamber 10.

Temperature sensors 62 can be configured as shown in FIG. 1. It should be apparent that temperature sensors 62 are positioned to measure a plurality of temperatures within interior 30. In a configuration, temperature sensors 62 can extend along a length of first sidewall 12 and second sidewall 14 along a lower or bottom edge of first sidewall 12 and second sidewall 14. For example, temperature sensors 62 can be positioned within 150 mm of the bottom-most edge of first sidewall 12 and second sidewall 14. More preferably, temperature sensors 62 can be positioned within 100 mm of the bottom-most edge of first sidewall 12 and second sidewall 14. In an even more preferred embodiment, temperature sensors 62 can be positioned within 50 mm of the bottom-most edge of first sidewall 12 and second sidewall 14.

Temperature sensors 62 can extend along the lower or bottom edge of first sidewall 12 and second sidewall 14 from a location near to front wall 20 to a location near to rear end wall 18. Because of convection, conduction, and airflow, having many temperature sensors 62 is preferred to analyze the temperature applied to the whole body of patient 82 extending along the lower or bottom edge of first sidewall 12 and second sidewall 14. Accordingly, in an embodiment, there may be at least four temperature sensors 62 extending along the lower or bottom edge of first sidewall 12 and second sidewall 14. In a more preferred embodiment, there may be at least six temperature sensors 62 extending along the lower or bottom edge of first sidewall 12 and second sidewall 14. In an even more preferred embodiment, there may be seven temperature sensors 62 up to a temperature sensor 62 every inch (about 25 mm) of length of first sidewall 12 and second sidewall 14 for a total of 84 temperature sensors 62 along each of first sidewall 12 and second sidewall 14.

Temperature sensors 62 can also extend vertically from the lower bottom edge of first sidewall 12 and second sidewall 14 up to and along top or upper wall 16. Temperature sensors 62 can extend vertically in a plurality of locations. For example, temperature sensors 62 can extend vertically from the bottom edge of first sidewall 12 and second sidewall 14 in an area of first sidewall 12 and second sidewall 14 that is alongside or adjacent to front wall 20 up to top or upper wall 16. Additionally, temperature sensors 62 can extend vertically from the bottom edge of first sidewall 12 and second sidewall 14 in an area of first sidewall 12 and second sidewall 14 that is alongside rear end wall 18 up to top or upper wall 16. Temperature sensors 62 can extend vertically in other locations. For example, temperature sensors 62 can extend vertically from the bottom edge of first sidewall 12 and second sidewall 14 to top or upper wall 16 at a location that is between a horizontal midpoint of first sidewall 12 or second sidewall 14 and rear end wall 18.

The spacing of temperature sensors 62 in the vertical direction can be similar to the spacing of temperature sensors 62 extending along the bottom edges of first sidewall 12 and second sidewall 14. Because of convection, conduction, and airflow, having many temperature sensors 62 extending vertically is preferred to verify the actual temperature to which patient 82 is exposed, particularly when patient support 52 is moved vertically. Accordingly, the number of temperature sensors 62 extending in a vertical direction can be a minimum of three, including temperature sensor 62 positioned on the bottom edge of first sidewall 12 or second sidewall 14 to the temperature sensor positioned near or on top or upper wall 16. However, more temperature sensors 62 than three is preferred. Accordingly, the number of temperature sensors 62 can be 4-36, with spacing being from 500 mm to 25 mm, depending on the expected temperature gradient in the vertical direction. The spacing should always be smaller near the bottom half of first sidewall 12 and second sidewall 14 since this area is most likely where patient 82 will be located.

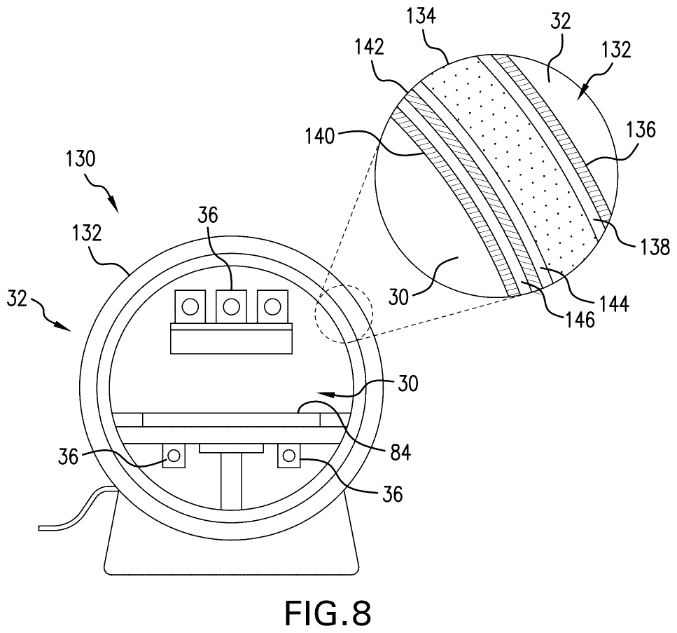

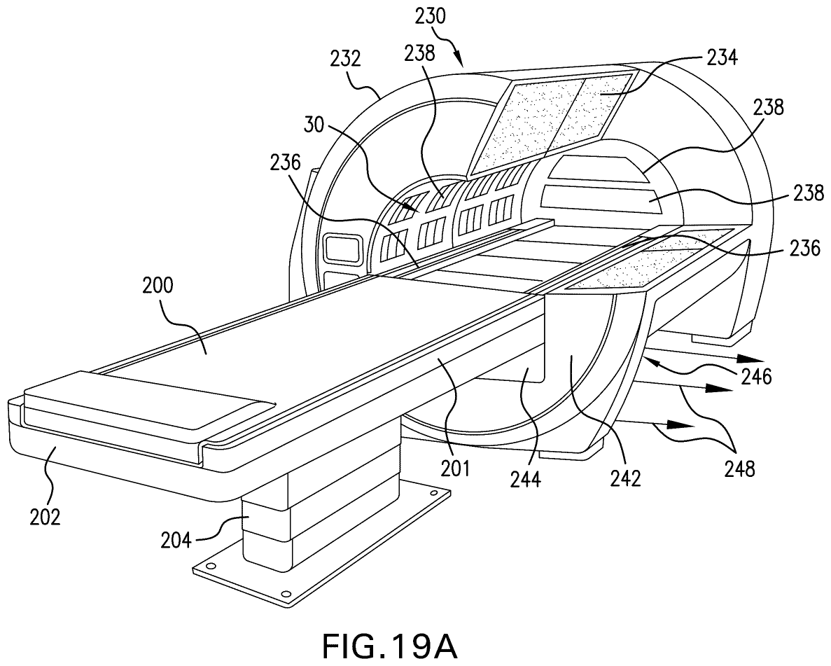

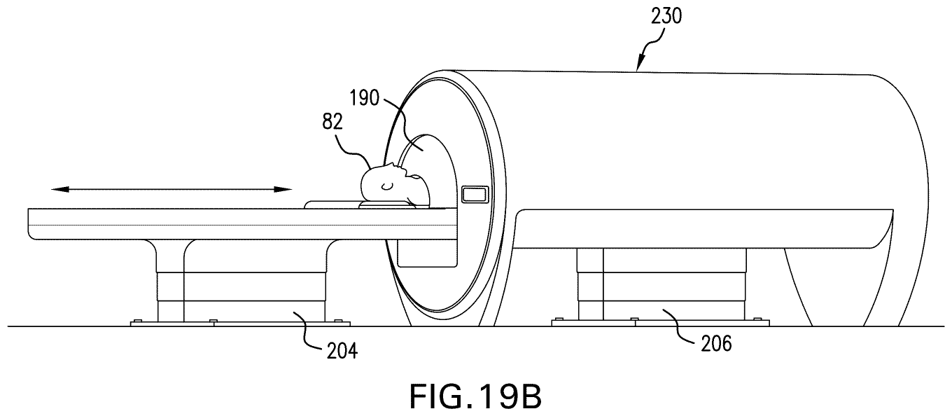

In FIG. 19A, and other figures, there can be a heater 201 in the bed or patient support 200 or below patient support 200. In FIG. 8, the plurality of heaters 36 are positioned on one or more interior surfaces of first sidewall 12, second sidewall 14, top or upper wall 16, and rear end wall 18. The plurality of heaters 36 can be in a plurality of configurations, including square and rectangular. More specifically, the plurality of heaters 36 can be positioned on interior surface 38 of first sidewall 12, interior surface 40 of second sidewall 14, interior surface 42 of top or upper wall 16, and interior surface 44 of rear end wall 18.

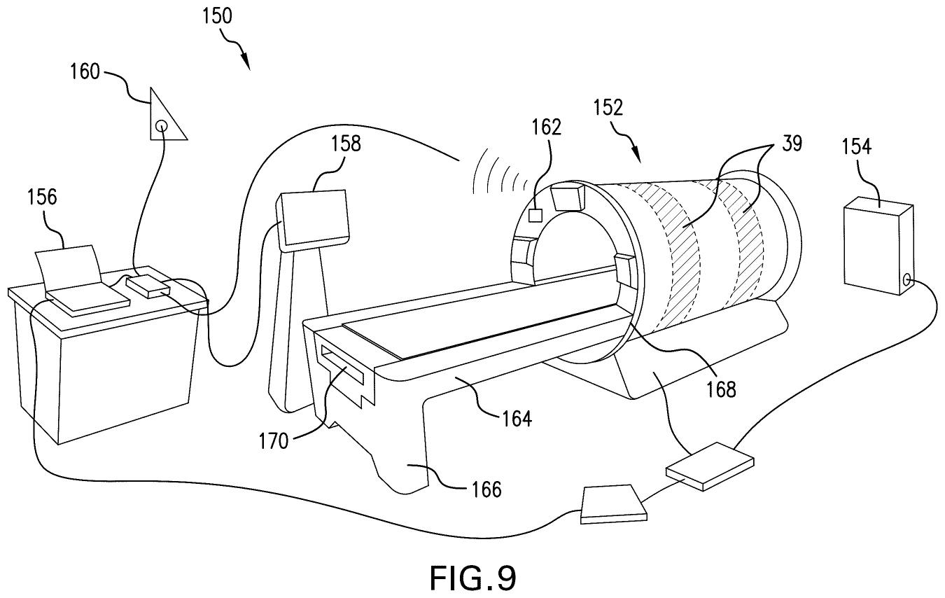

FIG. 9 shows another configuration of heaters 39. Heaters 39 have a width that extends a spaced distance along the longitudinal length of chamber 152. There can be a plurality of heaters 39. As shown in FIG. 9, two bands of heaters 39 are shown. Each band extends from near a bottom side of chamber 152, along the first side of chamber 152, across the top of chamber 152, and along the second, opposite side of chamber 152 from the first side to the bottom side of chamber 152 on the second side of chamber 152. Heaters 39 can each have a width that extends, for example, a width of 50 mm, a width of 100 mm, a width of 150 mm, a width of 200 mm, a width of 250 mm, a width of 300 mm, etc. While heaters 39 are shown as separate units, in an embodiment, heaters 39 can extend contiguously along a length of chamber 152. Heaters 39 and other heaters disclosed herein can be powered independently of each other for generating specific areas of more intense heat or less intense heat within chamber 10.

Power to the plurality of heaters 36 can be provided by power supply 46 positioned external to chamber 10. Power supply 46 can be controlled directly by an operator or can be controlled automatically by processor 48 positioned external to chamber 10. Processor 48 can communicate with power supply 46 wirelessly, such as by Bluetooth, Wi-Fi, or other wireless technologies as described elsewhere herein, or by a wire 70.

Chamber 10 can be supported by a plurality of legs 72. Each leg 72 may include a roller or wheels 74 to enable easy movement of chamber 10 along a floor. While not shown, one or more rollers or wheels 74 can include a brake to fix the position of an associated wheel 74.

Patient support 52 in the first embodiment shown in FIG. 1 includes a bed or mattress 76, a frame 78 that supports bed or mattress 76, and rollers or wheels 80 positioned at a lowest most end of frame 78 to enable movement of patient support 52 and a supported patient 82. As with rollers/wheels 74, one or more rollers or wheels 80 can include a brake to fix the position of associated roller/wheel 80. Frame 78 can be configured to raise and lower the level of bed or mattress 76. The ability to raise and lower can be valuable because chamber 10 provides a volume of air that is heated at a relatively high elevated level, described in more detail herein. The key to successfully treating patient 82 is quickly applying heat to and removing heat from patient 82.

One type of apparatus that can quickly apply and remove heated air is chamber 10 in combination with patient support 52. More specifically, because patient support 52 is configured to raise and lower bed/mattress 76 while supporting patient 82, patient support 52 can move patient 82 upwardly into the heated, enclosed, volume of chamber 10 to expose patient 82 to the elevated temperature level enclosed in chamber 10 for a predetermined time, period, or time interval, and then lower patient 82 to quickly remove patient 82 from the elevated temperature environment, thus controlling the temperature cycles to which patient 82 is exposed.

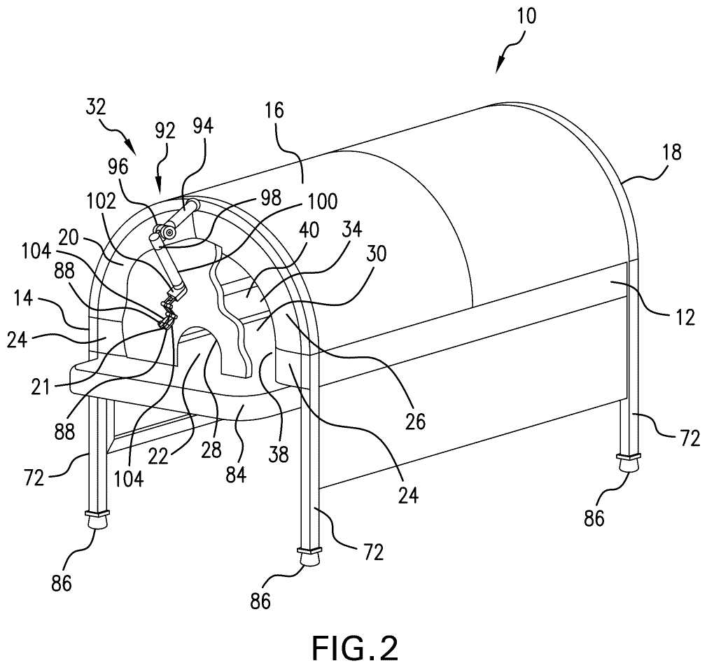

FIG. 2 shows a second embodiment bed or mattress 84. Bed or mattress 84 is fully supported by the structure of chamber 10. In addition, mattress 84 can be slidably supported by chamber 10 such that bed or mattress 84 can be quickly slid into chamber 10 or removed from chamber 10 to subject patient 82 to elevated temperature and ambient temperature to generate temperature cycles similar to the cycles generated by raising and lower patient 82 using patient support 52.

In the embodiment of FIGS. 2-4, the plurality of legs 72 terminate in feet 86. In a configuration where bed or mattress 84 slides into and out from interior 30 of chamber 10, it can be advantageous to support chamber 10 on feet that resist the movement of chamber 10. However, it should be understood that wheels 74, with brakes on at least two, and preferably all of wheels 74, can provide sufficient resistance to movement to enable the sliding of mattress 84 into and out from interior 30 of chamber 10 while limiting or preventing movement of chamber 10.

Chamber 10 can also include one or more patient temperature sensors 88. One or more patient temperature sensors 88 are configured to interface with Abreu Brain Tunnel Terminuses (ABTTs) 90. ABTTs 90 are discussed in more detail in, for example, U.S. Pat. No. 10,251,776, which is incorporated by reference herein in its entirety.

One or more patient temperature sensors 88 can be supported by, for example, an articulating arm assembly 92. Articulating arm assembly 92 can be fixedly or movably attached to front wall 20 of chamber 10. Articulating arm assembly 92 can include, for example, a first arm 94, which is directly connected to front wall 20. Articulating arm assembly 92 can further include a first rotary pivot 96, a second rotary pivot 98, and a second arm 100. First rotary pivot 96 is positioned at a distal end of first arm 94, which is the opposite end of first arm 94 from where first arm 94 attaches to front wall 20. First rotary pivot 96 provides rotation about an axis that is approximately parallel to a ground plane. Second rotary pivot 98 is attached to first rotary pivot 96, usually directly attached, but a spacer could be positioned between first rotary pivot 96 and second rotary pivot 98. Second rotary pivot 98 provides rotation about an axis that is approximately perpendicular to the rotary axis of first rotary pivot 96. A proximal end of second arm 100 can be directly attached to second rotary pivot 98 or can be attached to second rotary pivot 98 by way of a spacer (not shown). Articulating arm assembly 92 can further include a sensor support 102, which can be directly or indirectly connected to second arm 100 at a proximal end of sensor support 102. Sensor support 102 supports a pair of temperature sensor support arms 104. Arms 104 are connected at a proximal end to a distal end of sensor support 102 and extend from sensor support 102. Each of arms 104 includes a plurality of articulating or movable arms and joints at the end of which is positioned a respective one of patient temperature sensors 88. The function of articulating arm assembly 92 is to enable the positioning of one or more patient temperature sensors 88 on respective ABTTs 90 while patient 82 is positioned in interior 30 of chamber 10. While not shown in FIG. 2, first arm 94 and second arm 100 of articulating arm assembly 92 can include extension features to permit one or more patient temperature sensors 88 to move with patient 82 when patient 82 is moved into and out from interior 30, either vertically or horizontally. Articulating arm assembly 92 may also include a head support to secure patient temperature sensors 88 to the head of patient 82 to enable continuous temperature measurements of patient 82 by way of ABTTs 90.

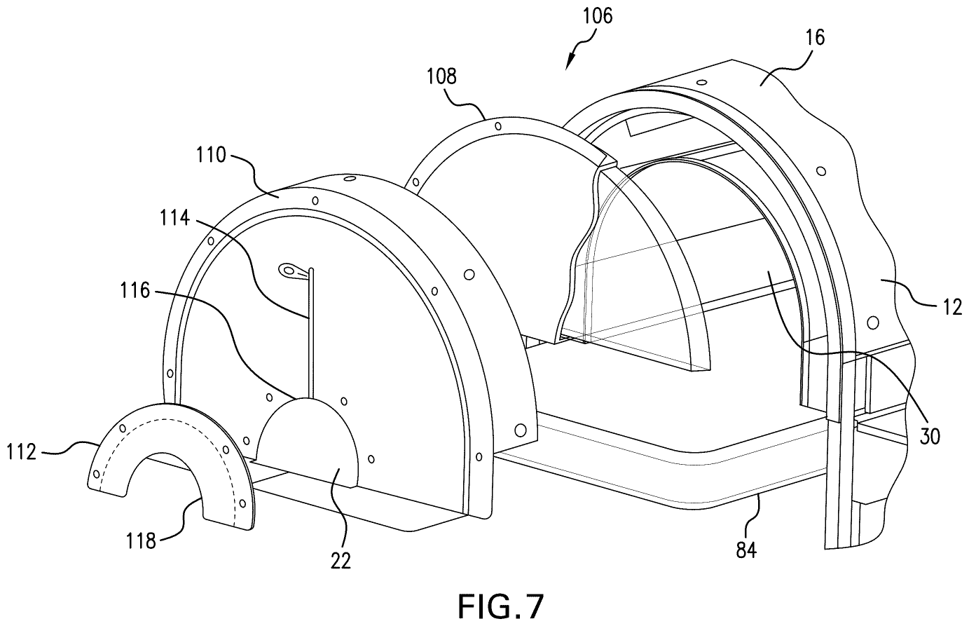

Front wall 20 can be a complex structure that enables a tight fit around neck 68 of patient 82. For example, as shown in FIG. 7, the front wall is a front wall assembly 106 including multiple components. Front wall assembly 106 includes, from interior 30 to exterior 32, an interior insulating layer 108, and exterior cover layer 110. Exterior cover layer 110 also includes insulating properties. Front wall assembly 106 can also include an adapter 112 for providing a close or an exact match with an exterior of neck 68. “Close” in the context of this disclosure means near the neck, which means there can be a gap between adapter 112 and neck 68. Such gap can be several inches. However, the gap is preferably as small as possible. “Exact” in the context of this disclosure means in direct contact with neck 68. It should be understood that even with an “exact” match of neck 68, there may be limited, small gaps between adapter 112 and neck 68.

Interior insulating layer 108 is typically attached directly to, for example, first sidewall 12, second sidewall 14, and top or upper wall 16. Interior insulating layer 108 extends from, for example, top or upper wall 16 to approximately two-thirds of the way from top or upper wall 16 to mattress 84. Interior insulating layer 108 provides insulation to retain heat within chamber 10, particularly in the upper portion of chamber 10. To resist the high or elevated temperatures contained within chamber 10, interior insulating layer 108 can be fabricated from, for example, a silicone-based material or other temperature-resistant insulating materials.

Exterior cover layer 110 is a further layer of insulation that extends from top or upper wall 16 along first sidewall 12 and second sidewall 14 to about the level of mattress 84. To enable ease of entry of patient 82, exterior cover layer 110 can include one or more opening fasteners 114. One or more opening fasteners 114 can be, for example, a hook and loop arrangement, a zipper, snaps, or other mechanism configured to secure parts of exterior cover layer 110 to each other to provide a relatively close fit to neck 68 of patient 82.

It should be noted that while exterior cover layer 110 is shown with an opening 116 that can be approximately the size of a typical neck 68, exterior cover layer 110 can be larger than a typical neck 68 to provide easier entry of patient 82 into and out from interior 30. If opening 116 is larger than a typical neck 68, adapter 112, described in more detail elsewhere herein, is attachable to exterior cover layer 110 to cover whatever remains of opening 116 when one or more opening fasteners 114 are entirely closed to avoid having hot air rushing past neck 68, which could be uncomfortable for patient 82 as well as being inefficient with respect to the operation of temperature therapy system 50. Adapter 112 can come in a plurality of sizes to adapt to different sized necks 68. Adapter 112 includes an opening 118 that is sized for a corresponding size of human neck 68, as described in more detail elsewhere herein. In an alternative embodiment, the material of adapter 112 is flexible to permit neck 68 to stretch adapter 112 to enable close contact of adapter 112 with neck 68 of patient 82, which may also be described as exact contact when adapter 112 touches neck 68, leaving at most a few, small gaps between adapter 112 and neck 68.

FIG. 8 shows an alternative embodiment chamber 130 along with an exemplary cross-section of a wall 132 of chamber 130. Wall 132 can be used with any of the chambers disclosed herein. Wall 132 can include a central insulating layer 134. Central insulating layer 134 can be a plastic, a lightweight insulating ceramic, or other heat-insulating material that can withstand temperatures internal to chamber 130 with minimal or no deterioration over time. Positioned directly between central insulating layer 134 and exterior 32 is an external shell 136. Since central insulating layer 134 prevents elevated temperatures from reaching external shell 136, external shell 136 can be a plastic material with lower temperature resistance than the materials adjacent to interior 30 of chamber 130. Such plastic material can be hard and scratch-resistant to resist damage when moving chamber 130 from one location to another, and to resist damage from the proximity of transport beds, medical equipment, etc., to chamber 130 when being operated by medical personnel.

Wall 132 can also include a first gas layer 138 positioned between central insulating layer 134 and external shell 136. First gas layer 138 provides additional insulation of external shell 136 from the heat of interior 30 since the trapped gas of first gas layer 138 resists the transmission of heat. First gas layer 138 can be an inert gas such as argon, krypton, and/or xenon, or first gas layer 138 can be atmospheric gas or nitrogen, depending on the insulating capabilities of the layers of central insulating layer 134.

Wall 132 can also include an interior layer 140. The material of interior layer 140 is preferably a low thermal conductivity material that exists to prevent damage to the internal structure of wall 132 as well as to limit or prevent heat transfer to patient 82 should patient 82 unintentionally contact wall 132. For example, interior layer 140 can be a silicone material. An optional inner insulating wall 142 can be directly positioned between central insulating layer 134 and interior layer 140. A second gas layer 144 can be positioned directly between central insulating layer 134 and interior layer 140, and if optional inner insulating wall 142 is present, a third gas layer 146 can be positioned directly between optional inner insulating wall 142 and interior layer 140. Each of second gas layer 144 and third gas layer 146 can be any of the gases used for first gas layer 138.

FIG. 9 shows another temperature therapy system 150 of the present disclosure. Temperature therapy system 150 includes a chamber 152, a power supply 154, a computer 156 with internal processor and non-transitory computer-readable medium, which are connected to each other either physically or wirelessly, for controlling temperature therapy system 150, a control panel 158 for operating temperature therapy system 150, and an antenna 160 to enable transmission of data to and from a remote server or controller. Antenna 160 can also enable remote operation of temperature therapy system 150 by a medical professional. To minimize wires and connections, chamber 152 can communicate with computer 156 and/or control panel 158 through a transmitter 162.

Temperature therapy system 150 also includes a patient support 164. Patient support 164 includes a leg or stand 166 at a distal end away from chamber 152. Stand 166 supports both patient support 164 and patient 82 when patient 82 is positioned on patient support 164. Stand 166 can include rollers or wheels (not shown) to aid in sliding patient support 164 by way of slides 168 internal to chamber 152 in combination with the rollers or wheels. Patient support 164 or stand 166 can include a handle 170 to add in moving patient support 164. Note that chamber 152 includes a front wall assembly, which can be similar to front wall assembly 106, but the front wall assembly is removed in FIG. 9 to simplify the drawing.

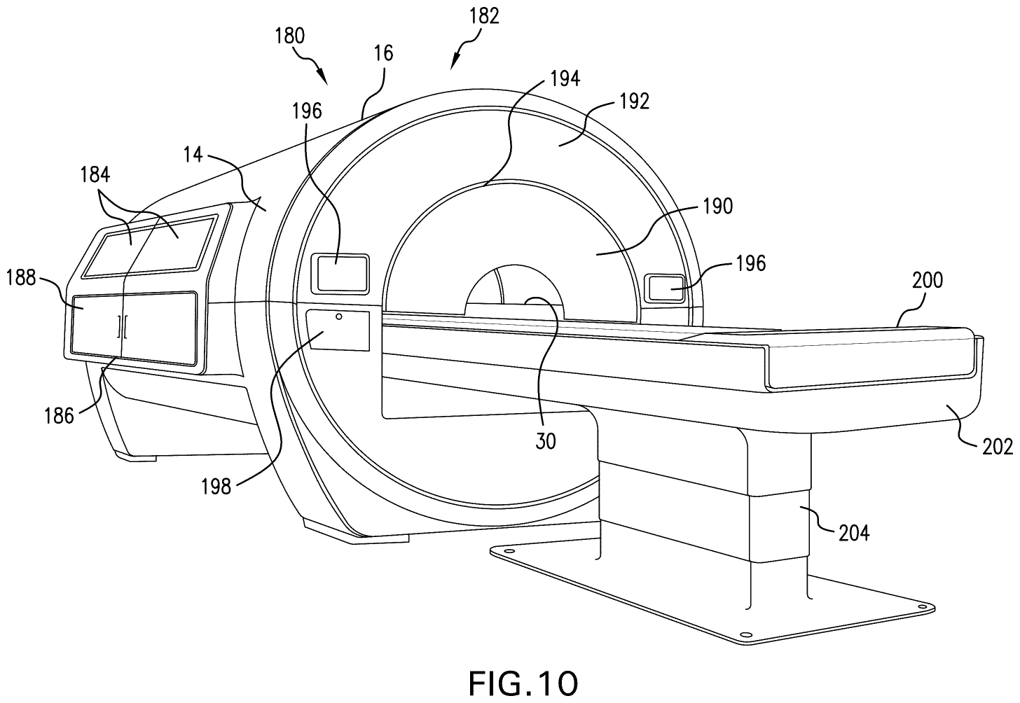

FIG. 10 shows a temperature therapy system 180 that includes integrated controls. More specifically, temperature therapy system 180 includes a chamber 182 that includes internal power distribution (not shown), display screens 184, and internal computer with a processor operated by way of a control station 186. The processor is expected to be in a physical or wireless connection with a non-transitory computer-readable medium, as described elsewhere herein. The non-transitory computer-readable medium can contain the software that is transmitted to the processor that the processor uses to operate temperature therapy system 180. Control station 186 may be covered by panels 188 which can be locked to prevent access to control station 186. Display screens 184 can be movable to enable an operator to move near head 83 or neck 68 of patient 82 while monitoring display screens 184. Chamber 182 includes a retractable heat seal 190 that can be raised into and lowered from a slot 194 internal to a front wall 192.

Chamber 182 can also include optional touchscreens 196 that enable, for example, control of some functions provided by display screens 184, and display of some functions of display screens 184, particularly when a medical practitioner is positioned close to patient 82.

Chamber 182 can include other features. For example, chamber 182 can include a sensor compartment 198 to store, for example, temperature sensors for placement on patient 82.

Temperature therapy system 180 includes a patient support 200 and a patient support cradle 202. Patient support 200 is slidably positioned on patient support cradle 202 and patient support cradle 202 is supported by an exterior stand 204 that includes an integral base. Patient support cradle 202 extends into interior 30 so that patient support cradle 202 can move patient 82 into and out of interior 30 to enable rapid exposure and removal from the elevated temperatures in interior 30.

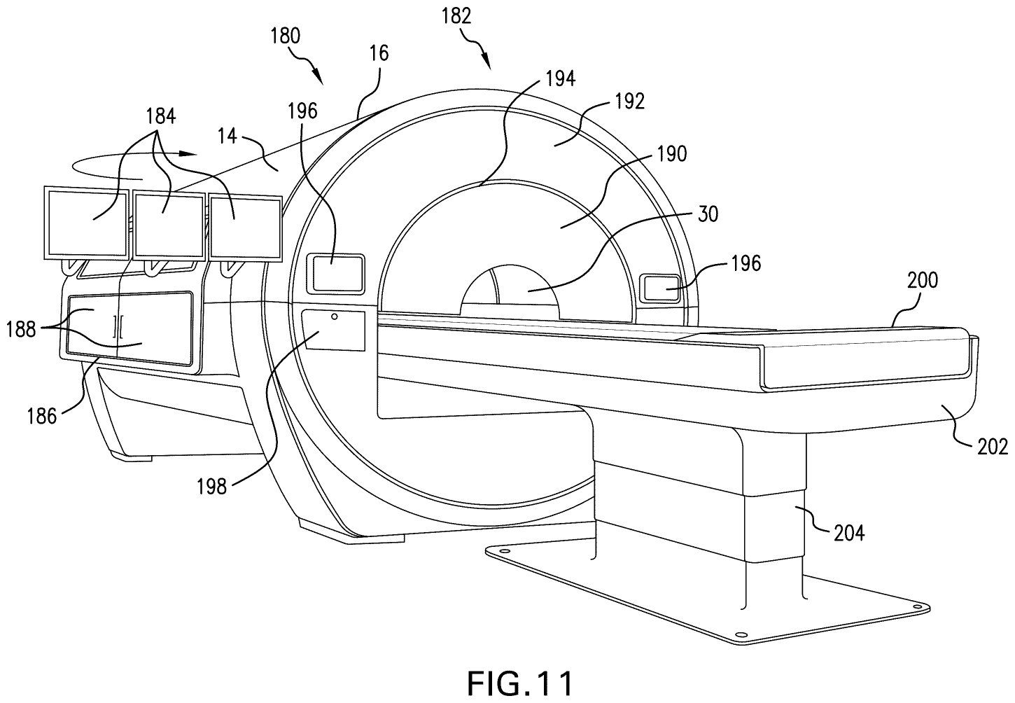

FIG. 11 shows a perspective view of temperature therapy system 180 with display screens 184 rotated toward a patient entry end of chamber 182.

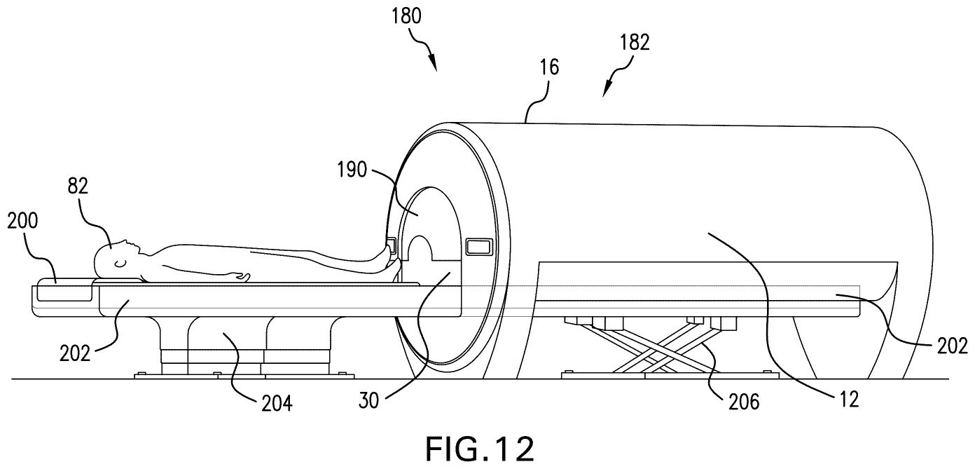

Exterior stand 204 can also be configured to raise and lower patient 82 for ease of entry into chamber 182. In the configuration of FIG. 12, chamber 182 includes a second, interior cradle 202 and an interior stand 206. Exterior stand 204 and interior stand 206 can move up and down vertically simultaneously when moving patient 82 from exterior cradle 202 to interior cradle 202. Patient support 200 in FIG. 12 when positioned on exterior stand 204 is fully supported by exterior cradle 202 and exterior stand 204. Patient support 200 can then be moved on slides, rollers, or by other hardware from exterior cradle 202 and exterior stand 204 to interior cradle 202 and interior stand 206 to be fully supported by interior stand 206.

Exterior stand 206 and interior stand 206 can be configured as shown in, for example, FIG. 10. However, interior stand 206 shown in FIG. 12 is a conventional scissor-type stand that can also be used to support interior cradle 202. It should be noted that the scissor configuration shown in FIG. 12 can be used with most of the temperature modification system embodiments disclosed herein.

In an embodiment, exterior stand 204 and interior stand 206 are lowered before moving patient 82 from exterior stand 204 to interior stand 206. After patient support 200 is moved from exterior stand 204 to interior stand 206, patient support 200 and interior cradle 202 are raised with patient 82 thereon to position patient 82 into interior 30, the location where an elevated temperature is present for thermal treatment of patient 82. To cycle temperature exposure of patient 82, in an embodiment interior stand 206 can be moved rapidly and independently of exterior stand 204 in an up and down or vertical direction to quickly subject patient 82 to elevated temperatures and to then remove patient 82 quickly from elevated temperatures. To remove patient 82 from treatment, interior stand 206 and exterior stand 204 are lowered simultaneously. Patient support 200 is then moved from interior cradle 202 to exterior cradle 202. Patient 82 can then be raised or lowered by exterior stand 204 to position patient 82 for transfer to a movable hospital bed, to position patient 82 for movement from patient support 200 to the floor adjacent to exterior stand 204, or to position patient 82 for movement from patient support 200 to another location, such as a wheelchair (not shown).

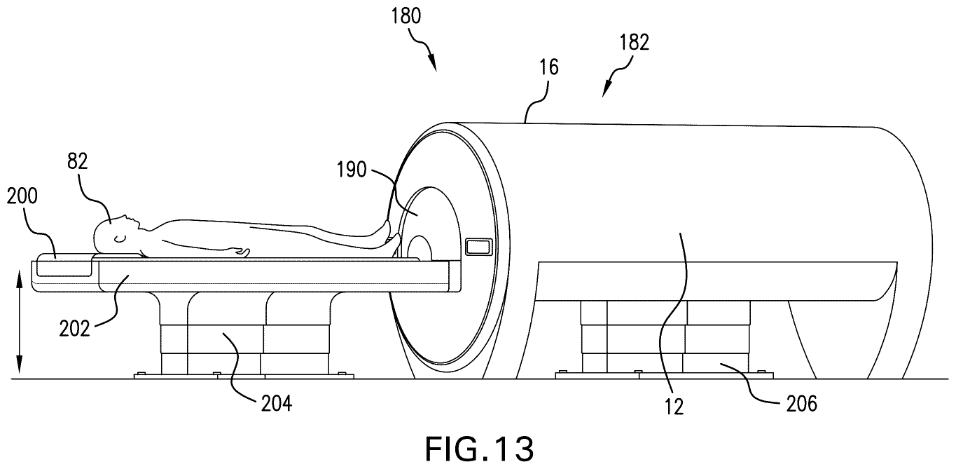

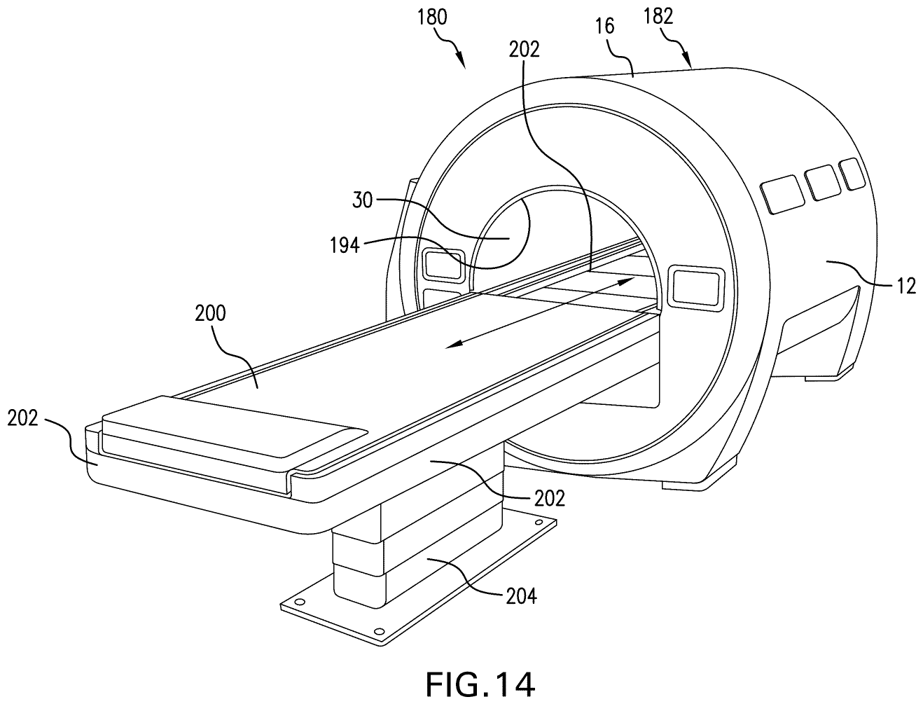

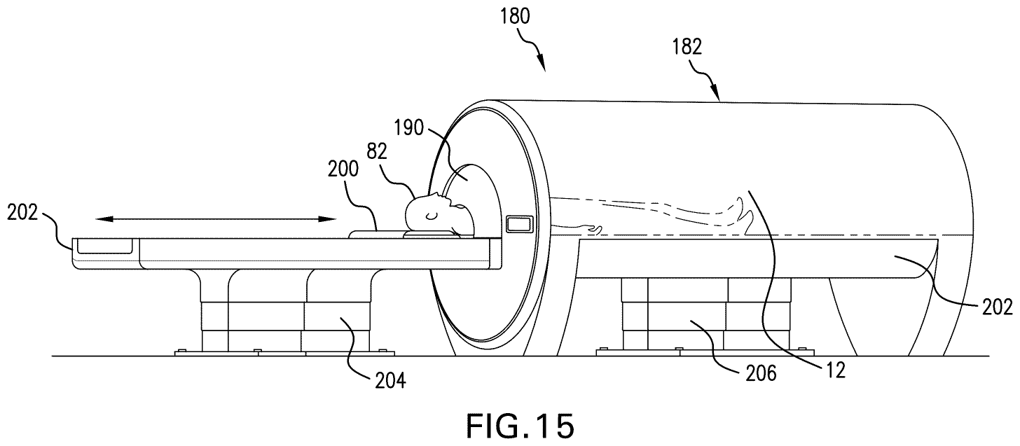

FIG. 12 shows exterior stand 204 and interior stand 206 in a lowered position, and FIG. 13 shows exterior stand 204 and interior stand 206 in a raised position. FIG. 14 shows a configuration of chamber 182 where retractable heat seal 190 is either retracted into slot 194 or heat seal 190 can be configured to be removed entirely to enable patient support 200 and patient 82 to be moved from exterior cradle 202 to internal cradle 202 along rails or slides. FIG. 15 shows the embodiment of FIG. 14 with patient 82 positioned in interior 30 and retractable heat seal 190 placed into position adjacent to neck 68.

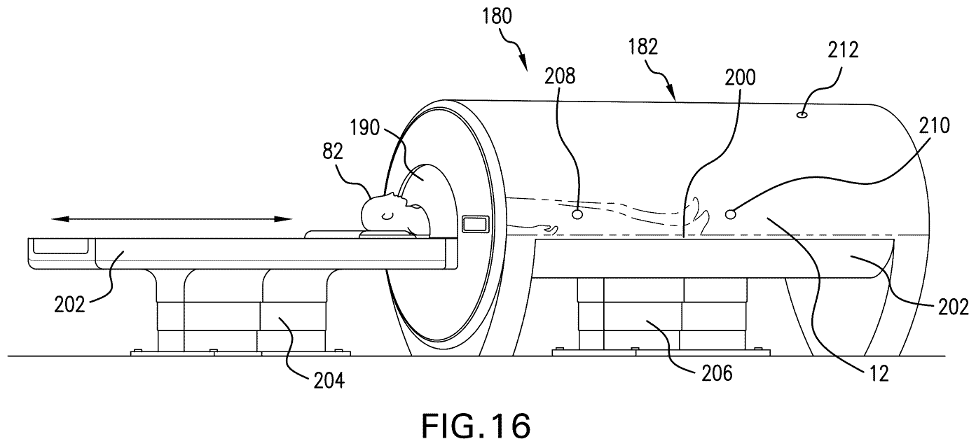

FIG. 16 shows the temperature therapy system 180 of FIG. 14 with temperature sensors 208 and 210 positioned at a position closer to patient support 200 than to top or upper wall 16. Temperature sensors 212, for example, 24 temperature sensors 212 can extend along a longitudinal length of chamber 182 on the top interior of chamber 182 to measure temperature applied to patient 82 along a length of patient 82. Temperature sensors 208 and 212 can also be configured similarly to the arrangement of temperature sensors 62 shown in FIG. 1. Temperature sensors 208, 210, and 212 enable integration of the temperature profile applied to patient 82 to verify that the amount of heat applied to patient 82 is appropriate for a specific treatment.

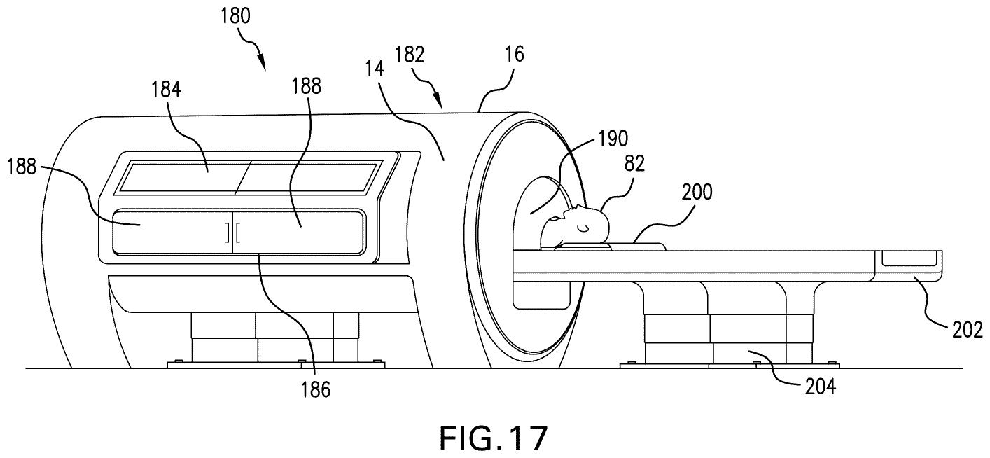

FIG. 17 shows another perspective view of chamber 182 showing the placement of display screens 184. Display screens 184 can be, for example, 13-inch to 16-inch monitors extending in a line or row longitudinally along chamber 182 when stored or retracted. As disclosed herein, display screens 184, which are positioned on second sidewall 14 in FIG. 17, are positioned on an upper half of second sidewall 14. Display screens 184 can be swung or rotated toward 204, enabling a medical practitioner to access display screens 184 while being positioned adjacent to patient 82 when patient 82 is positioned on exterior stand 204.

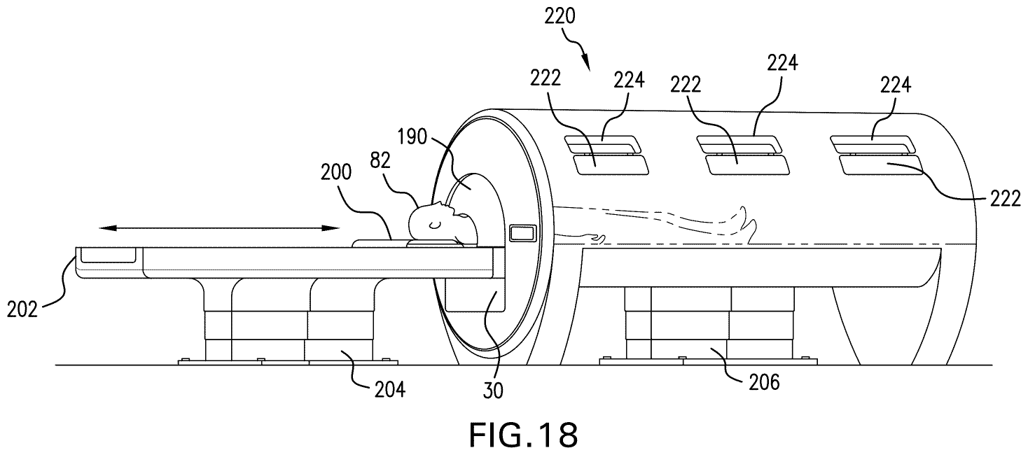

FIG. 18 shows a temperature therapy system 220 in accordance with an exemplary embodiment of the present disclosure. Temperature therapy system 220 includes a plurality of vents 224 covered by downwardly opening vent doors 222. Vents 224 can include high-speed fans (not shown) to evacuate heated air from interior 30 rapidly, which can be for emergencies, or which can aid in controlling temperature exposure of patient 82. Downwardly opening vent doors 222 can be operated manually or can be operated with a mechanism controllable by, for example, computer 156 shown in FIG. 9, display screens 184, optional touchscreens 196, or other control apparatus.

FIGS. 19A and 19B show partially sectioned views of a chamber 230 in a still further exemplary embodiment of the present disclosure. Chamber 230 includes a cylindrical wall 232 with a thick layer of insulation 234. Patient support 200 is movable from exterior 32 to interior 30 along rails 236. Chamber 230 includes at least one infrared heater 238 positioned at a distal end 240 of interior 30. In addition, there can be a plurality of infrared heaters 238 extending horizontally along a longitudinal length of chamber 230 and a plurality of at least one infrared heater 238 extending vertically along cylindrical wall 232 from a location near to where patient 82 is located when patient support 200 is positioned in interior 30 to a top interior of cylindrical wall 232. In this embodiment, exterior stand 204 and interior stand 206 are raised simultaneously, and patient support cradle 202 is contiguous from exterior 32 to interior 30. Chamber 230 includes a front wall 242. Front wall 242 includes a lower opening 244 which provides clearance to patient support cradle 202 to be lowered to distance patient 82 from the hot zone in interior 30 which is the upper region of interior 30. Chamber 230 includes side openings 246 through which airflow 248 passes to prevent the buildup of pressure in interior 30, and to enable rapid temperature decrease when power is removed from at least one infrared heater 238.

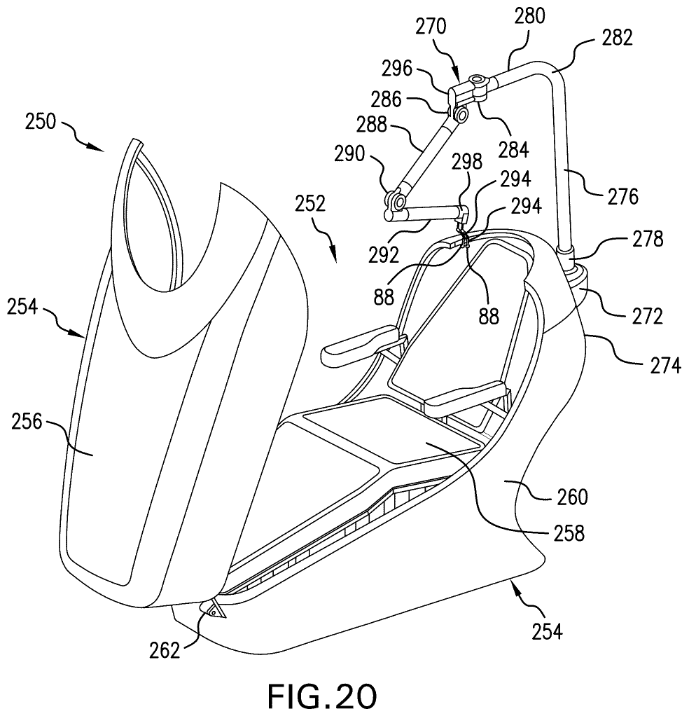

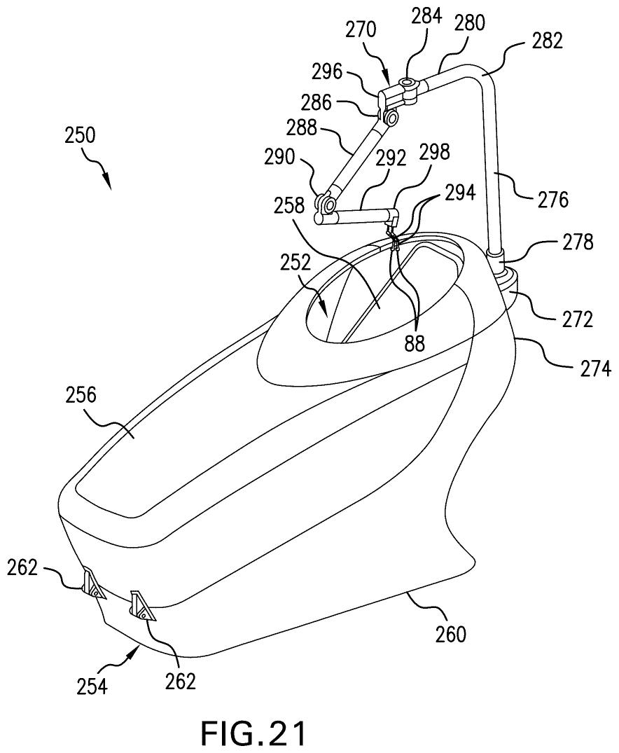

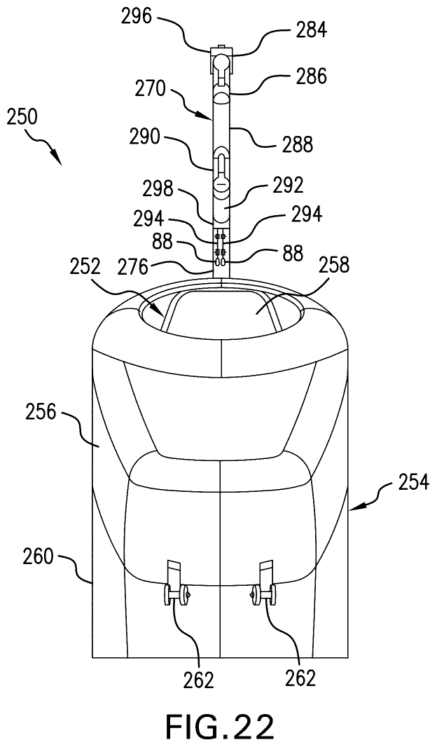

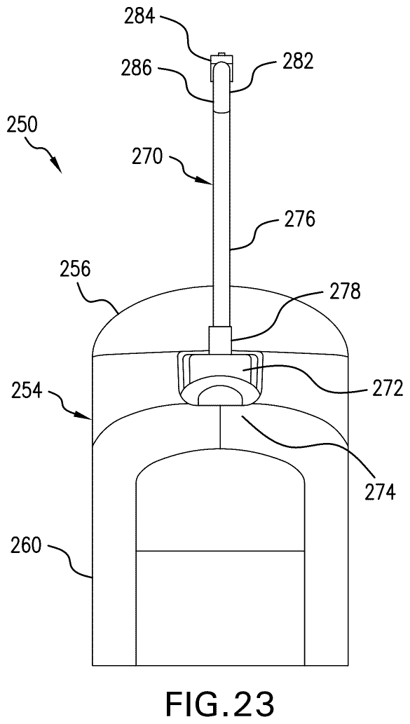

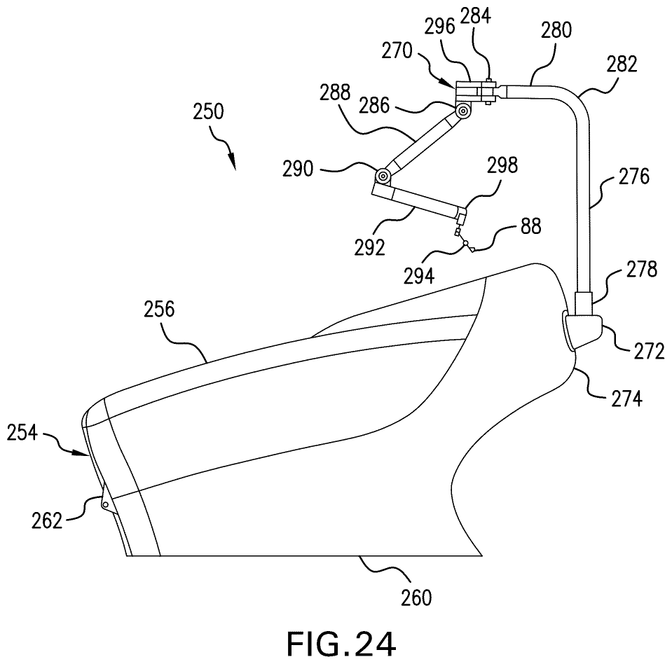

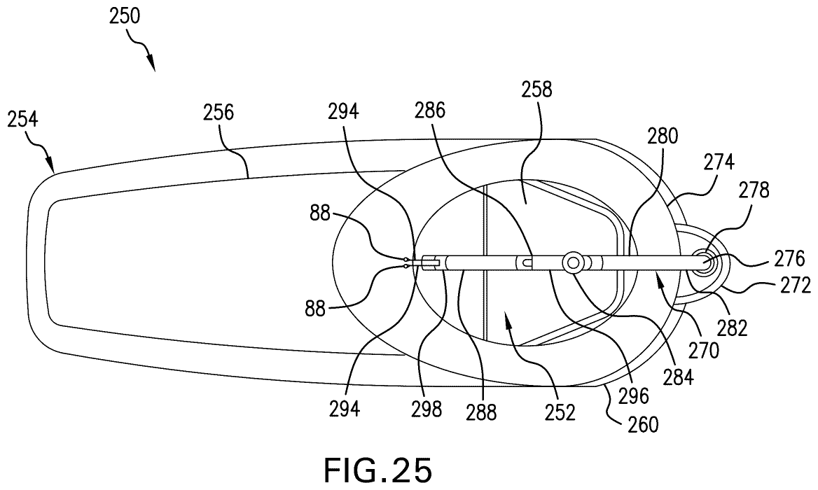

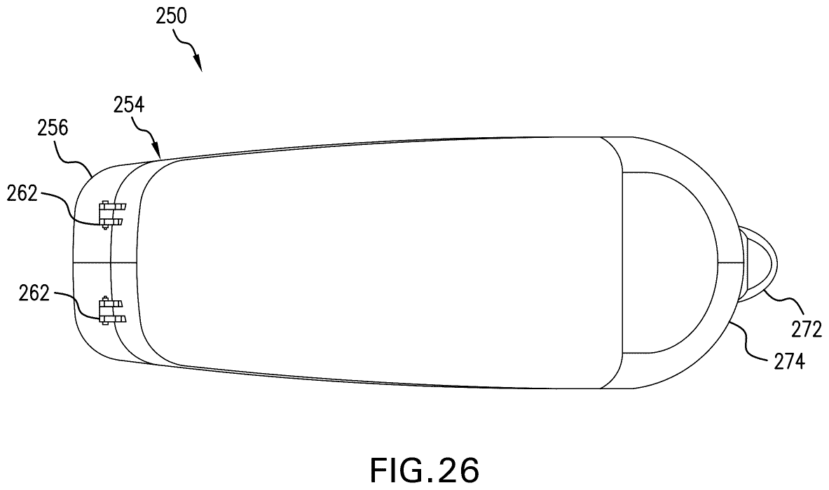

FIGS. 20-26 show a temperature therapy system 250 in accordance with an exemplary embodiment of the present disclosure. Temperature therapy system 250 is advantageous in that internal volume 252 is relatively small so modifying the temperature can be accomplished relatively quickly. Temperature therapy system 250 includes a chamber 254, a pivoting door 256 that is part of chamber 254 that enables access to internal volume 252, and a patient support or seat 258. Chamber 254 also includes a base 260 to which pivoting door 256 is attached at a proximal end by way of pivots 262. Internal to chamber 254 are heaters or coolers (not shown) to enable applying temperature cycles to patient 82.

In FIGS. 20-26, one or more patient temperature sensors 88 can be supported by, for example, an articulating arm assembly 270. Articulating arm assembly 270 can be fixedly or movably attached to a protruding support 272 positioned on a distal end 274 of base 260. Articulating arm assembly 270 includes a first arm 276 attached to protruding support 272 by way of an interface 278. Interface 278 can be a fixed interface. Alternatively, interface 278 can rotate to enable rotation of articulating arm assembly 270 about an axis extending through a center of interface 278. Articulating arm assembly 270 also includes a second arm 280 that is attached to first arm 276 by way of a curved interface 282.

Articulating arm assembly 270 further includes a first rotary pivot 284, a second rotary pivot 286, a third arm 288, a third rotary pivot 290, a fourth arm 292, a pair of sensor support arms 294, and a sensor support 298. First rotary pivot 284 rotates or pivots about an axis that extends approximately vertically. Approximately vertically in the context of articulating arm assembly 270 can be within 25 degrees of vertical. Second rotary pivot 286 is connected to first rotary pivot 284 by a pivot support arm 296. Second rotary pivot 286 rotates or pivots about an axis that extends approximately horizontally. Approximately horizontally in the context of articulating arm assembly 270 can be within 25 degrees of horizontal. Third arm 288 extends from second rotary pivot 286. Third rotary pivot 290 also pivots or rotates about an axis that extends approximately horizontally. Fourth arm 292 is connected to and extends from third rotary pivot 290. Positioned at a distal end of fourth arm 292 is sensor support 298. Extending from sensor support 298 is a pair of support arms 294. Each of support arms 294 includes a plurality of articulating or movable arms and joints at the end of which is positioned a respective one of patient temperature sensors 88. The function of articulating arm assembly 270 is to provide flexibility in positioning one or more patient temperature sensors 88 on ABTTs 90 of patient 82 while patient 82 is positioned in internal volume 252 of temperature therapy system 250. While not shown in FIGS. 20-26, the arms of articulating arm assembly 270 can include extension features to further increase the flexibility to interface one or more patient temperature sensors 88 with patient 82. Articulating arm assembly 270 may also include a head support to secure patient temperature sensors 88 to the head of patient 82 to enable continuous temperature measurements of patient 82 by way of ABTTs 90.

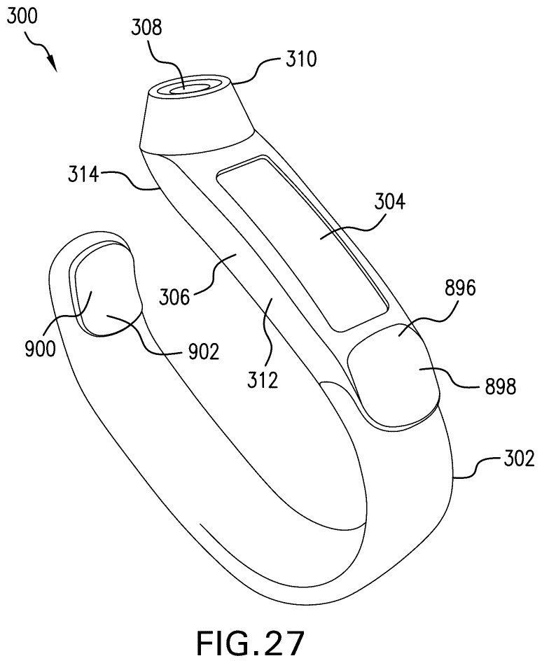

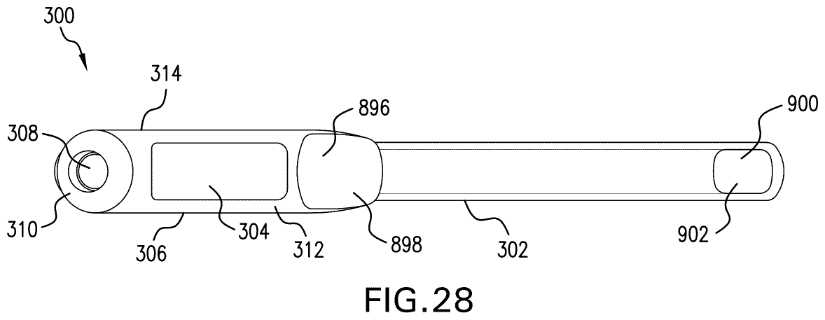

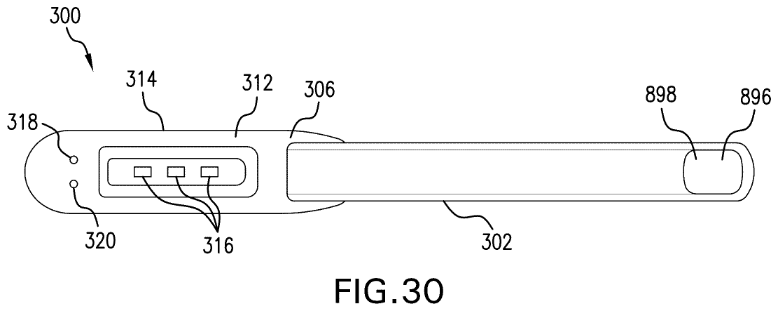

It can be advantageous to apply heat directly to ABTTs 90. Accordingly, a smartwatch 300 as shown in FIGS. 27-30 can perform such a function. Smartwatch 300 includes a band 302, a display 304, which can be used to display temperature and other information, a processor 306 positioned internal to an electronics housing 314, a heat inductor 308 surrounded by an insulating annular interface 310, and a transmitter 312 for wireless communication with, for example, a mobile communication device such as a computer, a tablet, a cellular phone, and the like. Smartwatch 300 can also communicate with, for example, computer 48 by way of antenna 66, or control station 186. Annular interface 310 may be oriented at an angle with respect to a longitudinal extent of band 302 to enable ease of interface with ABTTs 90. Such an angle with respect to the longitudinal extent of band 302 can be, for example, 25 to 45 degrees. Electronics housing 314 can include controls 316 positioned on the backside that control, for example, start of a heat application, stop of heat application, volume, on/off operation, and the like. Electronics housing 314 can also include a speaker 318 to provide alerts, including, for example, excessive temperature of patient 82 alert or malfunction of smartwatch 300. Smartwatch 300 can also interface with a human voice, which is enabled by a microphone 320 that can be positioned on electronics housing 314.

Smartwatch 300 can also include a humidity sensor 896 and an ambient temperature sensor 898 positioned on an exterior surface of band 302. Humidity sensor 896 and ambient temperature sensor 898 can provide information regarding environmental temperature conditions that can be used to modify the heat provided by heat inductor 308. For example, in high-humidity, high-temperature situations, heat inductor 308 may need to apply a higher temperature to ABTT 90 to compensate for an increased ambient temperature at ABTT 90 and a decreased evaporation rate.

Smartwatch 300 can also include a wearer temperature sensor 902 positioned on an interior of band 302 to enable direct contact with patient 82 to measure the temperature of patient 82. Smartwatch 300 can further include a sweat sensor 900 positioned on an interior surface of band 302 to be in direct contact with patient 82 to enable measurement of the sweat emitted by patient 82. As with humidity sensor 896 and ambient temperature sensor 898, sweat sensor 900 and wearer temperature sensor 902 can be used to modify the heat from heat inductor 308 applied to ABTT 90.

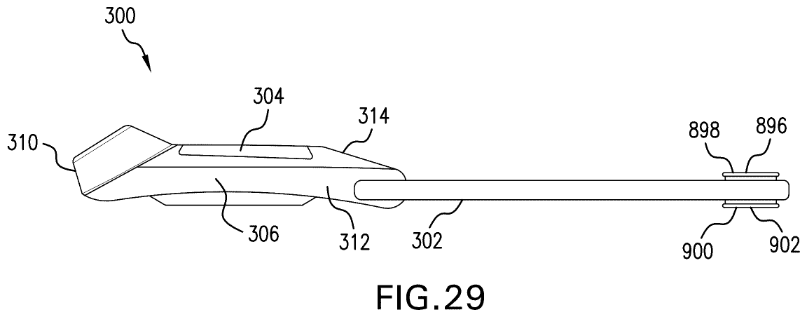

FIG. 29 shows a slightly different embodiment of smartwatch 300. More specifically, sweat sensor 900 and wearer temperature sensor 902 are positioned at a first end of band 302 on an opposite side of band 302 from humidity sensor 896 and ambient temperature sensor 898. Thus, all sensors positioned on smartwatch 300 are collocated with each other at the first end of band 302. In this embodiment, electronics housing 314 is positioned at a second, opposite end of band 302 from humidity sensor 896, ambient temperature sensor 898, sweat sensor 900, and wearer temperature sensor 902.

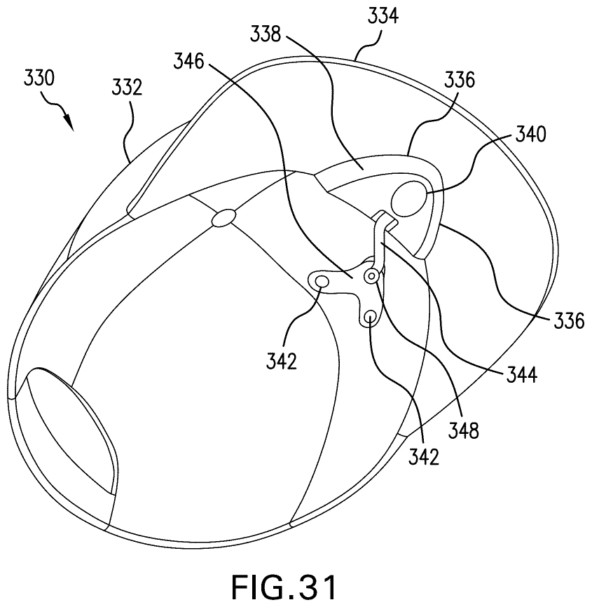

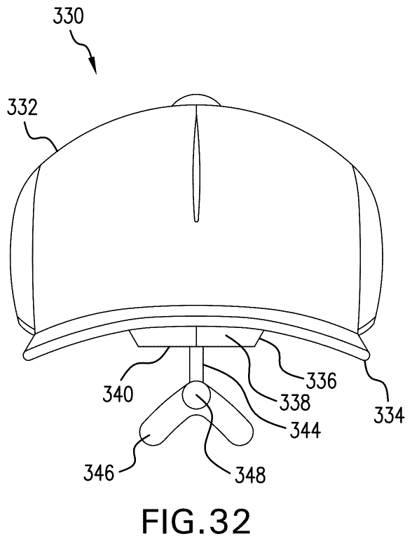

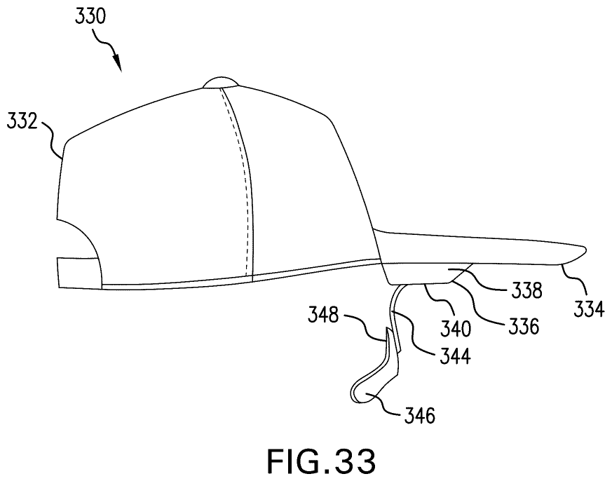

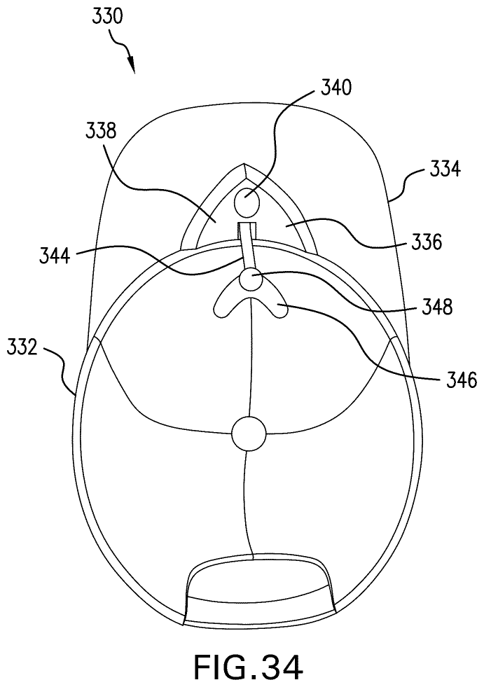

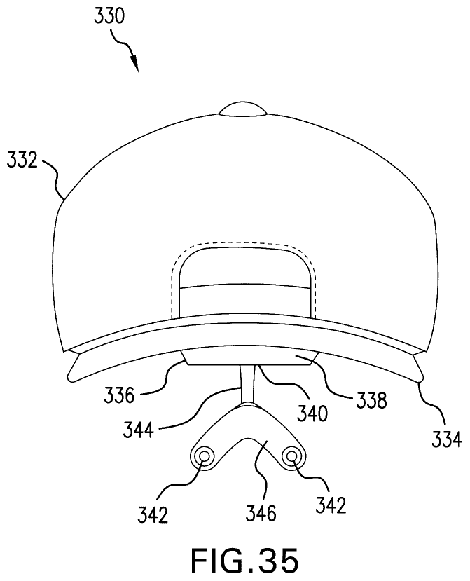

FIGS. 31-35 show a hat 330 in accordance with an exemplary embodiment of the present disclosure. Hat 330 can perform a function similar to smartwatch 300 described herein. Hat 330 includes a hat body 332, and a bill 334 extending from a front of hat body 332. Bill 334 can be semi-rigid to support an electronics housing 336. Internal to electronics housing 336 is a processor 338 and a transmitter 340 for wireless communication with, for example, a mobile communication device such as a computer, a tablet, a cellular phone, and the like. Smartwatch 300 can also be in communication with, for example, computer 48 by way of antenna 66, or control station 186. Electronics housing 336 can include an inductor arm 344 extending therefrom, which terminates at a heat inductor support 346 at a distal end thereof. On heat inductor support 346 is positioned a pair of heat inductors 342 that are sized and dimensioned to interface with ABTTs 90 of patient 82. Inductor arm 344 can be flexible to enable adjustment of the position of heat inductors 342 to contact ABTTs 90 of patient 82. A pivot 348 can be positioned directly between inductor arm 344 and heat inductor support 346 to provide additional adjustability of the position of heat inductors 342.

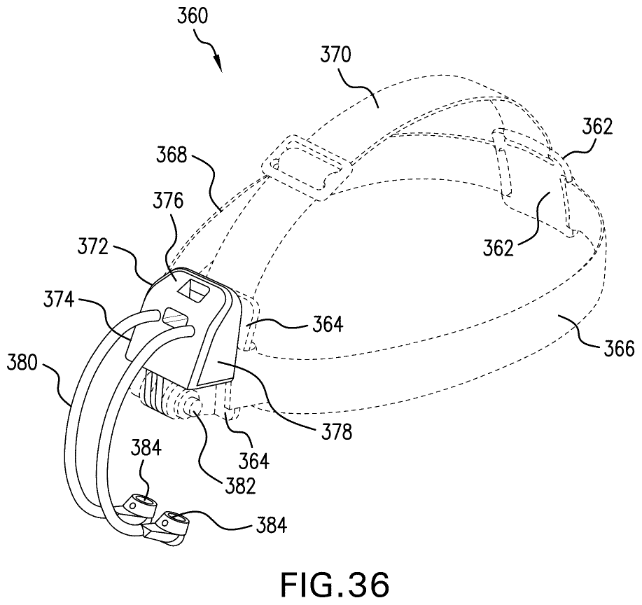

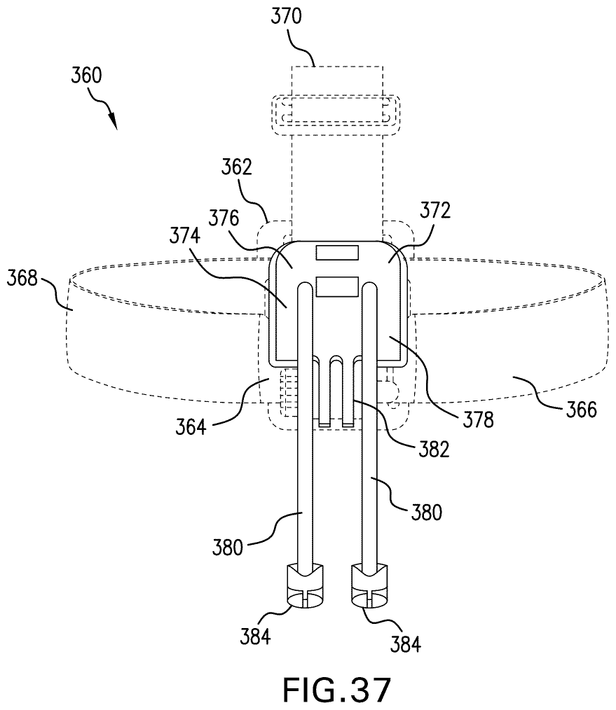

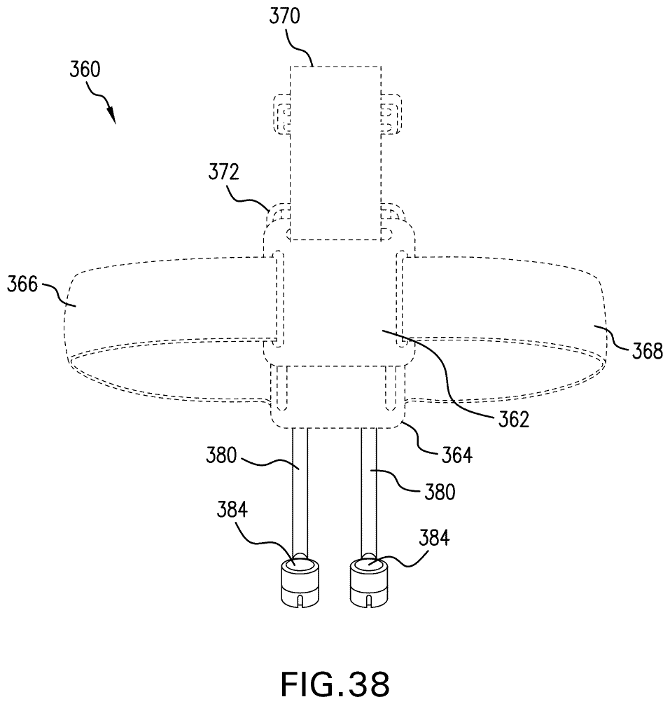

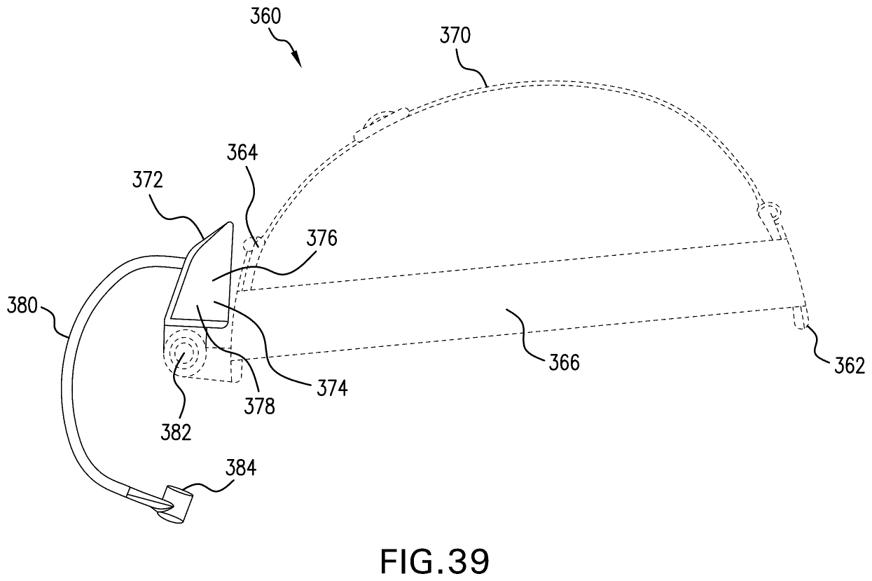

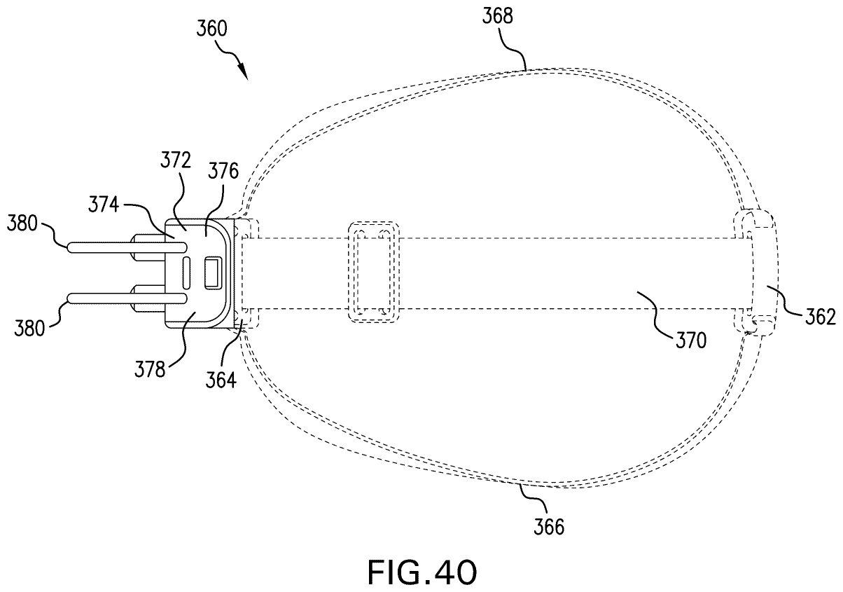

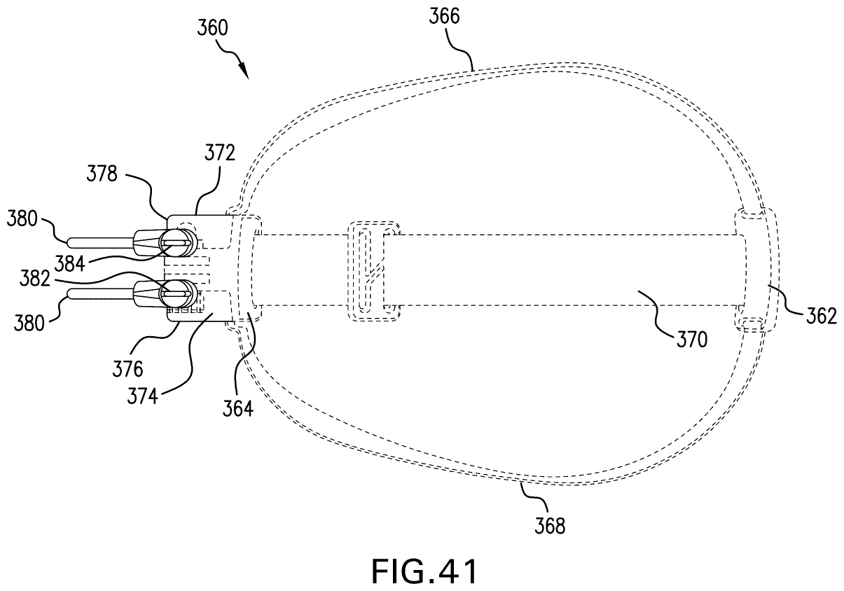

FIGS. 36-41 show a head support 360 in accordance with an exemplary embodiment of the present disclosure. Head support 360 can perform a function like smartwatch 300 and hat 330 described herein. Head support 360 includes a first joint 362, a second joint 364, a first strap 366, a second strap 368, a third strap 370, and an electronics housing 372. Each of first strap 366, second strap 368, and third strap 370 can stretch to fit a heat of patient 82, or can be adjustable via known techniques. A distal end of each of first strap 366, second strap 368, and third strap 370 is attached to first joint 362, and a proximal or front end of each of first strap 366, second strap 368, and third strap 370 is attached to second joint 364 to form a framework for being supported by the head of patient 82. Electronics housing 372 is attached to second joint 364 by way of an electronics housing pivot 382 which enables rotation of electronics housing 372.

Electronics housing 372 includes a processor 374, a transmitter 376, which can communicate with the various systems described herein without limit, and a power supply 378. Electronics housing 372 supports a pair of inductor support arms 380, which are attached to electronics housing 372 at a proximal end of Inductor support arms 380. Inductor support arms 380 can be flexible. Positioned at a distal end of each of inductor support arms 380 is an inductor 384. Each inductor 384 is configured to mate with an associated ABTT 90 of patient 82. As with hat 330, inductors 384 can be commanded by way of transmitter 376 to actuate power supply 378 to provide power to inductors 384 to apply heat to ABTTs 90. Electronics housing 372 is rotatable by way of electronics housing pivot 382 to enable movement of inductor 384 downwardly to provide repositioning of inductors 384 to better adapt to ABTTs 90.

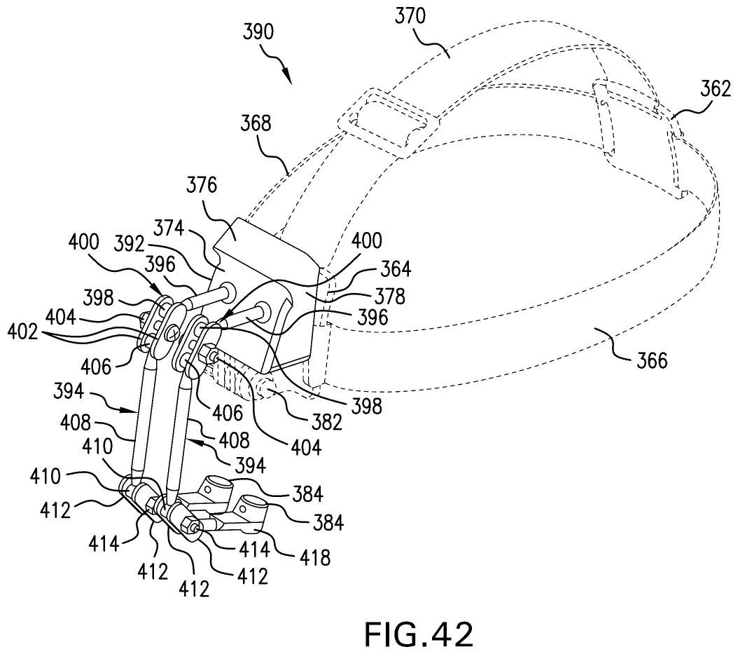

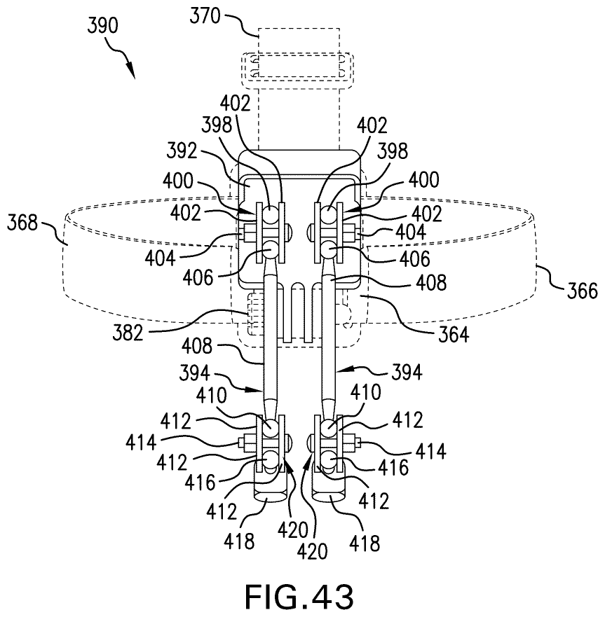

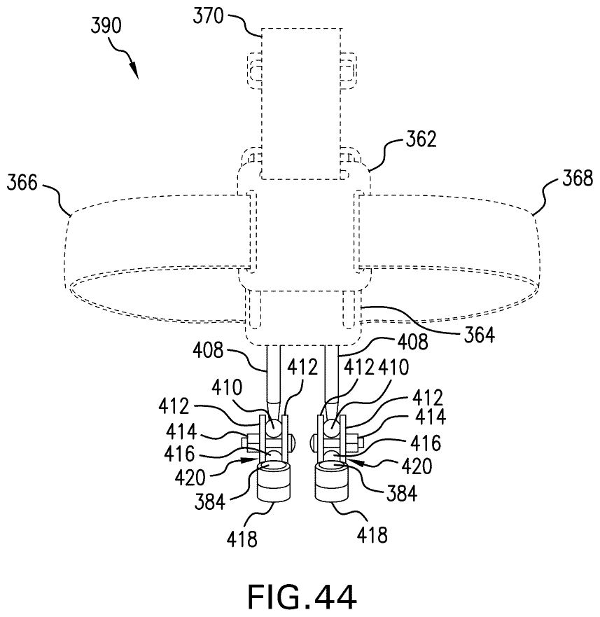

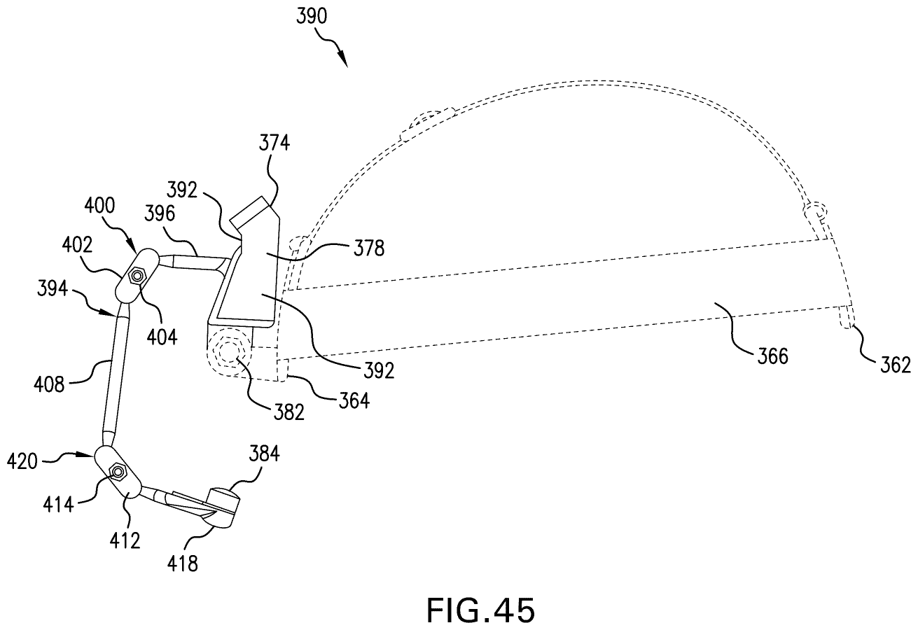

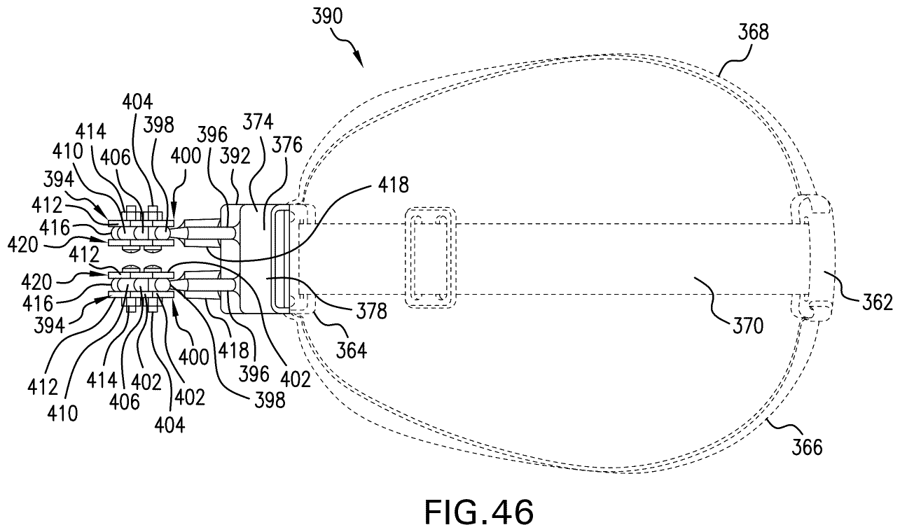

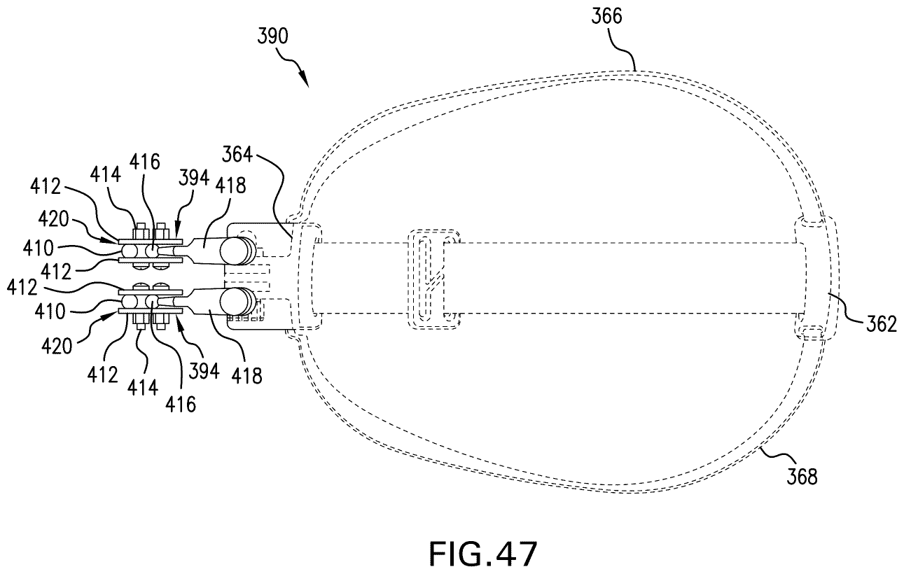

FIGS. 42-47 show a head support 390 in accordance with an exemplary embodiment of the present disclosure. Head support 390 can perform a function like smartwatch 300, hat 330, and head support 360 described herein. Head support 390 includes features that may be similar or identical to head support 360. Accordingly, such features are provided with the same reference number as that used on head support 360, and only those parts that are unique to head support 390 are described.

Head support 390 includes an electronics housing 392. Electronics housing 392 is attached to second joint 364 by way of electronics housing pivot 382 which enables rotation of electronics housing 392. Electronics housing 392 includes a processor 374, a transmitter 376, which can communicate with the various systems described herein without limit, and a power supply 378.

Electronics housing 392 supports a pair of inductor support arm assemblies 394, each of which is attached to electronics housing 392 at a proximal end of inductor support arm assemblies 394. Inductor support arm assemblies 394 include multiple arms movably connected to each other. More specifically, each inductor support arm assembly 394 includes a first arm 396 fixedly attached to electronics housing 392. A distal end of first arm 396 includes a first ball pivot 398. First ball pivot 398 connects to a pivot link assembly 400. Pivot link assembly 400 includes a pair of plates 402 that are attached to each other by a fastener 404. Fastener 404 is tightened to clamp first ball pivot 398 such that frictional force is applied to first ball pivot 398, thus securing the orientation of plates 402. Pivot link assembly 400 includes a second ball pivot 406 that is also clamped by plates 402. Second ball pivot 406 is attached to a second arm 408. When second ball pivot 406 is clamped by plates 402, the frictional force on second ball pivot 406 maintains the orientation of second arm 408. At a distal end of second arm 408 from second ball pivot 406 is a third ball pivot 410 that is fixedly attached to the distal end of second arm 408.

Similar to second ball pivot 406, third ball pivot 410 is clamped by a pair of plates 412 that are attached to each other by a fastener 414 as part of a pivot link assembly 420. When fastener 414 is tightened, plates 412 provide frictional force to third ball pivot 410 to restrict the movement of third ball pivot 410. Plates 412 also provides frictional force to a fourth ball pivot 416, which is also part of pivot link assembly 420. Fourth ball pivot 416 is attached to an inductor support arm 418 which supports inductor 384. The combination of first ball pivot 398, second ball pivot 406, third ball pivot 410, and fourth ball pivot 416 enables the three-dimensional positioning of each inductor 384 to mate with the corresponding ABTTs 90 of patient 82.

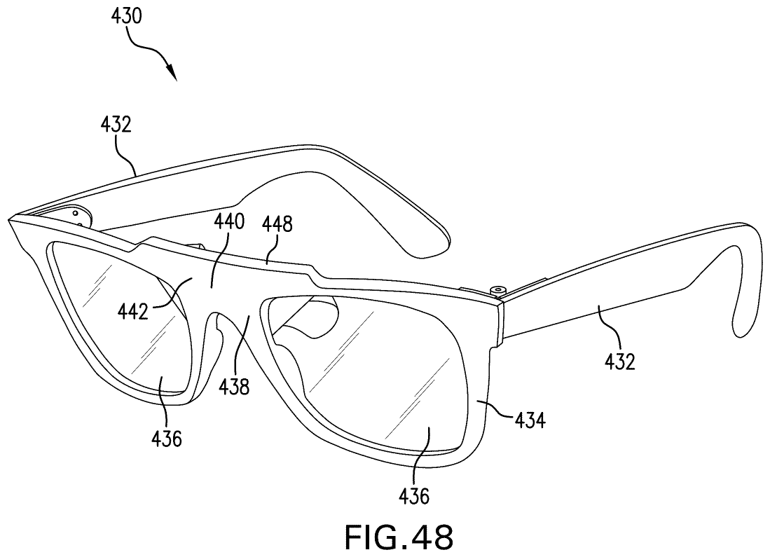

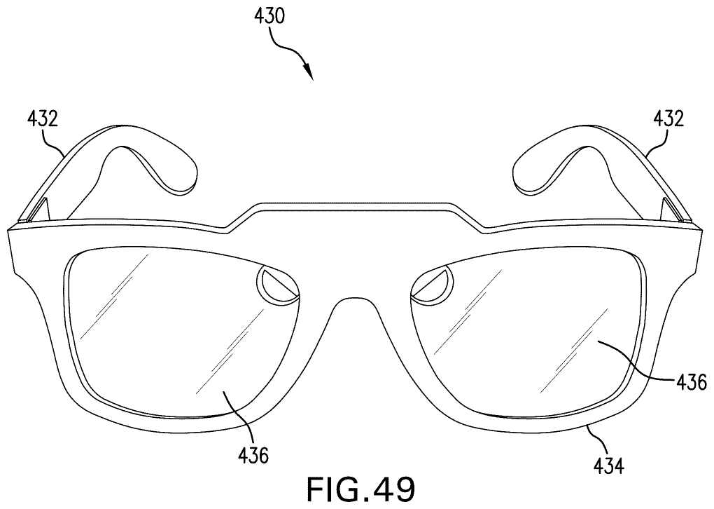

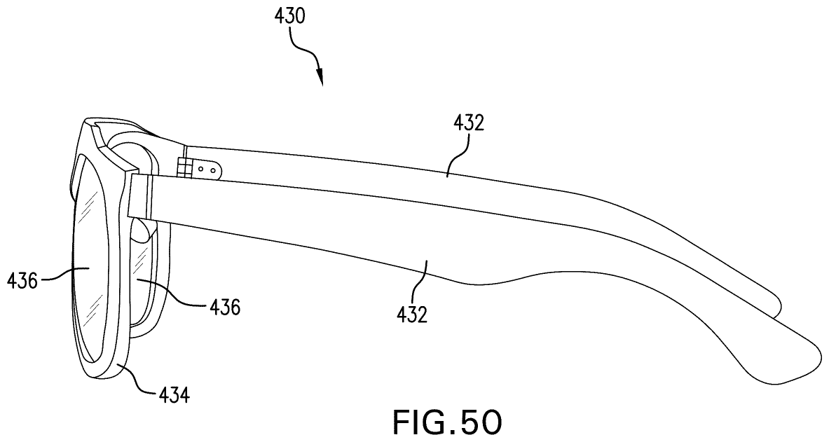

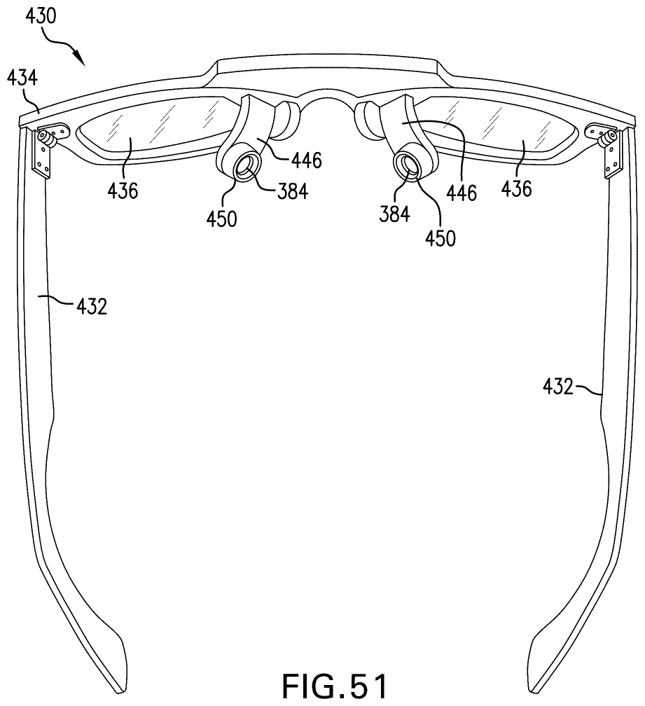

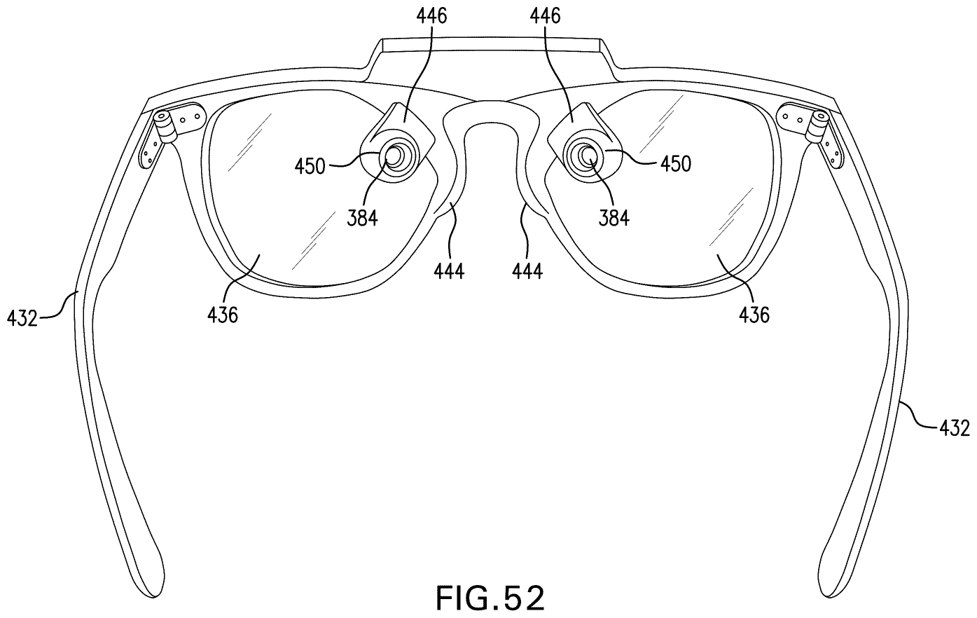

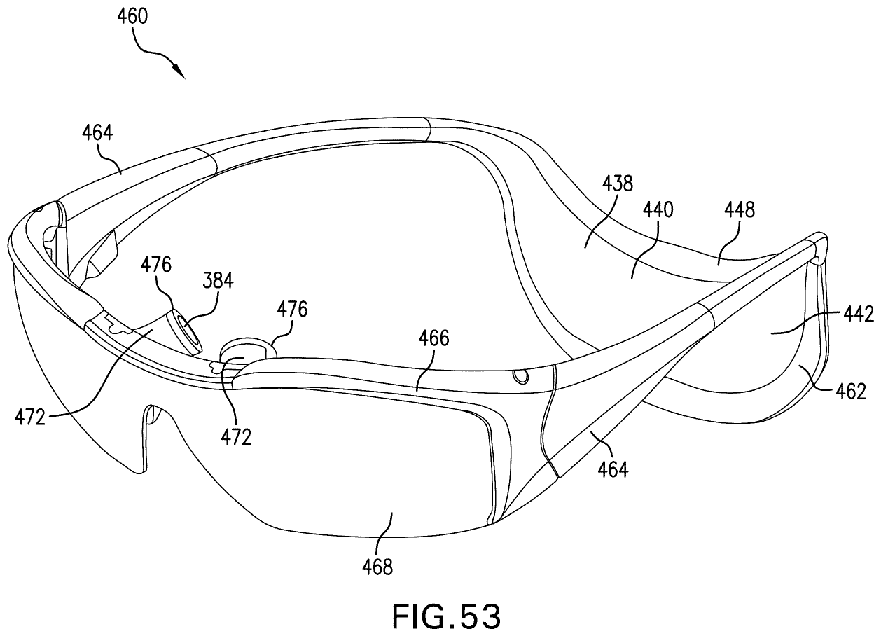

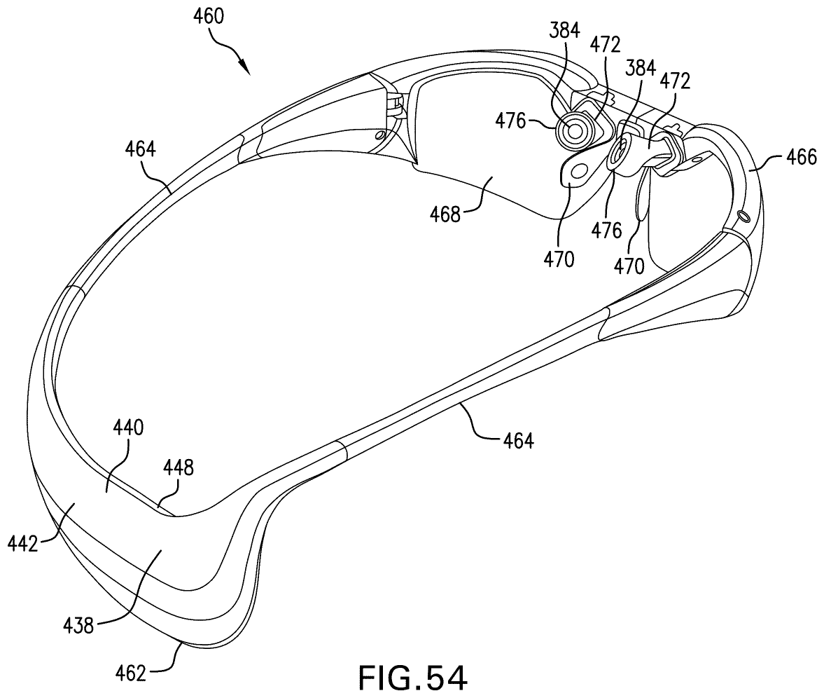

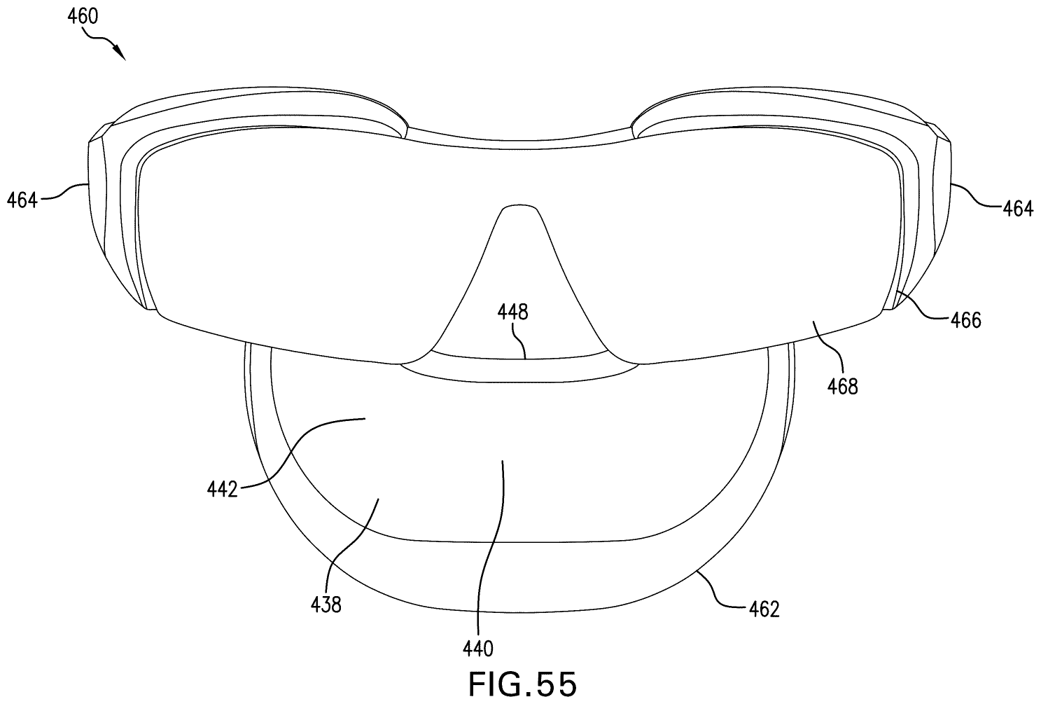

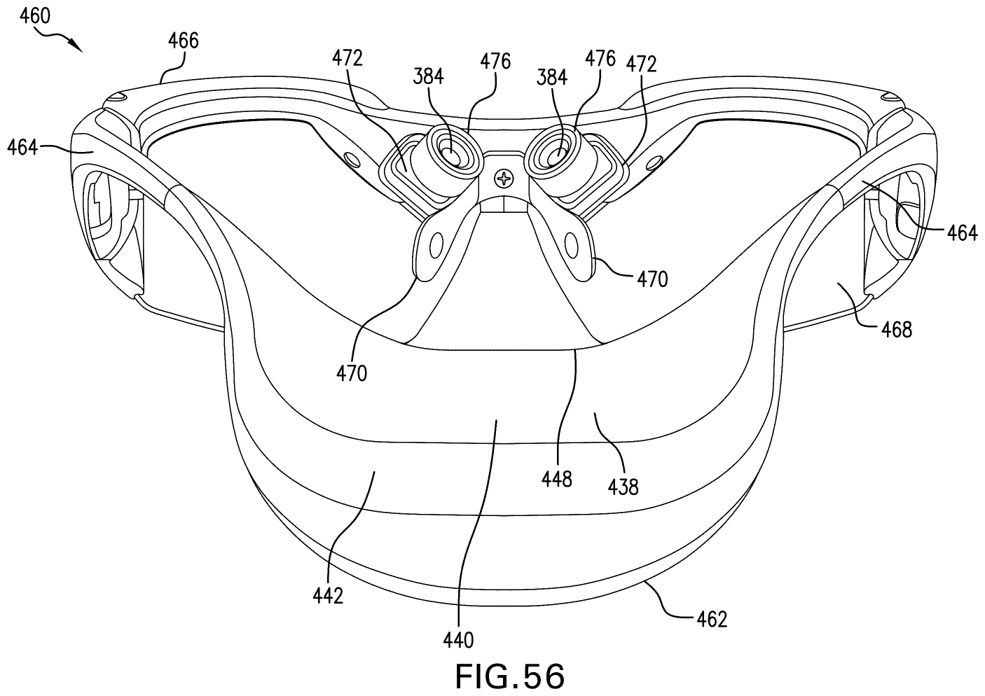

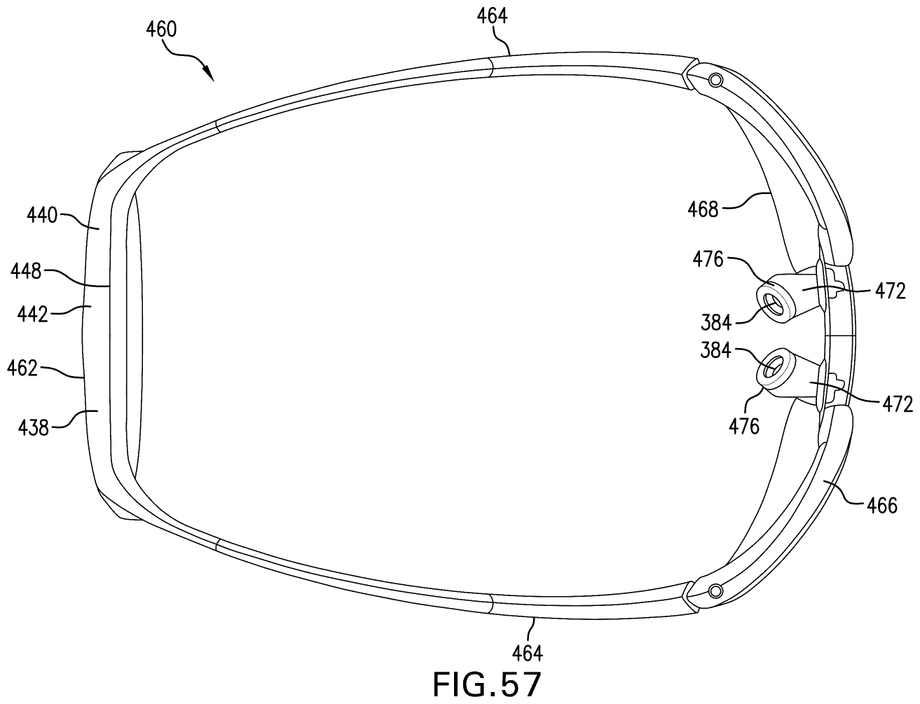

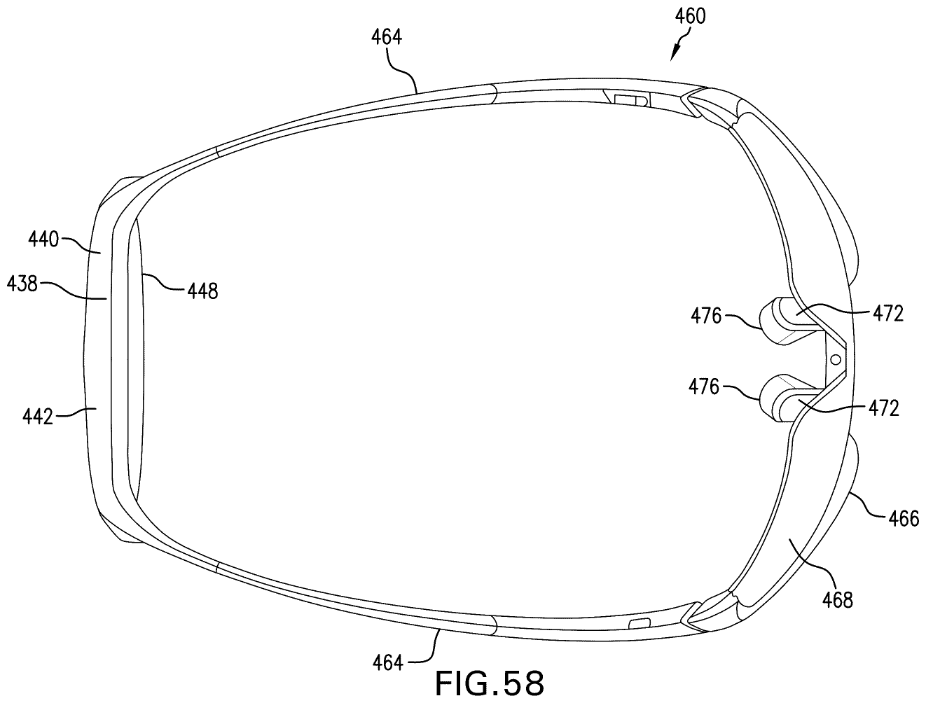

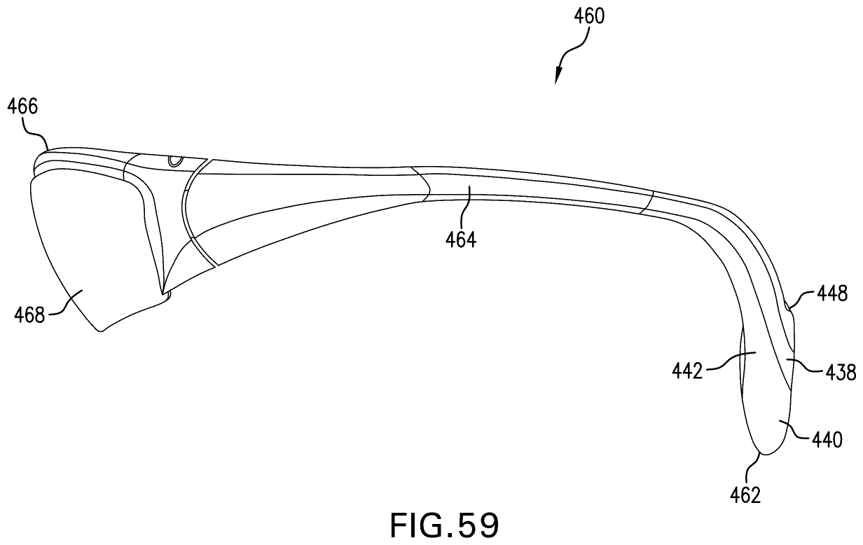

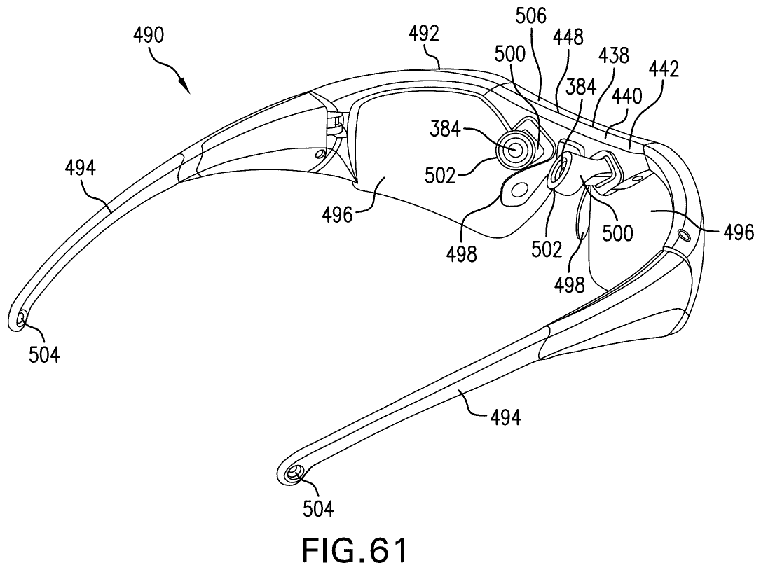

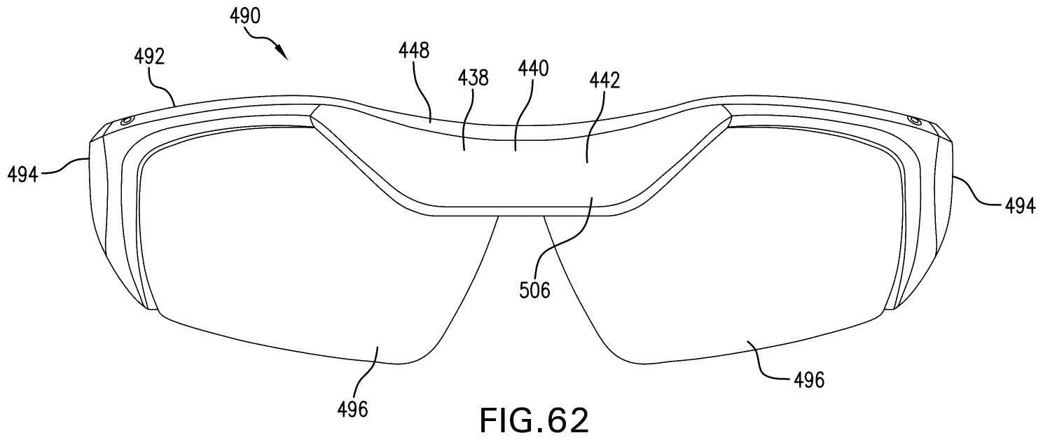

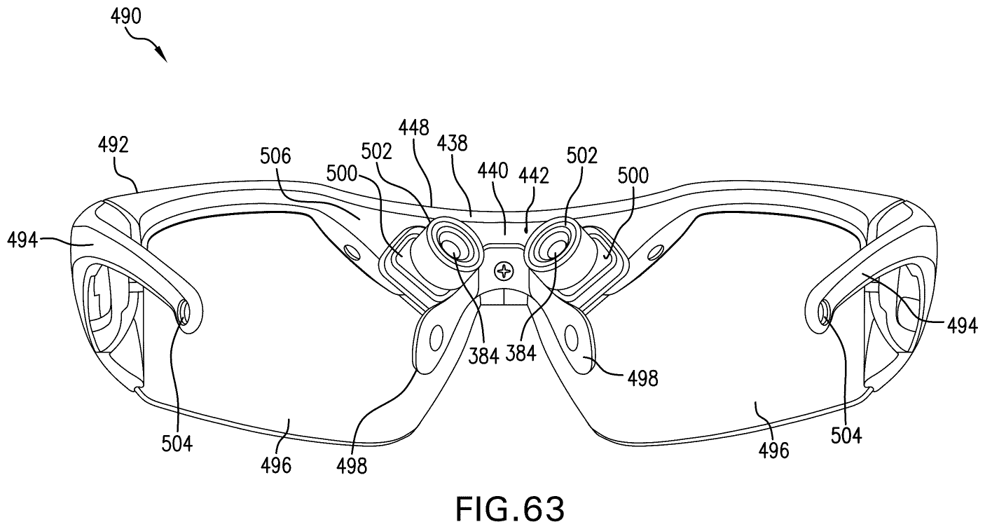

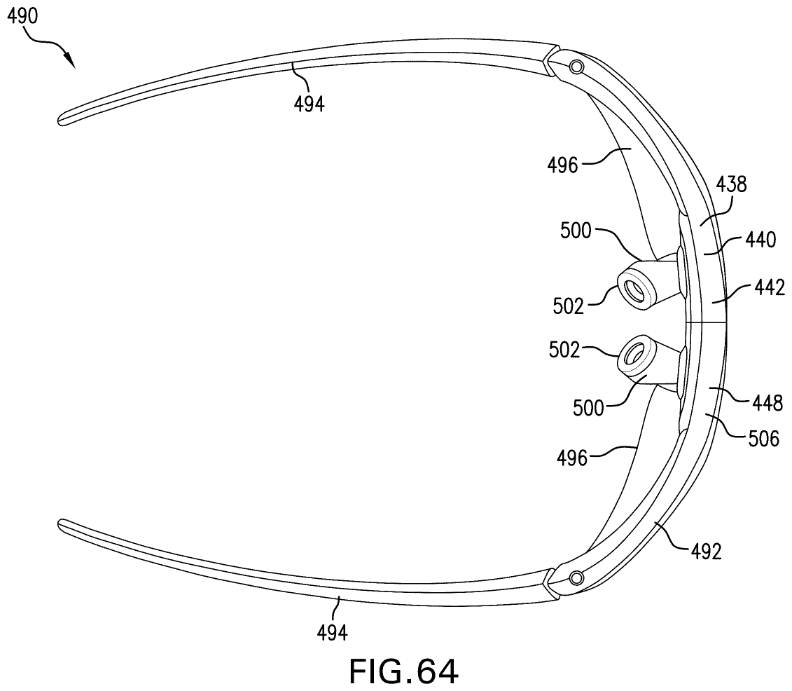

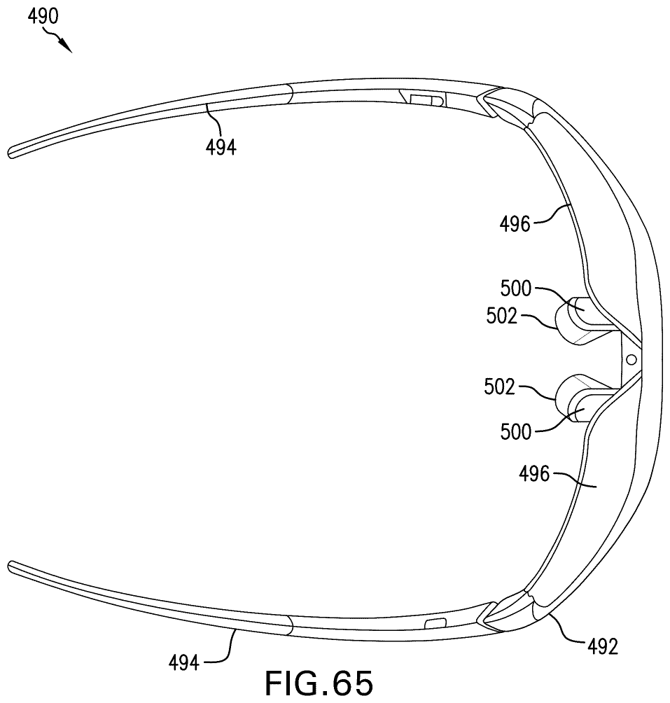

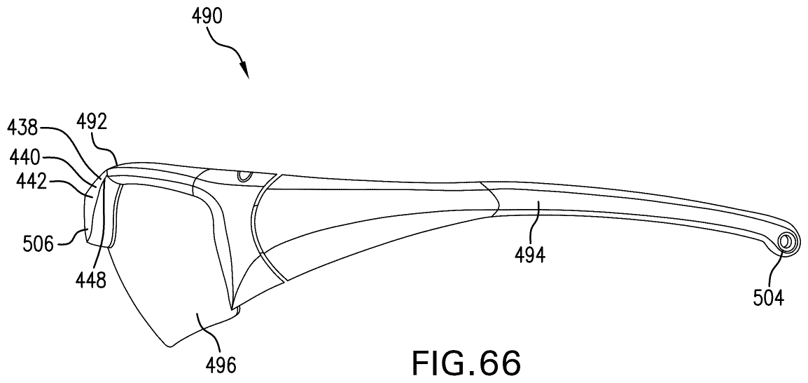

FIGS. 48-66 show embodiments of inductors positioned on eyeglasses. Except where such embodiments show different features, the reference numbers for similar elements are the same throughout all embodiments.

Eyeglasses 430 include frames that include temples 432 and eyeglass frame 434. Eyeglass frame 434 supports lenses 436. Supported internally to eyeglasses 430 is a power supply 438, a processor 440, and a transmitter 442 for communicating with the devices described elsewhere herein. Power supply 438, processor 440, and transmitter 442 can be positioned in temples 432 or in eyeglass frame 434 or split between temples 432 and eyeglass frame 434. Eyeglass frame 434 includes a nose piece 444 adjacent to each lens 436 for supporting eyeglasses 430 on a nose of patient 82. Eyeglass frame 434 also includes an inductor support 446 positioned between each nose piece 444 and a top of eyeglass frame 434. Each inductor support 446 includes inductor 384 oriented at an angle from the horizontal that enables inductor 384 to be positioned on a corresponding ABTT 90 of patient 82. The angle from horizontal is approximately 45 degrees. However, the angle from horizontal is preferably in the range from 10 degrees to 80 degrees, is more in the range of 15 degrees to 75 degrees, is even more preferably in the range of 30 degrees to 60 degrees, and is most preferably in the range of 40 degrees to 50 degrees. The optimal 45-degree angle allows inductor 384 to be aligned with ABTT 90 when eyeglasses 430 are worn by patient 82.

As described elsewhere herein, inductor 384 is a heat transfer device powered by power supply 438 and controlled by processor 440 by signals transmitted to transmitter 442 by a mobile communication device through an antenna 448. Each inductor 384 can be surrounded by an annular shield 450 that helps retain heat on a corresponding ABTT 90. It should be noted that each inductor 384 is positioned a spaced distance from eyeglass frame 434, which is necessary to reach a corresponding ABTT 90.

Eyeglasses 460 of FIGS. 53-59 are like eyeglasses 430 but include a frame 462 that can be used to house electronic devices of eyeglasses 430. Eyeglasses 460 include frames that includes temples 464 and eyeglass frame 466. Eyeglass frame 466 supports a single lens 468. Attached to a distal or back end of temples 464 is an additional frame 462 that serves as an electronics housing. Supported internally to additional frame 462 is a power supply 438, a processor 440, and a transmitter 442 for communicating with the devices described elsewhere herein. Additional frame 462 is relatively large, providing a larger volume for electronic components, particularly for power supply 438. Inductors 384 positioned on eyeglasses 460 require significant power, and additional frame 462 provides a relatively large volume for a power supply, which can include batteries, miniature fuel cells, and the like.

Eyeglass frame 466 includes a nose piece 470 on eyeglass frame 466 on each side of a gap formed in lens 468 for supporting eyeglasses 430 on a nose of patient 82. Eyeglass frame 466 also includes an inductor support 472 positioned between each nose piece 470 and a top of eyeglass frame 466. Each inductor support 472 includes inductor 384 oriented at an angle from the horizontal that enables inductor 384 to be positioned on a corresponding ABTT 90 of patient 82, as described herein. As described elsewhere herein, inductor 384 is a heat transfer device powered by power supply 438 and controlled by processor 440 by signals transmitted to transmitter 442 by a mobile communication device through antenna 448. Each inductor 384 can be surrounded by an annular shield 476 that helps retain heat on a corresponding ABTT 90. It should be noted that each inductor 384 is positioned a spaced distance from eyeglass frame 466, which is necessary to reach a corresponding ABTT 90.

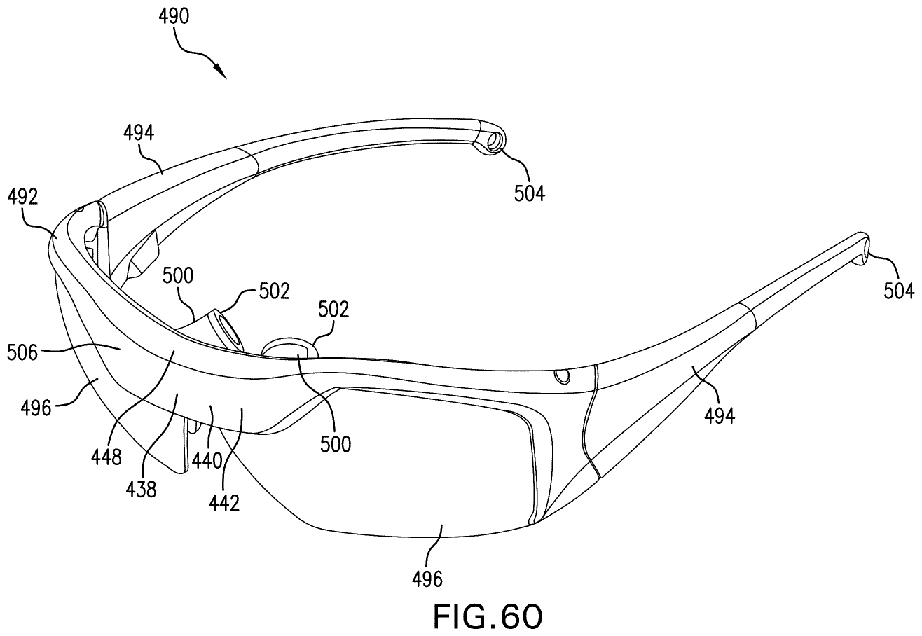

FIGS. 60-66 show eyeglasses 490 of FIGS. 53-59 in accordance with a further embodiment of the present disclosure. Eyeglasses 490 include frames that includes temples 494 and eyeglass frame 492. Eyeglass frame 492 supports a lens 496 on a right side of eyeglass frame 492 and on a left side of eyeglass frame 492. Eyeglass frame 492 includes a nose piece 498 adjacent to each lens 496 at a location adjacent to a gap formed between right lens 496 and left lens 496. Each nose piece 498 is configured to rest on the nose of patient 82 for supporting eyeglasses 490 on the nose of patient 82. Eyeglass frame 492 also includes an inductor support 500 positioned between each nose piece 498 and a top of eyeglass frame 492. Each inductor support 500 includes inductor 384 oriented at an angle from the horizontal that enables inductor 384 to be positioned on a corresponding ABTT 90 of patient 82. The angles from the horizontal are described elsewhere herein. As described elsewhere herein, inductor 384 is a heat transfer device powered by power supply 438 and controlled by processor 440 by signals transmitted to transmitter 442 by a mobile communication device through antenna 448. Processor 440, power supply 438, transmitter 442, and antenna 448 can be located in eyeglass frame 492 or temples 494. If positioned on eyeglass frame 492, processor 440, transmitter 442, power supply 438 and transmitter 442 can be located in an electronic chamber 506 integrally formed in eyeglass frame 492.

Each inductor 384 can be surrounded by an annular shield 502 that helps retain heat on a corresponding ABTT 90. It should be noted that each inductor 384 is positioned a spaced distance from eyeglass frame 492, which is necessary to reach a corresponding ABTT 90. Each temple 494 can also include a speaker 504 that is located near an ear of patient 82 when eyeglasses 490 are positioned on the head of patient 82.

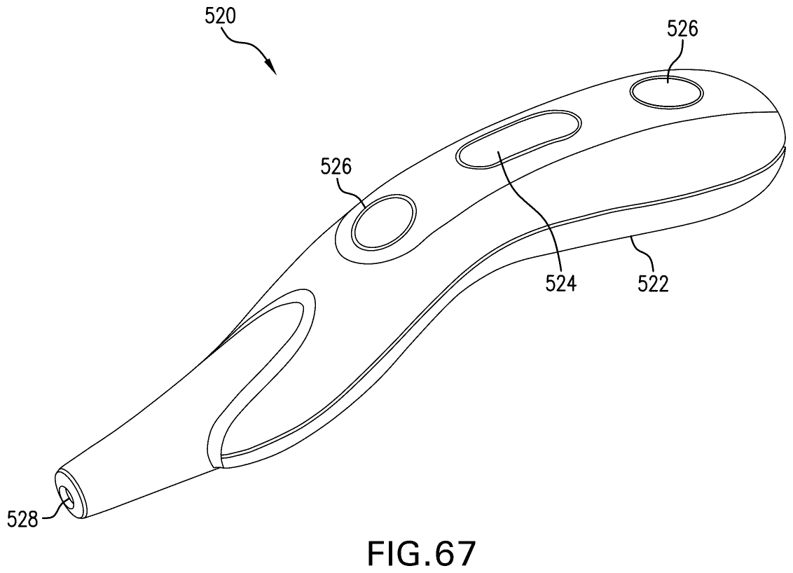

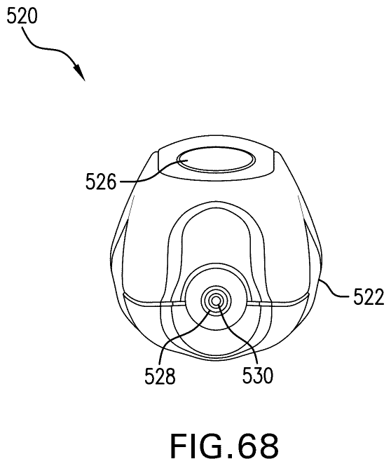

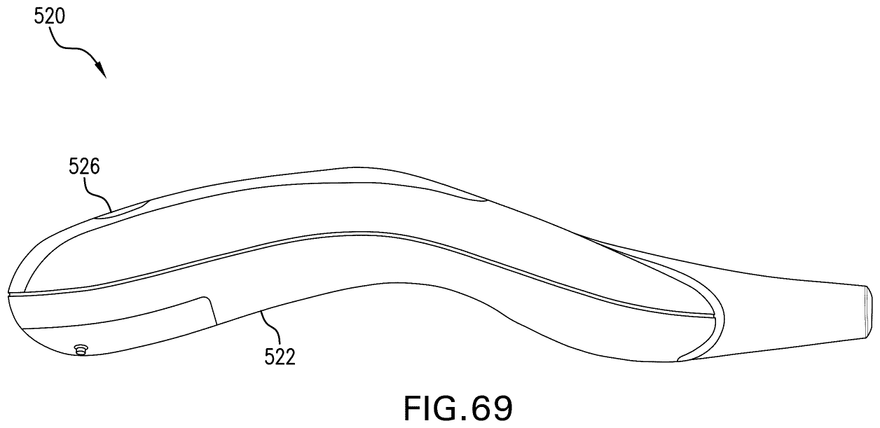

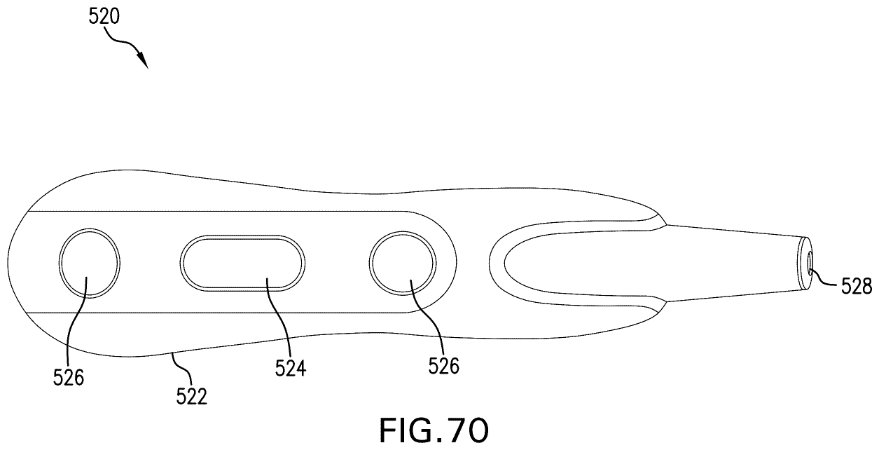

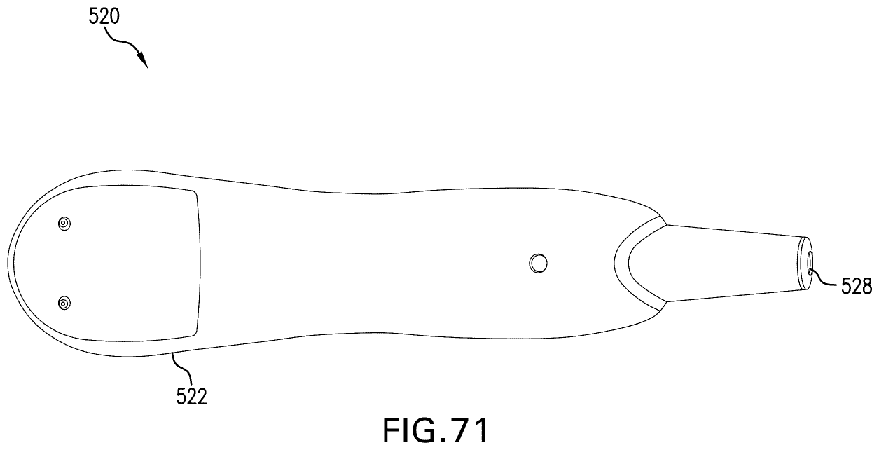

FIGS. 67 to 71 show a thermometer 520 in accordance with an exemplary embodiment of the present disclosure. Thermometer 520 includes a handle 522, a display 524, and controls 526 to turn thermometer 520 on and off, to start and stop temperature measurements, to change units of measure, and other control functions. Thermometer 520 includes an opening 528 at a distal end thereof inside of which is a temperature sensor 530 that can measure the temperature of a forehead or a single ABTT 90.

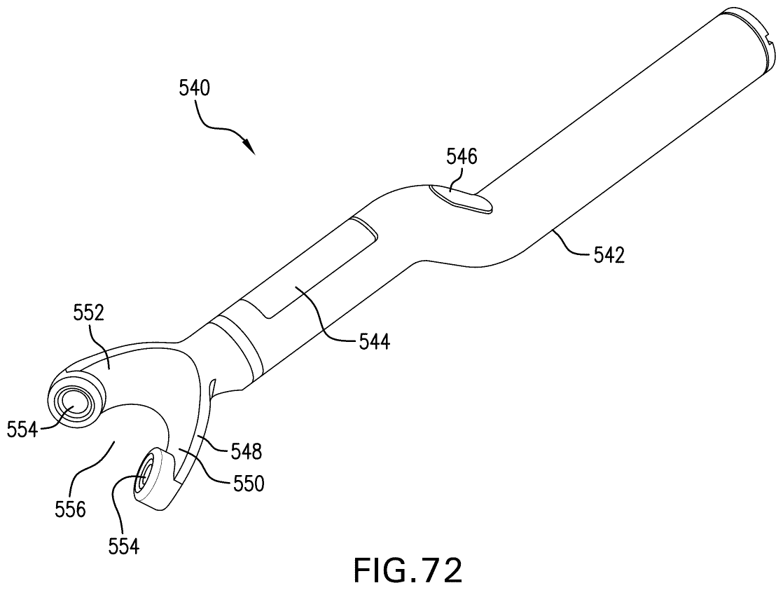

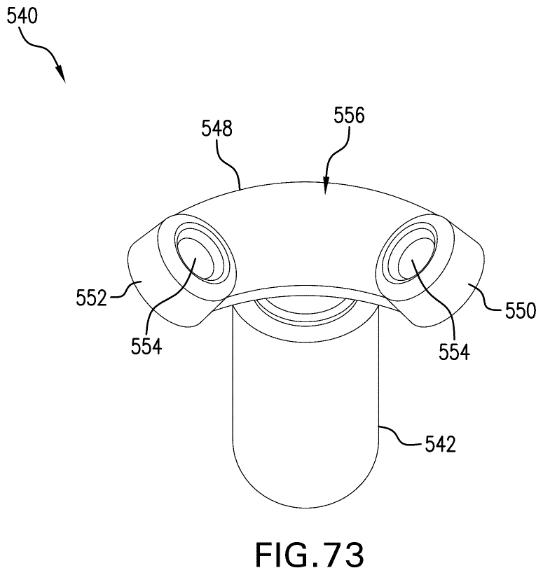

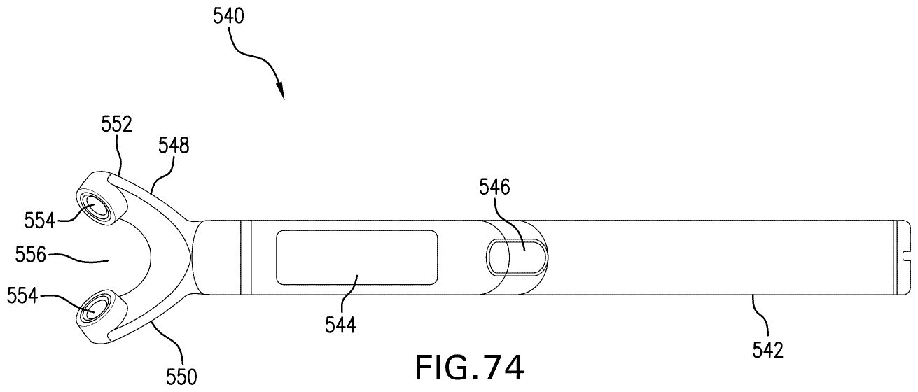

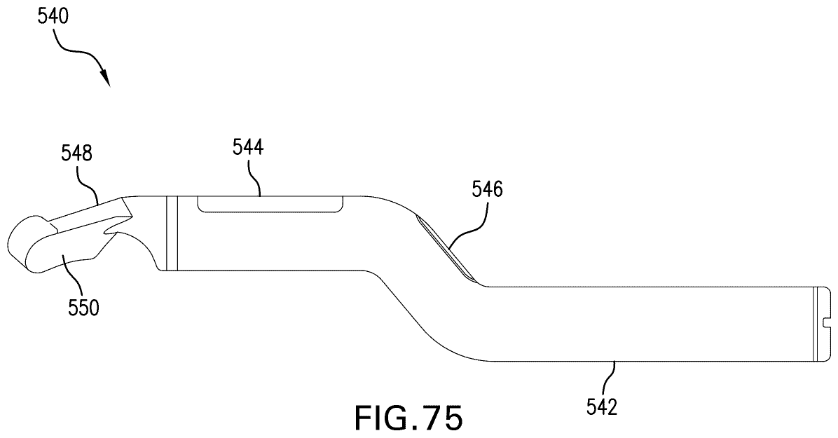

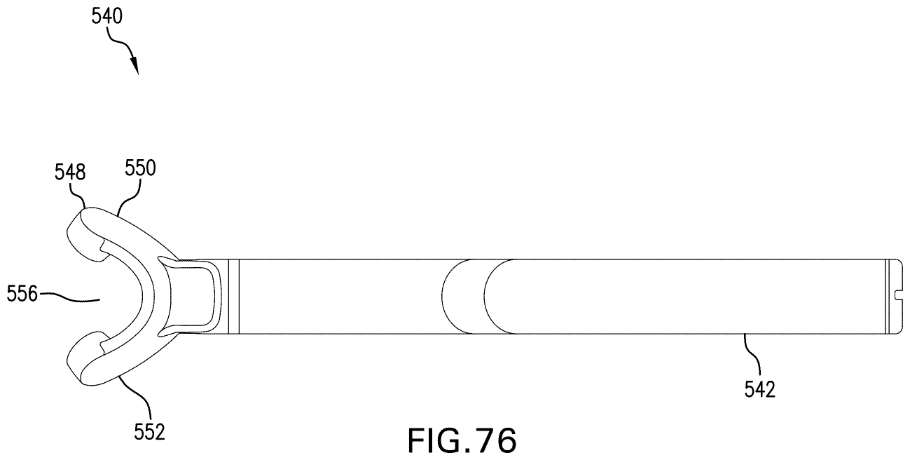

FIGS. 72 to 77 show a thermometer 540 in accordance with an exemplary embodiment of the present disclosure. Thermometer 540 includes a handle 542, a display 544, and at least one control 546 to turn thermometer 520 on and off, to start and stop temperature measurements, to change units of measure, and other control functions. Thermometer 540 includes a bifurcated temperature sensor support 548 which includes a first arm 550 and a second arm 552. Each of first arm 550 and second arm 552 supports a temperature sensor 554. Bifurcated temperature sensor support 548 includes a gap 556 to provide clearance for the nose of patient 82 so that temperature sensor 554 can be proximate to or in contact with ABTTs 90 with minimal and preferably no contact with the nose of patient 82.

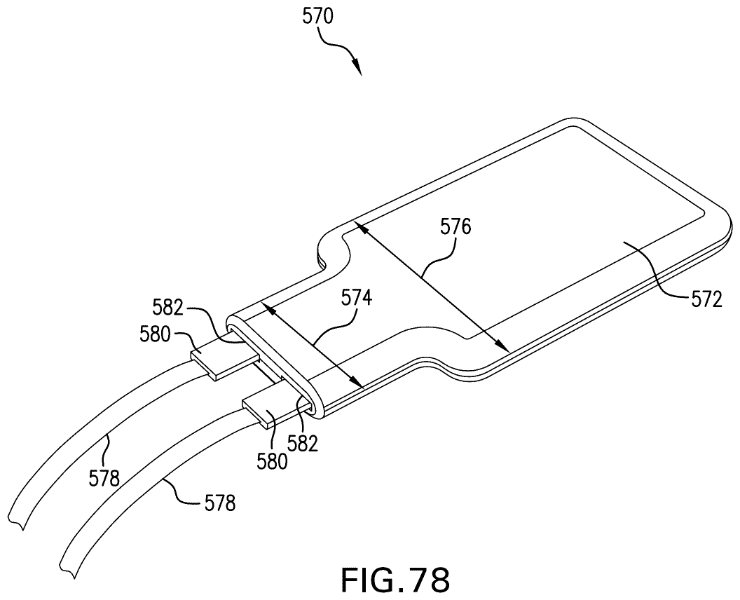

FIGS. 78-99 show views of various tongue heaters or inductors in accordance with exemplary embodiments of the present disclosure. The various embodiments all have one feature in common, a heated piece for insertion into a human mouth for heating of the human tongue. The various devices, which can be in a paddle configuration or a ball or bulb configuration, are generally fabricated from a highly conductive material, such as ceramic, copper, silver, or diamond-based materials. To avoid potential toxicity issues, inductors can be covered with a thin layer of food-grade plastic to prevent contact of the inductors with the saliva in the human mouth. Electrical leads from a power supply (not shown in FIGS. 78-99) can extend into the various heaters or inductors, though inductors may include internal fluid passages connectable to external fluid sources for providing heating and/or cooling. All electrical leads are insulated against voltage and current leakage, and a safety circuit can disconnect power if any electrical lead is disconnected from the inductor. The electrical leads can power a resistive heater or other suitable electrically powered heater.

Tongue heater 570 includes a flat paddle 572 with a width of paddle 572 increasing from a first width 574 to a second width 576. Second width 576 assists patient 82 in retaining flat paddle 572 in the mouth. Electrical wires 578 terminate in connectors 580 that are inserted into mating connectors 582 to power a resistive heater 584. Resistive heater 584 is covered by protective layer 586 that, as noted above, can be a food-grade plastic. Protective layer 586 is also electrically non-conductive while being relatively thermally conductive.

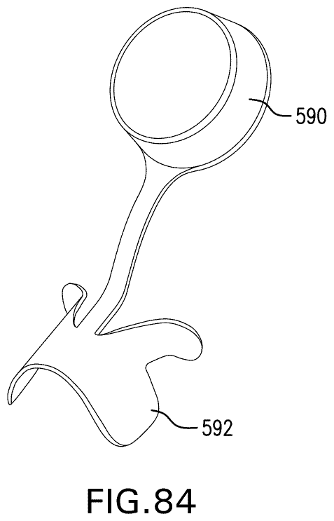

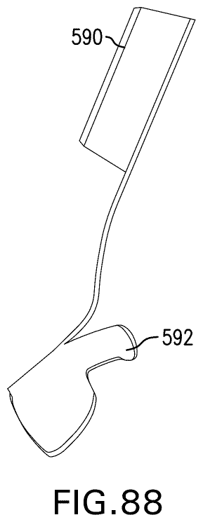

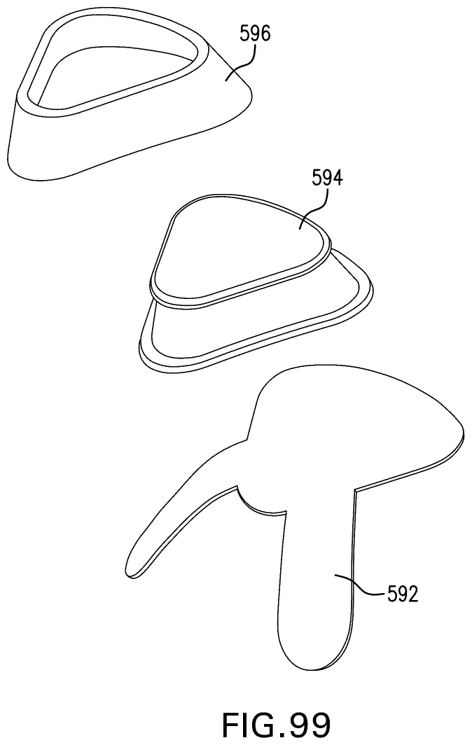

FIGS. 84-99 show tongue heaters with multiple nasal clip embodiments that help position and support the associated tongue heaters in the mouth of patient 82. The tongue heaters can be round, as shown in FIGS. 84-87, elongated, as shown in FIGS. 88-93, or triangular, as shown in FIGS. 94-99. In FIGS. 84-99, the tongue heaters are reference number 590, and the nasal clips are reference number 592. As shown in FIG. 99, tongue heater 590 can include a nasal clip 592, a heater 594, and an insulating ring 596 to minimize heat transfer to the cheeks and teeth of patient 82.

The primary heaters described to this point are full body heaters by way of a chamber, and tongue heaters. The most effective thermal cycles are those implemented by heat applied to the body of patient 82 in as many places as possible as quickly as possible. Accordingly, other types of heaters can be used to speed the delivery of heat for thermal cycles.

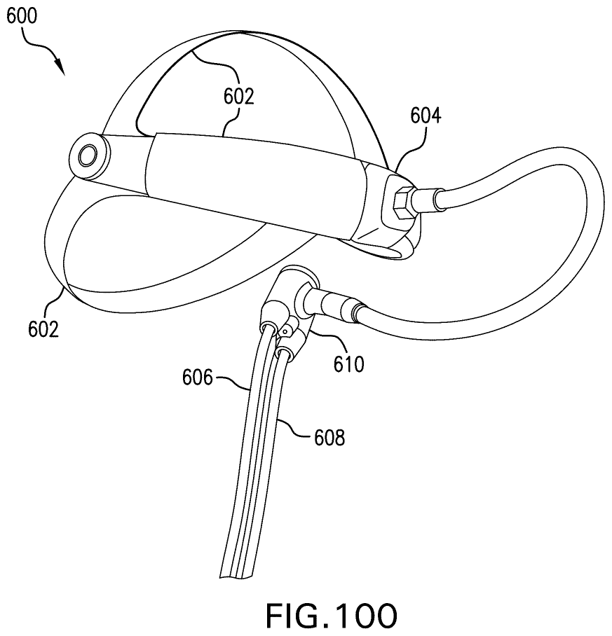

For example, FIG. 100 shows a face mask 600 with head straps 602 and a nasal interface 604. Heated air is supplied by nasal interface 604 by way of air hose 606 and air hose 608. Air hose 606 may provide cold air to patient 82 and air hose 608 may provide heated air to patient 82. Air hose 606 and air hose 608 terminate in a controllable air valve 610. By alternately controlling airflow from air hose 606 and air hose 608, patient 82 can be subject to rapid thermal cycles.

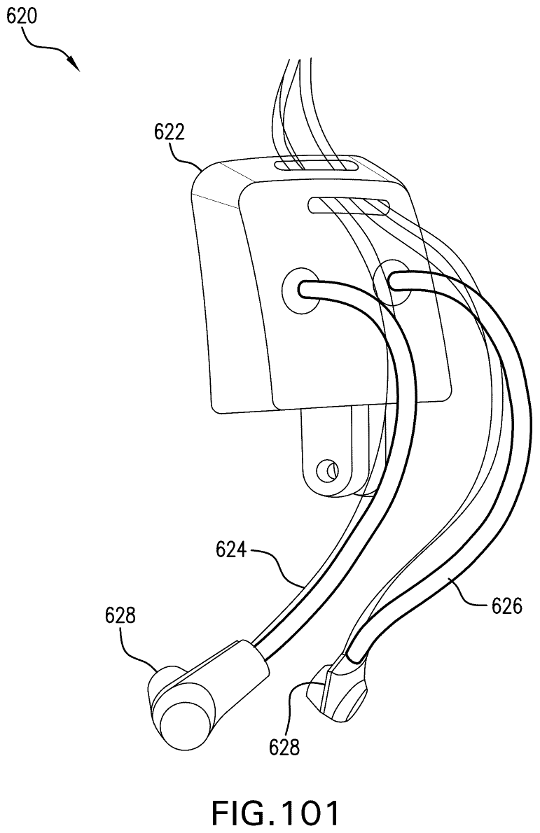

FIG. 101 shows an ABTT inductor assembly 620. ABTT inductor assembly 620 includes an inductor support 622, which can be attached to any of the head-supported devices disclosed herein. Inductor support 622 includes a first support arm 624 and a second support arm 626. Each of first support arm 624 and second support arm 626 includes an inductor 628 mounted on a distal end thereof. Each inductor 628 is movable by way of first support arm 624 and second support arm 626, each of which is flexible, to interface with a respective ABTT 90.

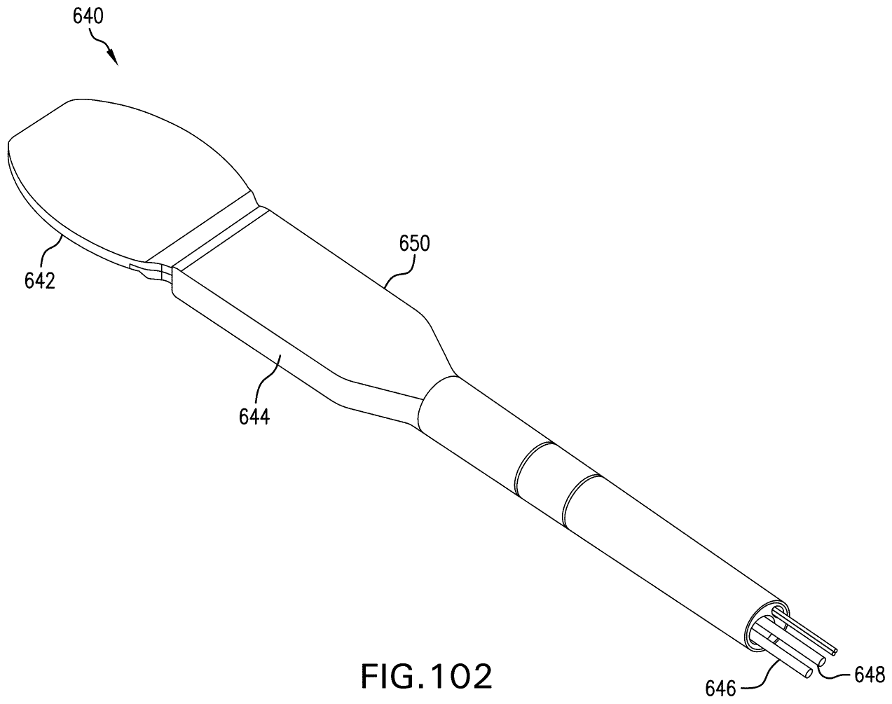

FIG. 102 shows another tongue heater 640. Tongue heater 640 includes a paddle 642, which can be made of copper or other thermally conductive material. Paddle 642 extends into heater 644, which can be a resistive heater, a thermoelectric heater, or other heater. Heater 644 is powered by a first power wire 646 and a second power wire 648. Heater 644 can be protected by a cover 650. Cover 650 can be replaceable and can be made of a food-grade or medical-grade plastic. As with other devices that extend into the mouth or nose of patient 82, paddle 642 can be covered by a thin protective layer to avoid direct contact of the material of paddle 642 with the salivary fluid in the mouth of patient 82.

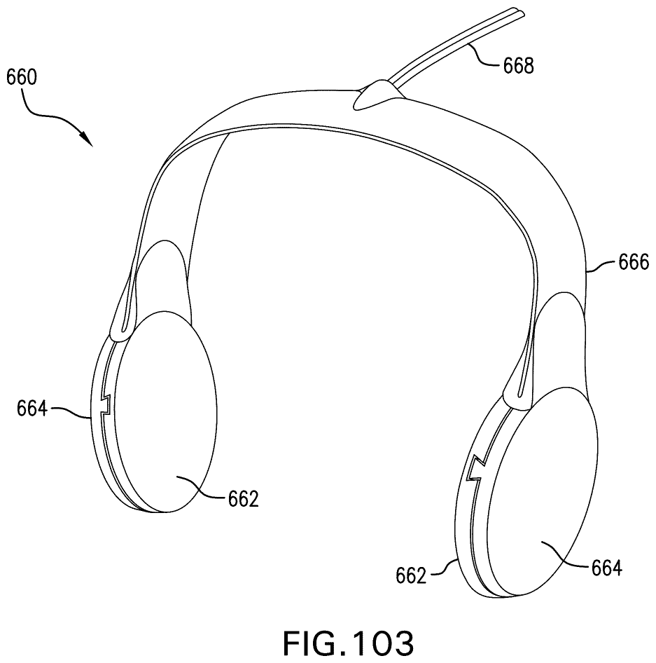

FIG. 103 shows a tympanum heater 660, which may be described as an ear heater. Tympanum heater 660 includes two ear paddles 662 for placing over the ears of patient 82. Each ear paddle 662 includes a heater 664 positioned directly or immediately adjacent to a back or outer side of two ear paddles 662. Tympanum heater 660 includes a headset 666 on which the components of tympanum heater 660 are mounted. Power for heater 664 comes from power wires 668.

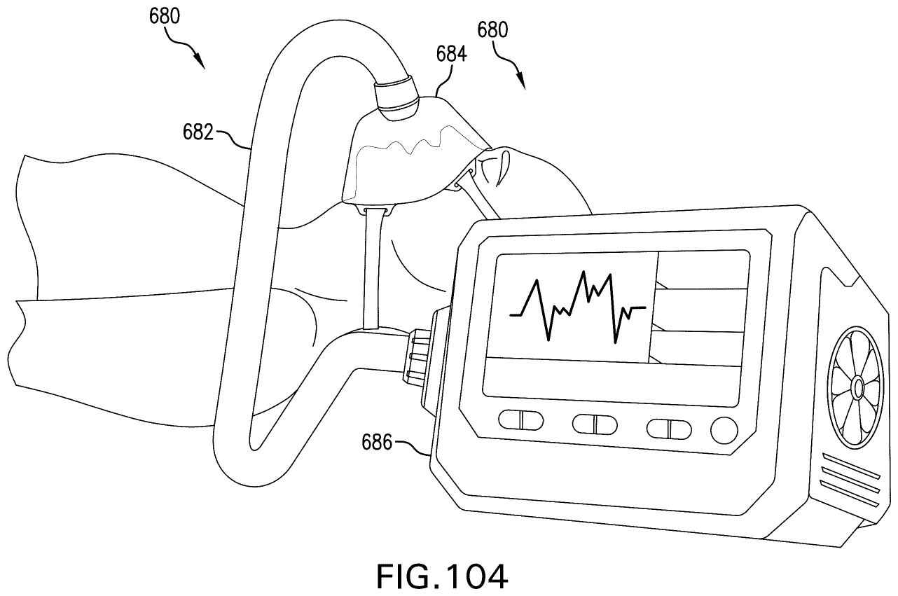

FIG. 104 shows an air-based oral heater 680, which may also be described as a lung inductor. Heated or cooled air is sent to patient 82 by way of an air hose 682 to a face mask 684 strapped onto patient 82. A blower unit 686 provides the heated or cooled air with an internal heater or cooler that may also include a humidifier for patient comfort.

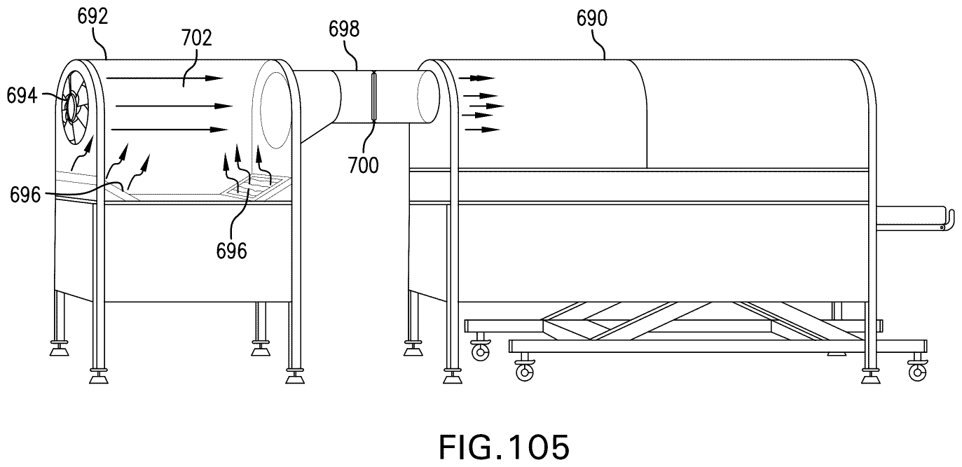

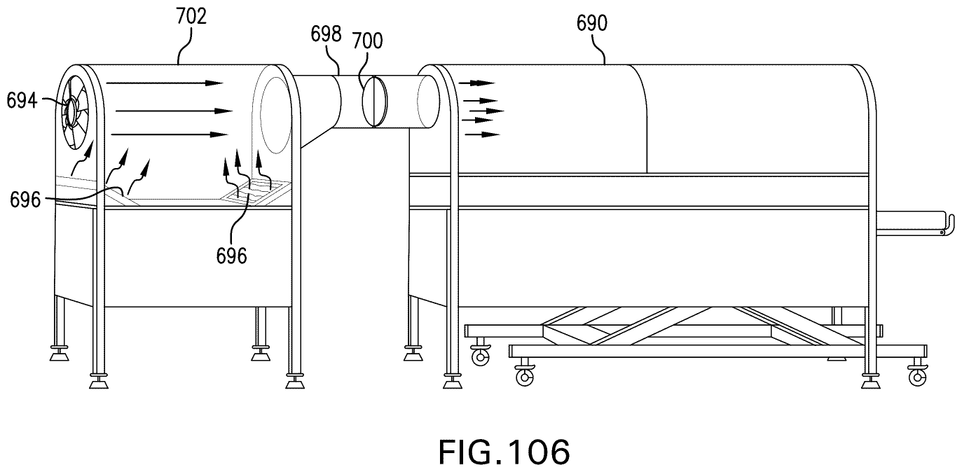

While many embodiments of chambers in the present disclosure include internal heaters, in another embodiment shown in FIGS. 105 and 106, a chamber 690, or any of the other chamber embodiments disclosed herein, can be connected to a pre-heat unit 692 by way of an air transport tube 698. Pre-heat unit 692 includes one or more heaters 696 positioned internally to a pre-heat chamber 702. A blower 694 is positioned in a wall of pre-heat chamber 702 on an opposite side of pre-heat chamber 702 from air transport tube 698. Air transport tube 698 can include a valve 700 to prevent hot air from readily flowing from pre-heat chamber 702 into chamber 690. During operation, one or more heaters 696 are turned on to heat the air in pre-heat chamber 702. When heat is needed in chamber 690, valve 700 in air transport tube 698 is opened, as shown in FIG. 106, and blower 694 is turned on to rapidly blow hot air into chamber 690 for fast heating of patient 82 positioned in chamber 690. While not shown, an additional chamber can be attached to chamber 690 that can provide pre-cooled air to 690 to provide fast thermal cycles for patient 82 by alternating between the hot air from pre-heat chamber 702 and the cold chamber. It should be noted that pre-heat unit 692 can be attached to interior 30 of any chamber disclosed herein to accelerate heating within interior 30.

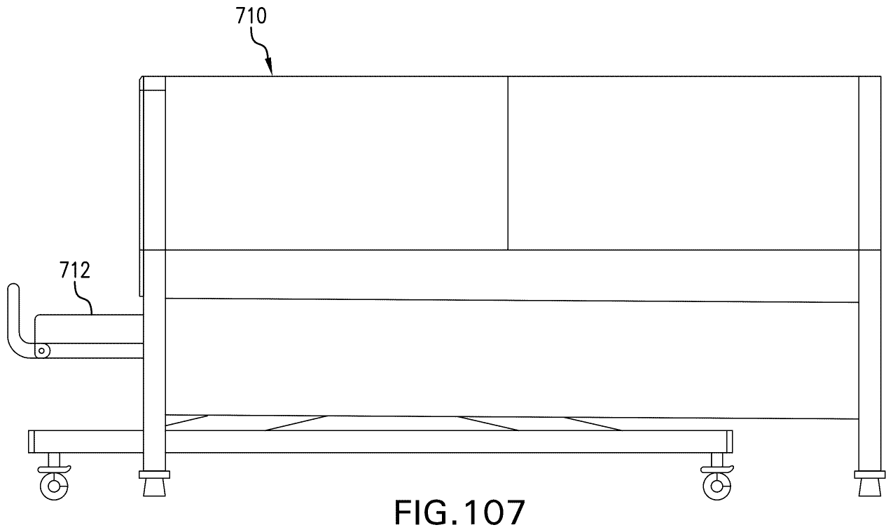

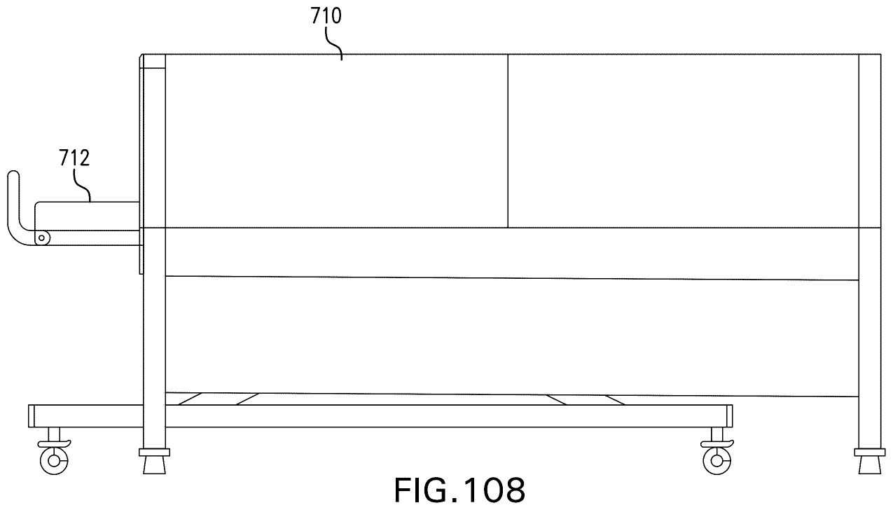

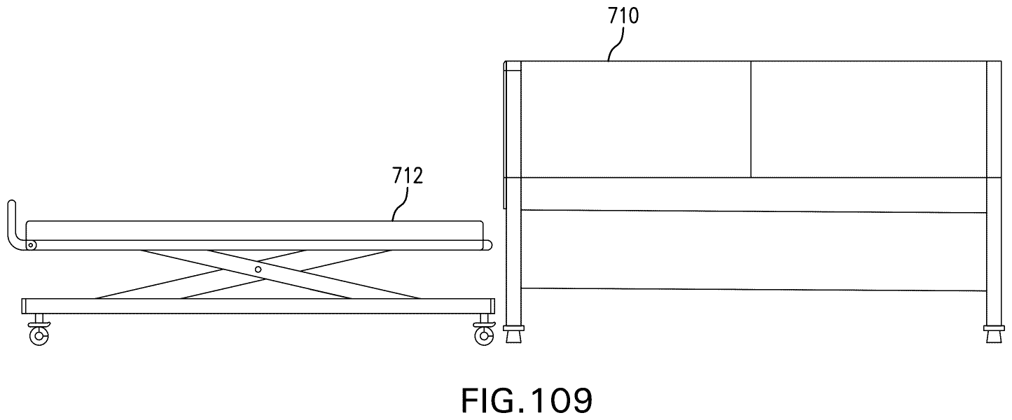

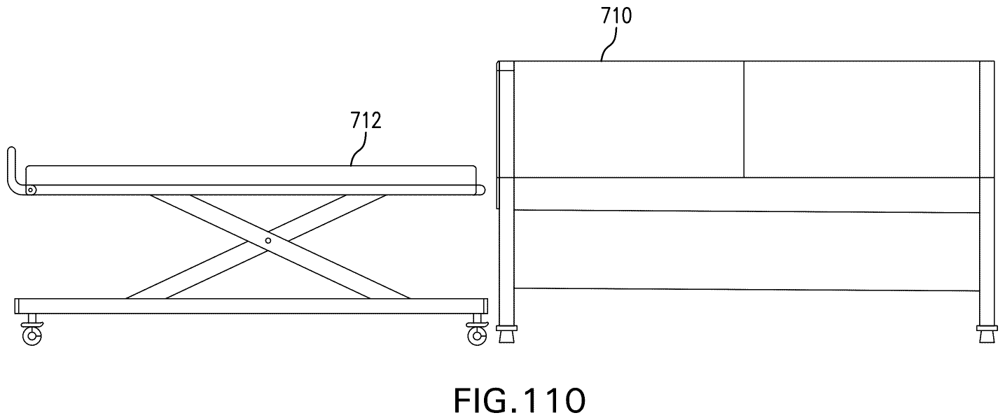

FIGS. 107 to 110 show a chamber 710 and a patient support 712 with patient support 712 in raised and lowered positions internal to chamber 710 and external to chamber 710. In the embodiment of FIGS. 107 to 110, which is similar to other embodiments including a chamber having interior 30 disclosed herein, patient 82 is placed on patient support 712. Patient support 712 is then lowered to go under the walls of chamber 710. Patient support 712 is then moved under chamber 710. Patient support 712 is then raised to place patient 82 in the heated zone of chamber 710. Patient support 712 in FIGS. 107 to 110 is in the form of a scissor-type lifting configuration that is compatible with any of the patient supports described herein.

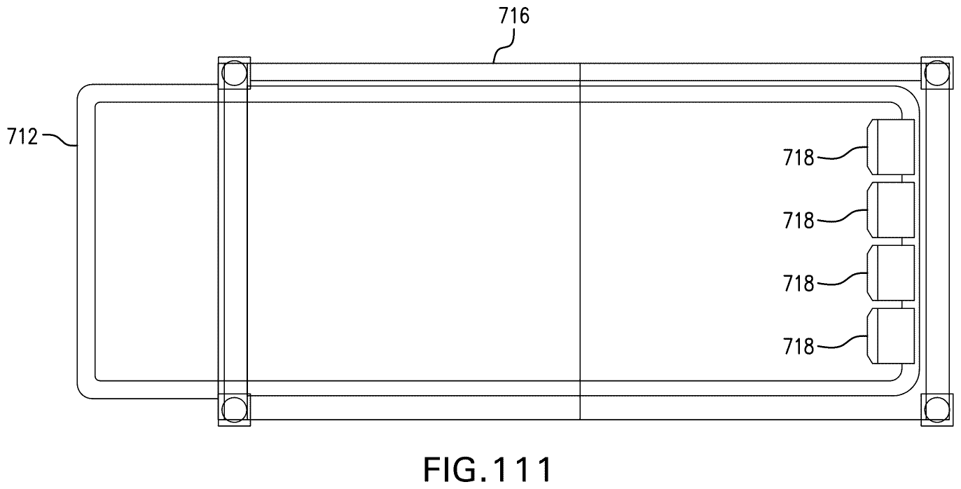

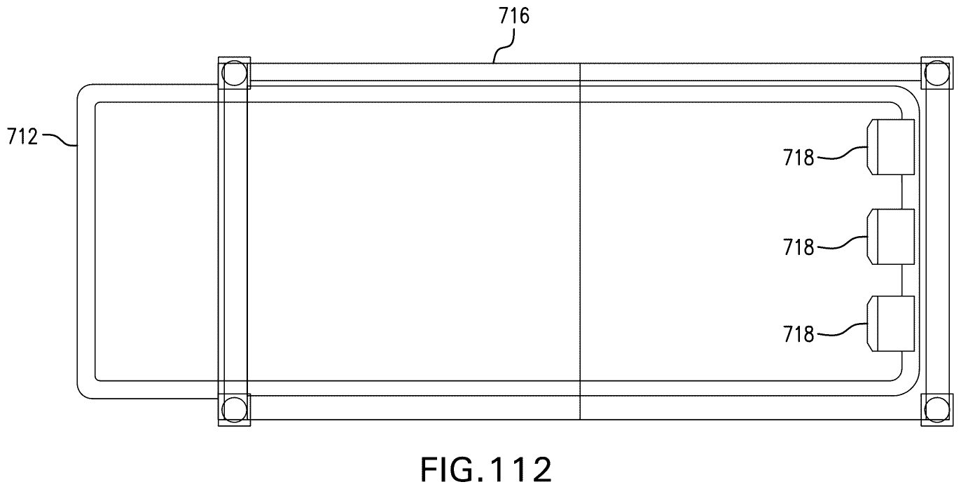

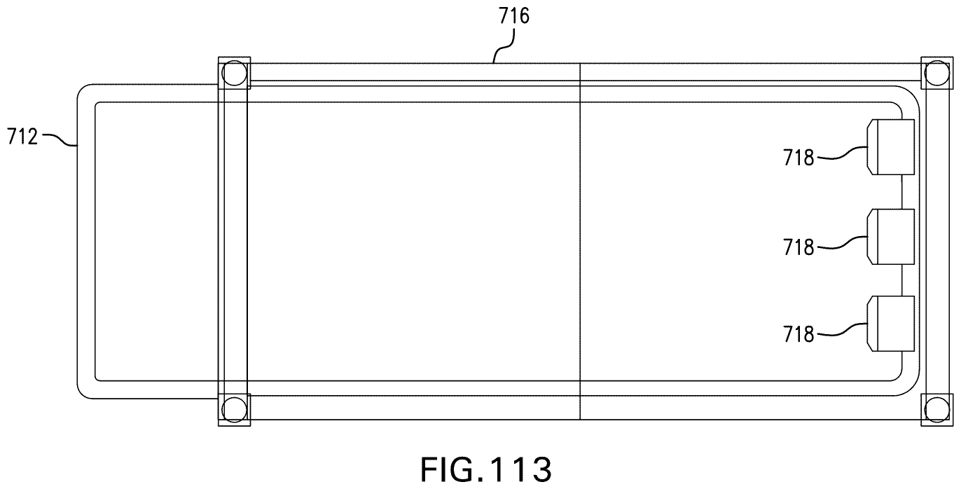

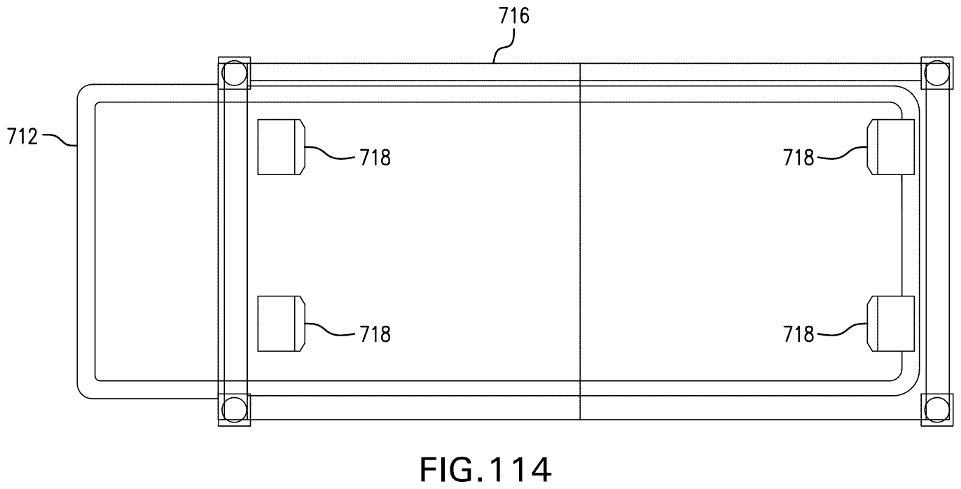

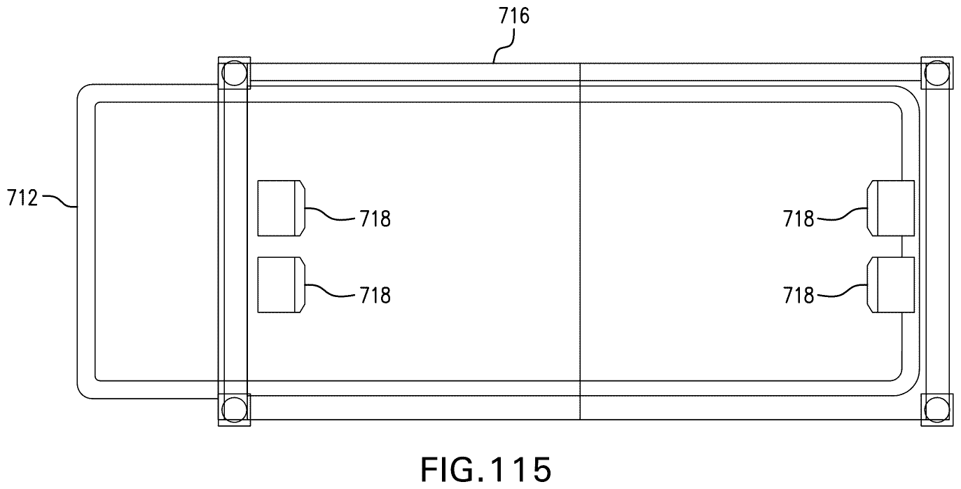

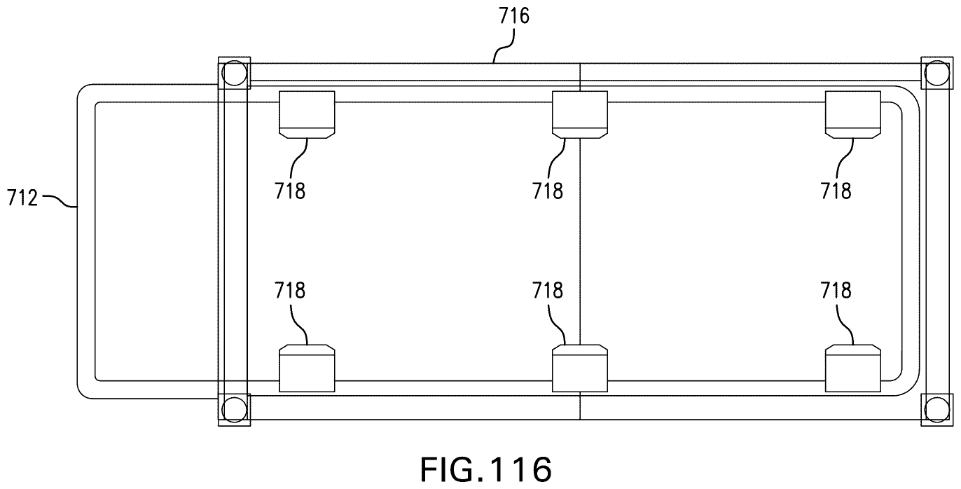

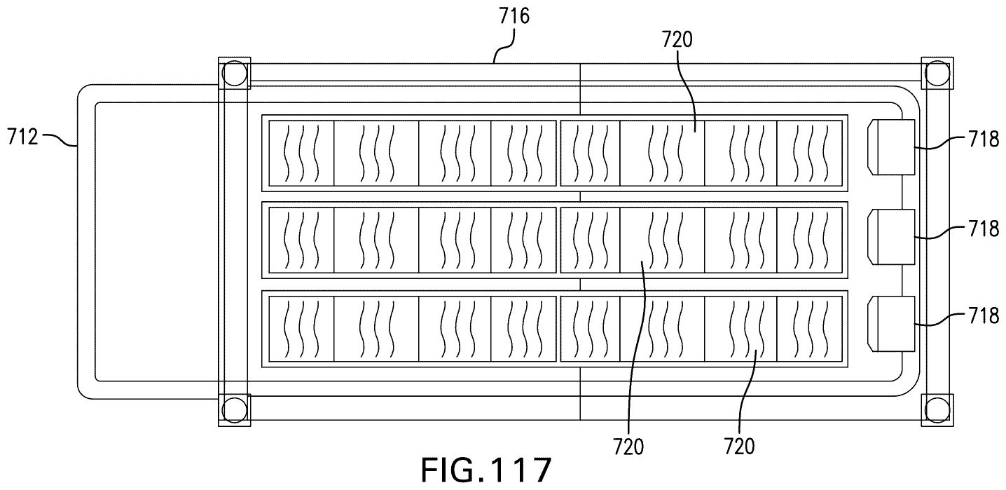

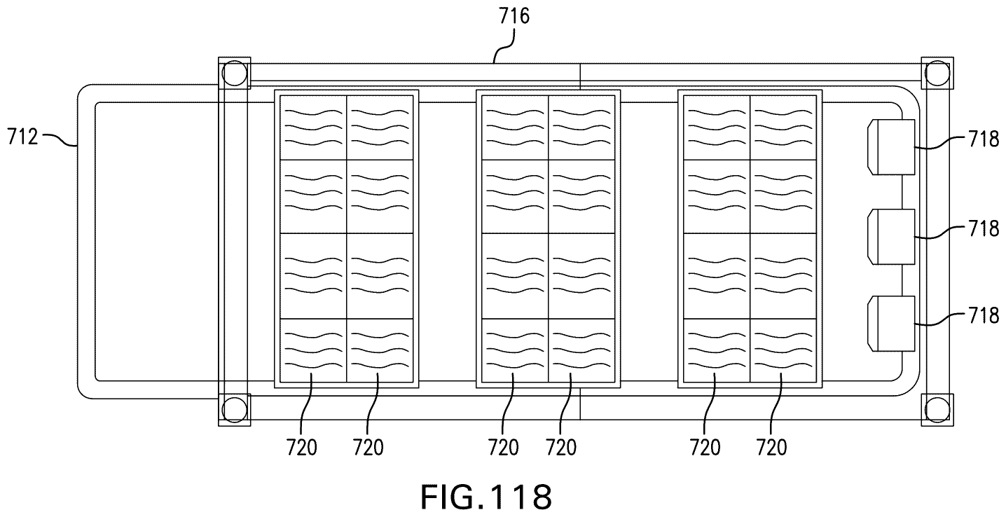

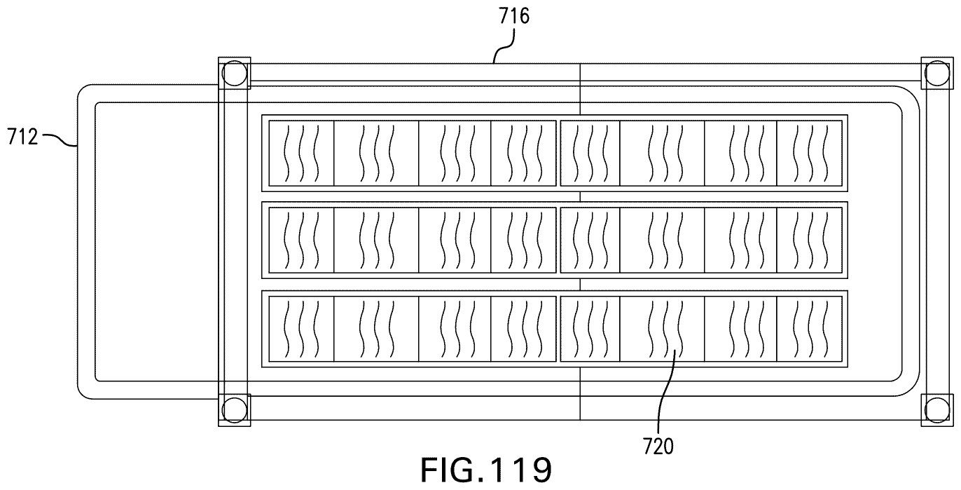

Many heater configurations are possible for the various chambers disclosed herein, and some configurations have already been shown. FIGS. 111 to 119 show other configurations. FIG. 111 shows chamber 716 with heaters 718 positioned along a back top interior of chamber 716 and the outputs of heaters 718 are longitudinal from a back or rear of chamber 716 to a front end or wall of chamber 716. While four heaters 718 are shown in FIG. 111, more heaters 718 or less heaters 718 can be used depending on the heat generation capability of heaters 718. FIGS. 112 and 113 show a configuration using three heaters 718, each of which outputs heat and/or heated air longitudinally from the back or rear of chamber 716 to the front of chamber 716. FIG. 114 shows a heater configuration where two heaters 718 are positioned at a top back or distal end of chamber 716 and two heaters 718 are positioned at a top front or proximal end of chamber 716. Heaters 718 at the top back send heat and/or heated air longitudinally toward the front of chamber 716 and heaters 718 at the top front of chamber 716 send heat and/or heated air longitudinally toward the back or rear of chamber 716. Heaters 718 in FIG. 114 are positioned closer to the sidewalls of chamber 716 than a longitudinal centerline of chamber 716. In FIG. 115, heaters 718 are positioned closer to the longitudinal centerline of chamber 716 than to the sidewalls of chamber 716. In FIG. 116, heaters 718 are spaced along a longitudinal length of chamber 716 nearer to the side of chamber 716 than to the longitudinal centerline of chamber 716. In the configuration of FIG. 116, heat and/or heated air is directed transversely to the longitudinal centerline of chamber 716. While heaters 718 may be blower type heaters, FIG. 117 shows radiating heaters 720 extending along the upper surfaces of chamber 716 along nearly an entire length of chamber 716 as well as heaters 718. This configuration enables extremely rapid heating of chamber 716, though requiring a more significant amount of power than some other embodiments. In the configuration of FIG. 118, heaters 720 extend from a first side of chamber 716 to a second, opposite side of chamber 716 in a plurality of locations. While multiple embodiments use a combination of heaters 718 and heaters 720, the configuration of FIG. 119 uses heaters 720 only. The primary difference between embodiments is the speed at which the various heaters can raise the temperature of chamber 716, which is a trade-off with cost since the more heaters, the more electricity is used to power the heaters.

In all embodiments with a plurality of heaters, heaters can be controlled individually to localize temperature application to specific parts of the body. The selection of heaters can be lateral or longitudinal to the extent that lateral and longitudinally arrayed heaters are present. Additionally, heaters can be angled to direct heat toward patient 82 or away from patient 82.

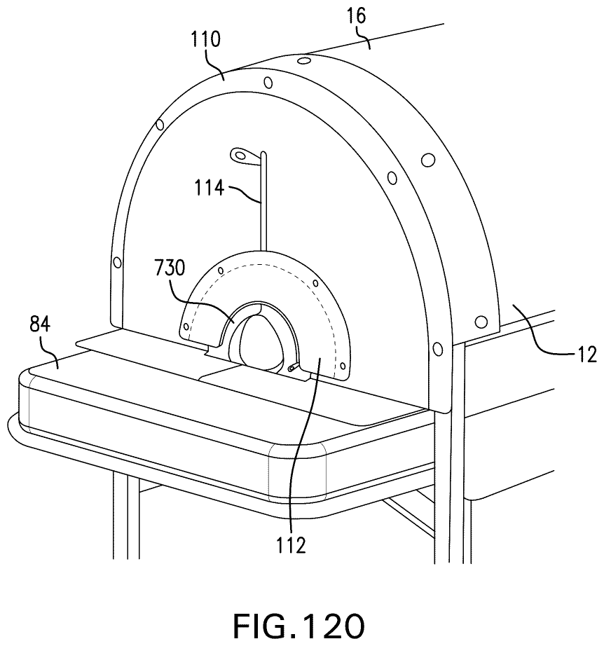

FIG. 120 shows a configuration similar to the configuration of FIG. 7 that includes a neck adapter 730 for filling the space between interior insulating layer 108, exterior cover layer 110, and the neck of patient 82.

FIG. 121 shows a simple chamber 734 with a flexible or compliant cover 736 at an opening to interior 30. A patient can readily be pushed through flexible or compliant cover 736 to interior 30, and flexible or compliant cover 736 can then be arranged in contact with the neck of patient 82 to contain heat in interior 30 of simple chamber 734.

The various embodiments herein are configured to provide heat cycles to patient 82. The heat cycles can be from 31 to 45 degrees Celsius in an exemplary embodiment. In a further embodiment, the temperature cycles can be 34 to 40 degrees Celsius. Because the temperature within a chamber is at a gradient, the maximum temperature in a chamber at the top of the chamber may be 120 degrees Celsius. However, patient 82 is positioned at a height level that corresponds with the desired temperature. Operation of the heaters can provide the specified temperature at the predetermined height of patient 82, or patient 82 can be raised or lowered into the proper temperature zone by raising or lowering the patient support or bed. The temperature cycles can be from 0.1 Hz to 120 Hz in an embodiment, though at higher frequencies the temperature cycles may have a lower magnitude. Such frequencies can be obtained in localized areas by way of high-velocity laminar flow air-based systems, though at relatively low amplitudes. In another embodiment, temperature cycles can be 0.5 Hz to 30 Hz. Again, as the frequencies move away from a few Hertz, the velocities can be obtainable by way of, for example, high-velocity laminar flow from an air-based system, though at relatively low amplitudes. Even so, ABTT 90 can respond to low-amplitude signals at high frequencies, recognizing the underlying frequency even though amplitudes can be as low as hundredths or even thousandths of a degree. As another example, increased frequency can be obtained in therapeutically effective locations, for example in the facial vein, the angular vein, the supraorbital vein, and the superior palpebral vein, by applications of a plurality of Peltier coolers, and operating the coolers at a time rate offset from each other. Thus, while the aggregate temperature of all Peltier devices may be in a band, the underlying frequency, which the hypothalamus can detect, can be one Hertz or more, limited only by the space available for the placement of Peltier cooler/heater devices.

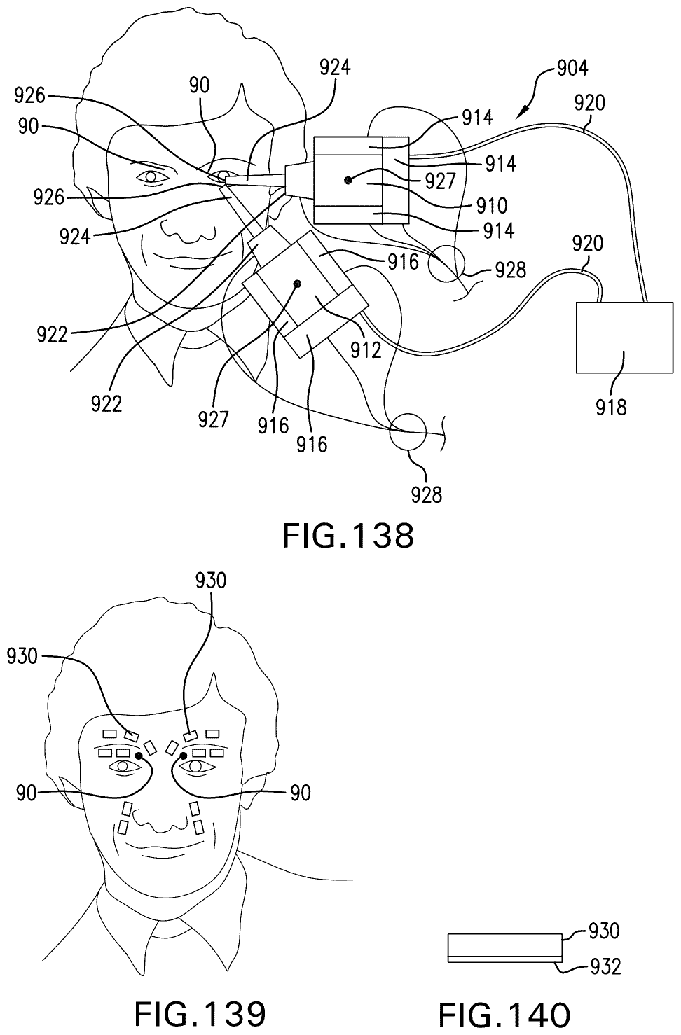

Turning to FIG. 138, a two-blower system 904 is shown. Two-blower system 904 includes a first blower 906 and a second blower 908. Each of first blower 906 and second blower 908 includes a reservoir 910 or a reservoir 912, respectively. Each reservoir 910 and 912 includes a plurality of heaters 914, a plurality of coolers 916, or combination heaters and coolers. Reservoir 910 and reservoir 912 are each sealed to hold pressure. The pressure can be, for example, up to 150 PSI. The pressure in reservoir 910 and reservoir 912 can be provided by, for example, an air compressor 918 by way of hoses 920. Attached to each reservoir 910 and reservoir 912 is a fast-acting valve 922 that is operable at frequencies up to at least 120 Hz. Extending from each 922/is an air nozzle 924 that has a narrow end 926 that enables close positioning of air nozzle 924 to ABTT 90. Because two narrow ends 926 need to be in proximity to ABTT 90, the diameter of each narrow end 926 in an embodiment is less than 0.50 inches, and is preferably less than 0.30 inches.

In operation, compressed air from air compressor 918 is provided to each reservoir 910 and reservoir 912. The plurality of heaters 914 is actuated to heat the compressed air in reservoir 910, and plurality of coolers 916 is actuated to cool the compressed air in reservoir 912. A computer, controller (control panel), and/or processor can be connected to each fast-acting valve 922, plurality of heaters 914, coolers 916, and air compressor 918 to control the operation of each device by way of a plurality of wires 928, or wirelessly, as described elsewhere herein. The processor, computer, controller, or control panel, which can be any of those devices described elsewhere herein, can be configured to set the predetermined temperature of the plurality of heaters 914 and plurality of coolers 916. A temperature sensor 927 can be provided on each reservoir 910 and reservoir 912 to feedback the temperature within reservoir 910 and reservoir 912. Each fast-acting valve 922 is operated to provide a specific frequency of temperature applied to ABTT 90. Because fast-acting valve 922 can be controlled at frequencies above 120 Hz, the frequency of temperature alternation applied to ABTT 90 can reach 120 Hz. Thus, for example, heated air at temperatures described elsewhere herein can be applied at, for example, a frequency of 60 Hz by way of fast-acting valve 922 positioned directly on reservoir 910. Similarly, cooled air at temperatures described elsewhere herein can also be applied at, for example, a frequency of 60 Hz by way of fast-acting valve 922 positioned directly on reservoir 912. However, the opening of fast-acting valve 922 on reservoir 910 will be offset from the opening of fast-acting valve 922 on reservoir 912 so that heated and cooled air is directed toward ABTT 90 in sequential pulses. Thus, ABTT 90 can be exposed to temperature cycles similar to those shown, for example, in FIGS. 122-125. Further, because the distance from reservoir 910 to narrow end 926 is relatively short, the temperature at the opening of narrow end 926 is close to the temperature of compressed air in reservoir 910. Thus, the temperature of compressed air emitted from narrow end 926 of air nozzle 924 is close to the temperature in reservoir 910. The same temperature relationship also holds for reservoir 912 and the cool air emitted or issued from narrow end 926 of air nozzle 924 connected to reservoir 912.

The same concept shown in FIG. 138 can be similarly applied to an entire body of patient 82. More specifically, a plurality of reservoirs 910 and reservoirs 912 can be positioned over an entire patient 82 so that heating of either all of patient 82 or specific parts of patient 82 can be accomplished by a plurality of reservoir 910 and reservoir 912, enabling high-frequency temperature cycles, which in the context of this disclosure is up to 120 Hz, applied to a large area of patient 82, potentially nearly the entire body of patient 82 with the addition of enough reservoirs 910 and reservoirs 912.

Turning to FIGS. 139 and 140, another system for applying high frequency heating and cooling is shown. Here a plurality of thermoelectric devices 930 are positioned along one or more of the facial vein, the angular vein, the supraorbital vein, and the superior palpebral vein. Such plurality of thermoelectric devices 930 can be, for example, a high-speed thermoelectric device from Kyocera that responds at more than 8 degrees Celsius per second, thus potentially enabling temperature cycles between 30 Hz and 60 Hz. Further, by alternating between the plurality of thermoelectric devices 930, for example, by heating with half of the plurality of thermoelectric devices 930 and then immediately cooling with the other half of the plurality of thermoelectric devices 930, temperature cycles induced into one or more of the facial vein, the angular vein, the supraorbital vein, and the superior palpebral vein, and potentially ABTT 90, can exceed 60 Hz by permitting the cooling of heated thermoelectric devices 930 while immediately cooling the previously un-actuated thermoelectric devices 930 of the plurality of thermoelectric devices 930. The same concept can be applied to the body of patient 82 by placing the plurality of thermoelectric devices 930 over much or all of the body of patient 82.

Referring to FIG. 140, each of the plurality of thermoelectric devices 930 can replace a conductive layer 932 that is positionable on the face of patient 82 or the body of patient 82 with a diamond-based material. Diamond conductors have conductivity up to five times as high as copper. Thus, the thermal efficiency of the plurality of thermoelectric devices 930 can be increased by replacing the contact surface with a diamond-based material, enabling a higher frequency of operation for the therapeutic application of thermal therapy. It should be understood that the therapeutic application of thermal therapy includes the application of at least one of heat cycles and cooling cycles, but can include the application of both heat cycles and cooling cycles in an alternating configuration, such as is shown in, for example, 122-125.

It should be noted that different frequencies can be applied at different amplitudes to different parts of the body, for example, specifically to the area near specific organs, for example, the heart, the liver, and the head, a large area of the torso, and/or to the extremities. Thus, while ABTT 90 may receive one frequency and amplitude of thermal cycles about a baseline, other portions of the body, such as head 83, and specific portions of the torso corresponding to the aforementioned organs, can receive a different amplitude, frequency, and/or temperature in therapeutically effective combinations.

Temperature cycles are matched to various brain functions to maximize the generation of heat shock proteins. 40 Hz to 100 Hz is optimal for gamma brain waves. 12 Hz to 40 Hz is optimal for beta brain waves. 8 Hz to 12 Hz is preferred for alpha brain waves. 4 Hz to 8 Hz is preferably matched with theta brain waves. Greater than 0 Hz to a maximum of 4 Hz is matched with delta brain waves. An alternative embodiment for matching with delta brain waves is 0.5 Hz to 4 Hz. Greater than 0 Hz to a maximum of 0.1 Hz can be matched with infra-low brain waves. An alternative frequency range is 0.65 Hz to 3.3 Hz, which is a match with heart functions. Another alternative frequency range is 0.1 Hz to 1 Hz, which is associated with respiratory functions. Further frequency ranges can be 4 Hz to 40 Hz and 8 Hz to 40 Hz to treat multiple brain wave frequencies.

It should be apparent that frequency cycles can target more than one issue with patient 82 at a time. For example, temperature cycles at a frequency of 0.65 Hz to 1 Hz will affect heart function, respiratory function, and brain delta wave function. Accordingly, multiple issues can be addressed simultaneously. The frequency ranges that can address multiple functions can be:
(A) 0.1 Hz to 100 Hz
(B) 0.1 Hz to 40 Hz
(C) 0.5 Hz to 40 Hz
(D) 0.5 Hz to 30 Hz
(E) 1.0 Hz to 30 Hz
(F) 0.6 Hz to 12 Hz

The time of application of the thermal cycles is dependent on the exact condition being treated. Application times can be 10 seconds to 5 hours, though are more typically 10 seconds to 2 hours. Also, while frequencies can be fixed for short intervals, frequencies can vary continuously with time. Indeed, it can be advantageous to vary thermal frequency continuously to prevent thermal fatigue, which can lead to a decrease in the benefits of thermal therapy. During the application of thermal cycles, hydration and patient temperature are monitored carefully to avoid pulmonary edema. Core temperature is also monitored at one or more locations, including tympanic, esophageal, rectal, and/or bladder. If any core temperature reaches a predetermined value, such as, for example, 42 degrees Celsius, the system can go into immediate emergency shutdown. All heating systems are turned off. An overtemperature warning can be set on any display or monitor, and an alarm can sound. If the patient support is motorized, the system removes patient 82 from the chamber immediately. Otherwise, attendant medical personnel remove patient 82 from the chamber immediately.

It should be noted that heat can be applied from multiple sources, as described herein. Those sources include chamber embodiments, heated air into the oral cavity and lungs, a conductive heater in contact with the tongue, and tympanic heaters in contact with the ears.

Other exemplary temperature ranges can be 34 degrees Celsius to 40 degrees Celsius centered on 37 degrees Celsius, and 35 degrees Celsius to 39 degrees Celsius centered on 37 degrees Celsius at the frequencies described above.

Another variable in the application of heat is the velocity or rate of change of heat applied to patient 82. A preferred temperature velocity or rate is 0.001 degrees Celsius per minute to 35 degrees Celsius per minute. A more preferred temperature velocity or rise rate is in a range from 0.005 degrees Celsius per minute to 35 degrees Celsius per minute. Other specific exemplary temperature rise rates can be 1.0 degrees Celsius per minute and 1.2 degrees Celsius per minute. Indeed, a preferred initial maximum temperature rise rate can be 1.5 degrees Celsius per minute, but 1.2 degrees Celsius per minute may be more preferred. The primary goal of the systems of the present disclosure is to generate heat shock proteins. However, there is a problem with consistently and continuously generating such proteins. While the thermal cycles disclosed herein generate such proteins, there is an accommodation effect where the temperature cycles no longer generate an effective level of heat shock proteins because the human body becomes accustomed to the temperature cycles. To sustain a therapeutically effective level of heat shock proteins, the system and method of the present disclosure overlays the temperature cycles described above on a longer temperature cycle, as can be seen in FIGS. 122-125.

In FIG. 122A, an upper curve shows how heat shock protein generation decreases with time. To combat such degradation, and as shown in FIG. 122B, the present disclosure reduces the temperature to which patient 82 is exposed from, for example, a predetermined temperature to a relaxation temperature that is 8 degrees Celsius lower than the predetermined temperature. The reduced relaxation temperature can be maintained for a similar time as the predetermined temperature is maintained, and then the temperature can be elevated to another temperature level that is lower than the predetermined temperature but higher than the relaxation temperature, thus initiating heat shock protein generation again. By continuously varying the temperature about which the temperature cycle is occurring, in essence, overlaying a short-term cycle on a long-term cycle, heat shock protein generation is maintained. The temperature range for application to patient 82 is a maximum of about 52 degrees Celsius to about 30 degrees Celsius. However, the temperature applied at 52 degrees Celsius is transitory to avoid damage to the skin of patient 82.

As can be seen in FIG. 122B, thermal frequency has two components. A first component 960 is a short-term frequency that can last from a few seconds to a maximum of five minutes. A second component 962 is a longer-term frequency that can be identified by averaging the short-term frequencies. Second component 962 has a longer frequency than first component 960, ranging from approximately half the frequency of first component 960 to approximately one tenth the frequency of first component 960.

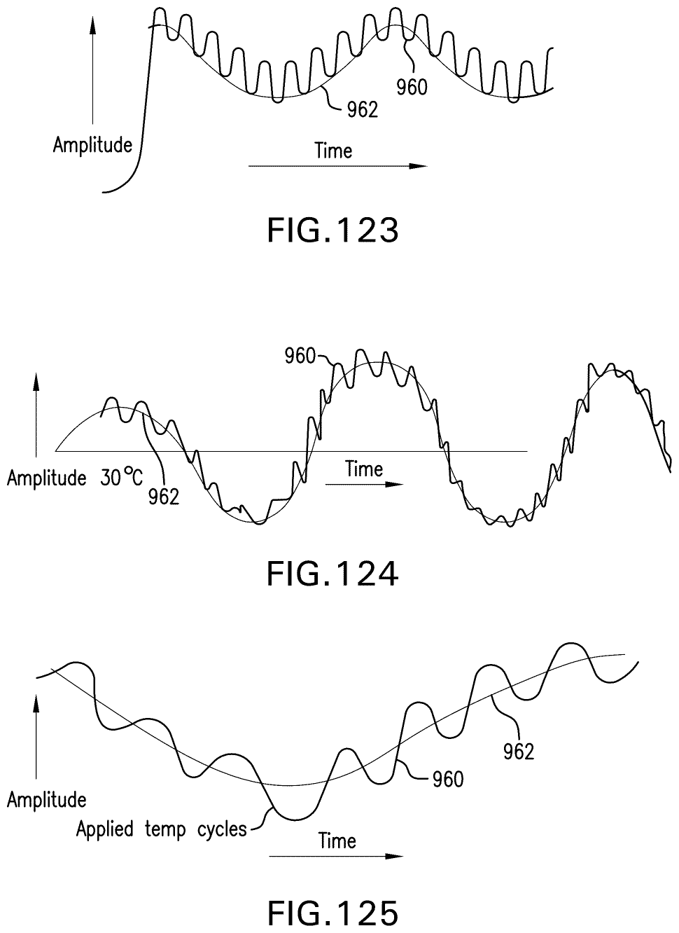

FIG. 123 shows another combination of first component 960 and second component 962. In FIG. 123, the short-term frequency, first component 960, is maintained at a constant amplitude, while the long-term frequency, second component 962, varies in amplitude so that the minimum and maximum temperature of the short-term frequency remains, for example, a fixed number of degrees, but that variation is about a varying long term change in amplitude. FIG. 124 shows another similar combination of first component 960 and second component 962, though the long-term frequency, second component 962, and amplitude are fixed, and the short-term frequency, first component 960, and amplitude are also fixed, but vary about the long-term frequency and amplitude. FIG. 125 shows a further variation, where the long-term frequency, second component 962, is much longer than the first component 960 frequency of FIGS. 123 and 124. Such longer frequency can sustain heat shock protein generation, though at a slightly reduced level than that shown in FIGS. 123 and 124.

Another factor to consider is the response of the body of patient 82. Raising the temperature of patient 82 can cause the body of patient 82 to respond in a “shock” fashion, causing reduced blood flow to internal organs and over or underheating of portions of the body. Accordingly, temperature rise rates of the body in response to applied heat, or to decreased heat, are preferably held within predetermined limits. To be effective, there is also a minimum thermal rise rate or decrease rate of the body of patient 82 in response to applied heat or reduced or decreased heat. The minimum rise rate is more than 0.01 degrees Celsius per minute and less than 0.30 degrees Celsius per minute. However, any narrower minimum range and maximum range are included in this temperature rise rate or temperature reduction rate in response to the application or removal of heat.

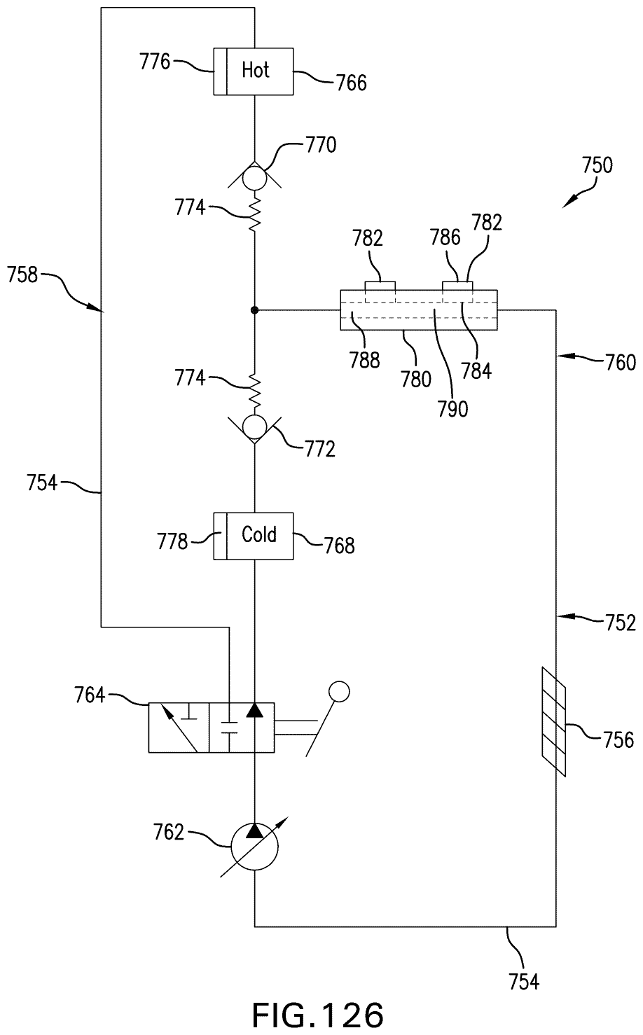

As described above, one challenge with applying temperature therapy to patient 82 is being able to induce high-frequency temperature cycles in patient 82. The issue is that temperature can be challenging to change, vary, or modify at frequencies approaching and/or above 1 Hz. A plurality of approaches accomplishes such variation. For example, two such systems are described herein using high-frequency valves to emit hot and cold air at high speeds toward ABTT 90 and potentially toward the veins that feed ABTT 90, shown in FIG. 138, and high-speed thermoelectric devices, such as those shown in FIGS. 139 and 140. Another such system for high-frequency temperature application is temperature therapy system 750 shown in FIGS. 126-130.

Temperature therapy system 750 includes a hydraulic or fluid flow circuit 752. Fluid flow circuit 752 includes a plurality of hydraulic lines 754 that connect the components of fluid flow circuit 752. Hydraulic lines 754 can be described as hydraulic hoses, hoses, hydraulic tubing, tubing, fluid flow passages, and the like. In a preferred embodiment, hydraulic lines 754 can be insulated to reduce heat loss or heating of cool fluid such as through radiation, convection, and conduction. Such insulation can be as simple as a wrap 756. In another embodiment, hydraulic lines 754 can be fabricated with integral insulation.

Fluid flow circuit 752 includes a temperature selection circuit 758 and an inductor circuit 760. Temperature selection circuit 758 receives water from pump 762, which can be considered a fluid driver or fluid supply for temperature selection circuit 758 and inductor circuit 760. Pump 762 can be a variable fluid pump or can be a fixed fluid pump with a simple on/off function. Fluid, which in a preferred embodiment is water, from pump 762 flows through temperature selection circuit 758 to inductor circuit 760, returning to pump 762. It should be noted that the fluid can include chemicals that reduce or eliminate the growth of bacteria, algae, and other organisms to minimize the maintenance requirements for fluid flow circuit 752. While water is a preferred fluid because of its heat capacity, other fluids designed to be heat transfer fluids for the removal of heat or addition of heat can be used.

Temperature selection circuit 758 includes a valve 764, an elevated temperature reservoir 766, a reduced temperature reservoir 768, and check valves 770 and 772. Check valves 770 and 772 can be biased to the closed position with a spring 774.

Valve 764 is electrically operated by a processor, such as any of the processors described herein, including those in the computers and controllers (control panels). Thus, the operating rate of valve 764 is controlled precisely, as will be described, to apply alternating temperatures to inductor circuit 760. In the embodiment of FIG. 126, valve 764 is a two-position valve that directs fluid toward elevated temperature reservoir 766 or toward reduced temperature reservoir 768. Fluid from elevated temperature reservoir 766 or reduced temperature reservoir 768 flows through respective check valve 770 or check valve 772 and then to inductor circuit 760.

While reservoir 766 is described as a reservoir, reservoir 766 can be, for example, a heater such as a Peltier or resistive heater that heats fluid on demand as it flows through the hydraulic lines 754 of temperature selection circuit 758. In an embodiment where reservoir 766 contains a volume of fluid, reservoir 766 can be heated by, for example, a resistive heater, a Peltier heater, a radiative heater, or other heater, labeled 776. In a preferred embodiment, reservoir 766 includes a volume of fluid. One advantage to a volume of fluid is that cool return fluid from reservoir 768 will have minimal effect on the temperature of reservoir 766.

Like reservoir 766, reservoir 768 can be a Peltier cooler, a chiller cooling by a refrigeration cycle, or other cooling device that cools fluid on demand as fluid flows through hydraulic lines 754. In an embodiment where reservoir 768 includes a volume of fluid, reservoir 768 can be cooled by, for example, a Peltier cooler, a refrigeration-type cooler, or other cooler 778.

In operation, valve 764 can be positioned as shown in FIG. 126 to select fluid flow to reduced temperature reservoir 768. The pressurized fluid then flows toward check valve 772, opening check valve 772. The fluid then flows toward inductor circuit 760. Pressurized fluid flowing through check valve 772 is prevented from flowing toward elevated temperature reservoir 766 by check valve 770.

Valve 764 can also be switched to enable fluid flow toward elevated temperature reservoir 766 instead of reduced temperature reservoir 768. Fluid flowing through reduced temperature reservoir 768 then causes check valve 770 to open, permitting fluid flow from elevated temperature reservoir 766 toward inductor circuit 760. Fluid is prevented from flowing toward reduced temperature reservoir 768 by check valve 772.

Fluid flow in inductor circuit 760 flows into inductor 780, returning to pump 762.

Pump 762 can be configured to supply fluid flow from, for example, 0 to 10 gallons per minute (GPM). In a configuration where each of reservoirs 766 and 768 contain a volume of fluid, the larger the volume the greater the stability of temperature flowing toward inductor circuit 760. Accordingly, each of reservoirs 766 and 768 can contain at least a half-gallon of fluid, but can contain several gallons of fluid that is either heated or cooled.

Absolute temperature control of reservoirs 766 and 768 is less important than the difference in temperature between reservoirs 766 and 768. Accordingly, a temperature differential of at least several degrees, such as at least three degrees Celsius, is a preferable minimum. Higher temperature differentials are preferred with higher flow rates to enable rapid temperature change or flux in inductor 780. For example, temperatures applied in cycles from 1 degree Celsius to 52 degrees Celsius, which is a temperature differential of 51 degrees Celsius, have been shown to elicit a therapeutic response in patients. It should be noted that temperatures applied to ABTT 90 above 44.5 degrees Celsius should be transitory rather than sustained because there is a possibility that damage to the skin or underlying tissue can result from such sustained temperatures.

Because of temperature hysteresis in the various components of temperature therapy system 750, the temperature flowing into inductor 780 can be much higher than 52 degrees Celsius since it requires some time for heat to flow through ABTT contact thermal inductor 782 to reach ABTT 90. Similarly, it can require a finite amount of time for heat to flow from ABTT contact thermal inductor 782 back into the fluid flowing through passage 790. The maximum and minimum temperatures permitted in fluid passage 790 are determined based on experimentation to avoid exceeding 52 degrees Celsius in contact with ABTT 90. Accordingly, elevated temperature reservoir 766 can be heated up to approximately 200 degrees fahrenheit, and reduced temperature reservoir 768 can be cooled to approximately 40 degrees fahrenheit. Each reservoir 766 and 768 are insulated in a preferred embodiment to reduce energy requirements for heating or cooling the fluid in the reservoir and, in at least the case of reservoir 766, for safety.

Inductor 780 is configured to include at least one, and preferably two, ABTT contact thermal inductors 782. Contact thermal inductors 782 include a first end 784 and a second end 786. First end 784 is preferably in direct contact with fluid 788 flowing in a fluid flow passage 790 formed in inductor 780. Second end 786 is configured to directly contact an ABTT 90 of a patient being treated by temperature therapy system 750.

The speed of thermal transfer is an important characteristic of thermal inductors 782. Accordingly, each thermal inductor 782 is fabricated from a material having a high thermal conductivity. For example, copper, silver, and/or diamond-based materials are all candidates for the material of thermal inductor 782. Indeed, diamond-based materials are preferred given that the conductivity of diamond is approximately five times greater than the thermal conductivity of copper.

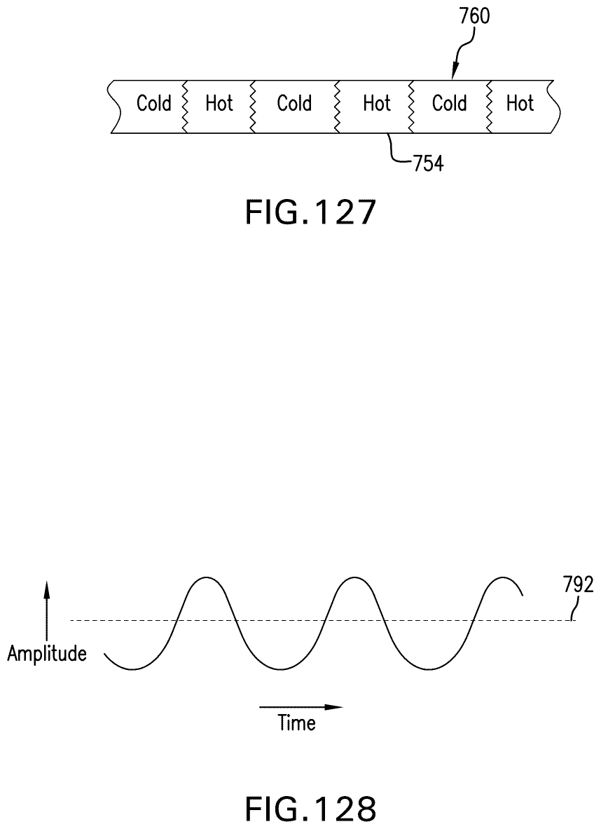

In operation, valve 764 is switched rapidly to alternate between the relatively high or elevated temperature of reservoir 766 and the relatively low temperature of reservoir 766. Referring to FIG. 127, the effect of this switching can be seen schematically. Because of the ability to alternate quickly between reservoirs 766 and 768, warmer and cooler fluid flows along inductor circuit 760 into inductor 780. In a preferred embodiment, fluid flow is laminar to reduce mixing between warmer and Cooler fluids. Since thermal inductors 782 each have high thermal conductivity, alternating temperatures are transmitted rapidly to each ABTT 90, enabling a relatively high alternating temperature cycle of one Hertz or more, enabling, for example, sustained generation of heat shock proteins and/or neutralizing undesired chemicals. Further, the temperature of each reservoir 766 and 768 can be varied with time to provide both short-term and long-term cycles, for example, such as those shown in FIGS. 122-125, that enable sustained generation of heat shock proteins.

Another technique for varying temperature is to vary the length of time that valve 764 is set to cause fluid to flow to reservoir 768 and to reservoir 766. As shown in FIG. 127, valve 764 alternates between hot and cold fluid flow evenly. The result is shown in FIG. 128, where the temperature applied to ABTT 90 varies uniformly about a baseline 792. However, because of thermal accommodation or thermal adaptation, patient 82's body adapts to such thermal variation and, after a period of 1-3 minutes, ceases responding to the variable input. Accordingly, the balance of temperature about baseline 792 needs to be adjusted to maintain a positive reactive response to the application of thermal therapy.

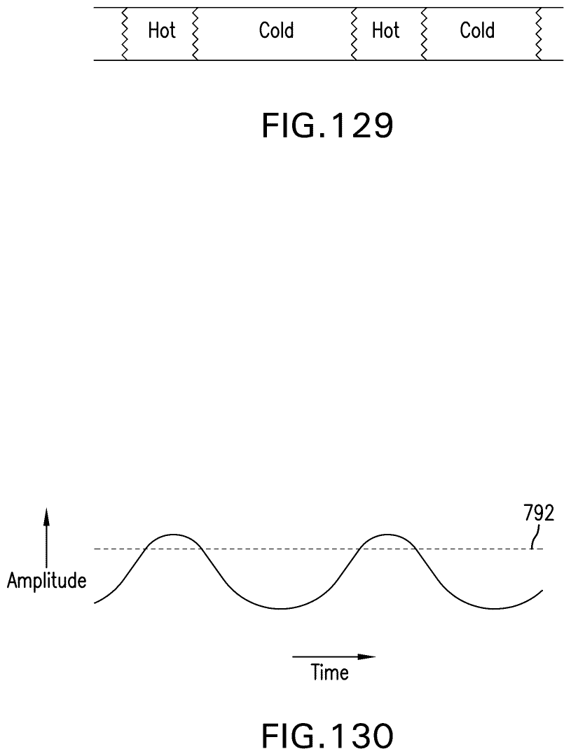

For example, as shown in FIGS. 129 and 130, the length of time that valve 764 is set to cause colder fluid to flow can be adjusted so that the length of time that colder fluid flows increases, as shown schematically in FIG. 129. The result can be seen by comparing FIG. 128 with FIG. 130. As shown in FIG. 130, the maximum temperature applied at ABTT 90 decreases, and the minimum temperature may also decrease, depending on the original velocity of the temperature applied in FIGS. 127 and 128. Additional adjustments can be accomplished by increasing the length of time that hot fluid flows and increasing or decreasing the temperature in reservoirs 766 and 768. The goal of these variations is to continuously change the effective temperature baseline 792 similar to what is shown in, for example, FIGS. 123 and 125. Such variations reduce the ability of patient 82's body to adapt to a specific temperature range, maintaining an elevated response to an applied temperature therapy protocol.

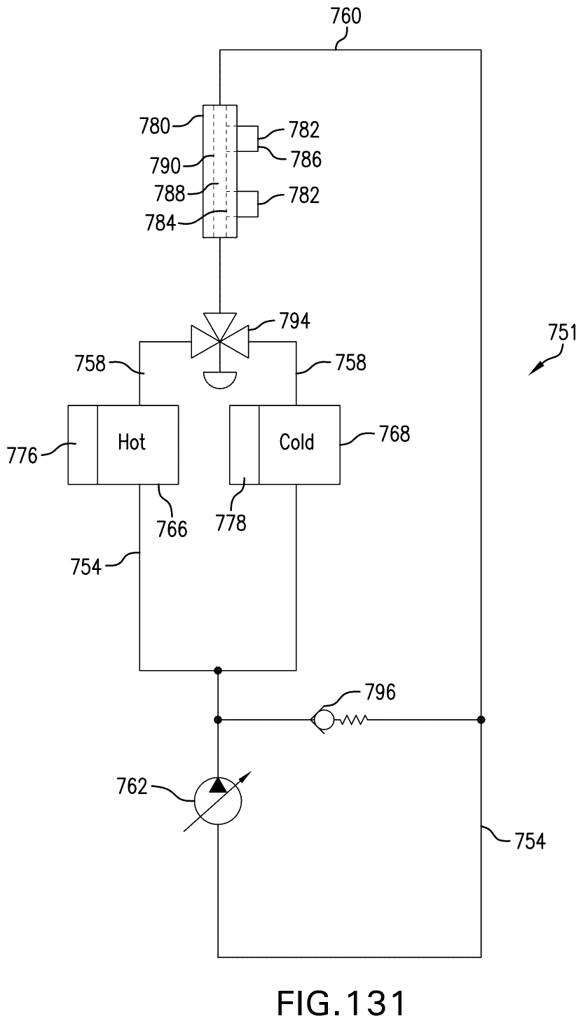

Another configuration that uses components of 126 is shown in FIG. 131. As with temperature therapy system 750 in FIG. 126, temperature therapy system 751 shown in FIG. 131 includes pump 762, reservoir 766, reservoir 768, and inductor 780. However, the temperature of the fluid to inductor 780 is controlled by a proportional valve 794. Proportional valve 794 functions by permitting cooled fluid from reservoir 766 to mix with heated fluid from reservoir 768. With this configuration, the available temperatures between minimum and maximum temperatures become nearly infinite. A pressure relief valve 796 can be provided to permit fluid to bypass reservoir 766 and reservoir 768 should proportional valve 794 be closed. However, under typical operating circumstances, pump 762 should be shut down if proportional valve 794 is not going to be actuated for a lengthy period. Alternatively, a large reservoir can be positioned along hydraulic lines 754 between the downstream side of pressure relief valve 796 and pump 762.

Applying heat to, and removing heat from, ABTTs 90 can be key to treating the various conditions herein. As such, many systems beyond those described hereinabove have been developed to provide heat and remove heat from ABTTs 90.

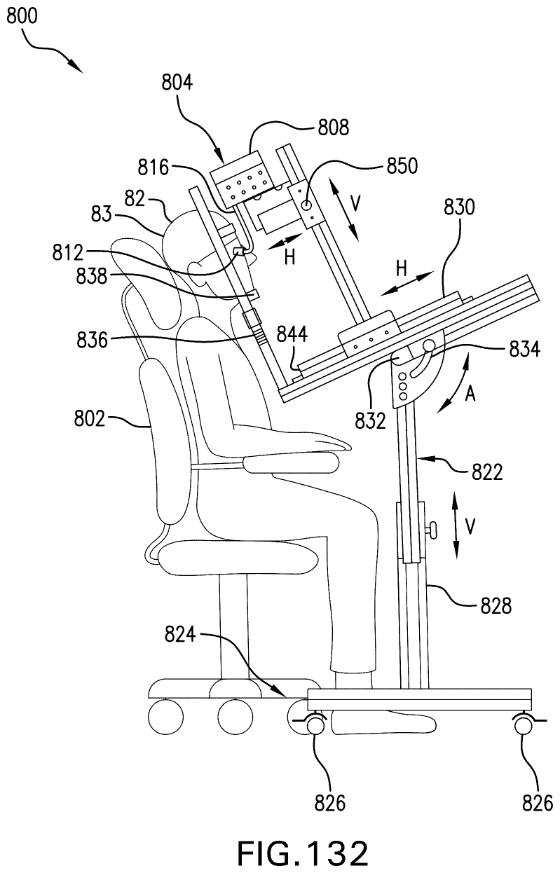

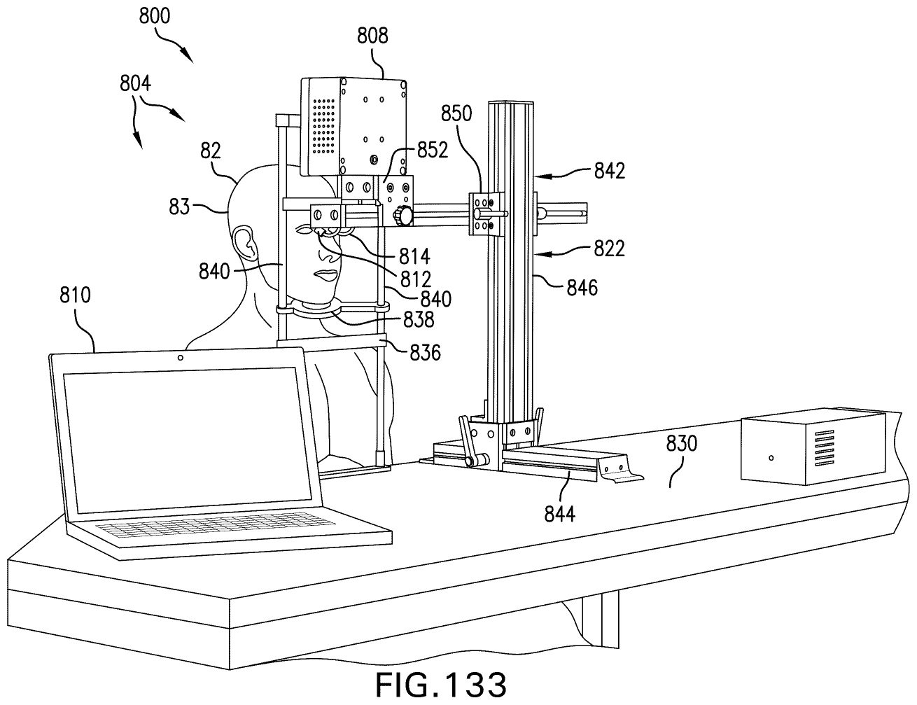

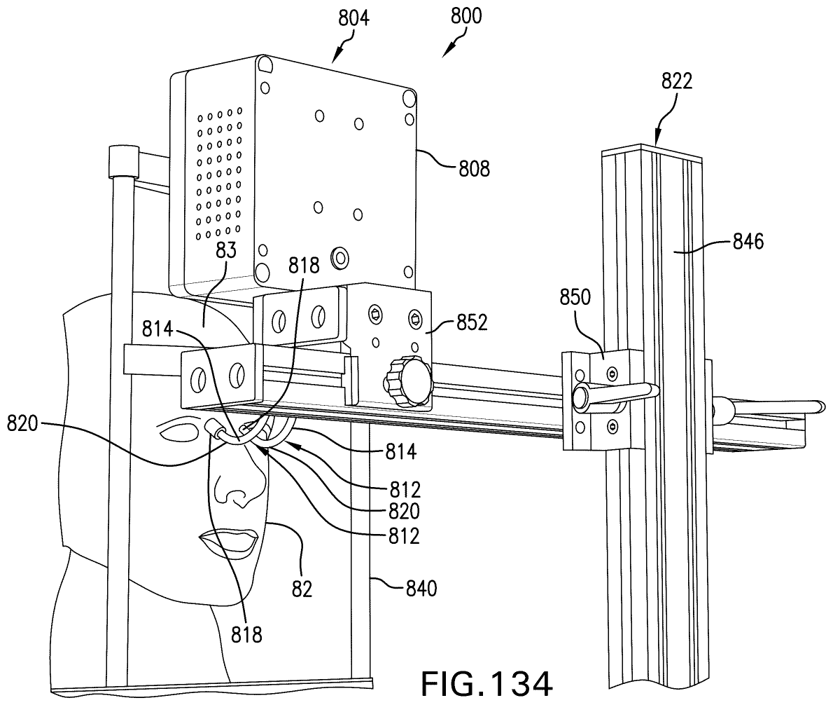

One such thermal therapy system 800 is shown in FIGS. 132-134. System 800 includes a patient support 802, which can be a chair. System 800 also includes an ABTT thermal interface system 804, which can be positioned on a support 806.

ABTT thermal interface system 800 includes a power supply 808, a controller 810, which can be a specialized controller or can be, for example, a laptop with custom-designed software, and ABTT interfaces 812. ABTT interfaces 812 are directly connected to power supply 808 and are, in the present embodiment, fully supported by power supply 808.

Controller 810 includes, as explained elsewhere herein, a processor and non-transitory computer-readable storage that is readable by the processor. The non-transitory computer-readable storage is configured to store the custom software used to control the functions of power supply 808. Controller 810 can be connected to power supply 808 by a cable or other electrical connection or can be connected to power supply 808 wirelessly.

ABTT interfaces 812 each includes an arm 814 that is attached to and supported directly by power supply 808 at a first, or proximal end 816 of arm 814. Each arm 814 includes a thermal device 818 at a second, or distal end 820. Thermal device 818 can be a heater, a cooler, or a combination heater/cooler. For example, thermal device 818 can be a Peltier cooler, or can be a Peltier cooler and heater. The wires to power thermal device 818 extend internally to arms 814, which are hollow tubes. Controller 810 controls power supply 808 to provide electrical power via the wires extending through arms 814 to thermal devices 818 to control both the temperature and amplitude of thermal devices 818. Because controller 810 controls the amplitude of the output of thermal devices 818, controller 810 also controls the frequency of cooling and/or heating, which as disclosed herein, is a valuable feature of providing thermal therapy. To be clear, the term “thermal device” includes both cooling and heating. In many applications, cooling is more important to the manipulation of the temperature of the hypothalamus than heating. Accordingly, the term “thermal” should be interpreted broadly.

Power supply 808 is supported on a power supply support system 822. Power supply support system 822 is configured principally to provide the ability to adjust the position of arms 814 and, more importantly, the position of thermal devices 818. Power supply support system 822 can include a floor interface 824, which can be a plurality of wheels 826 or legs (not shown). If floor interface 824 includes wheels 826, floor interface 824 can also include brakes on one or more wheels 826 to assist in maintaining the position of power supply support system 822.

Extending from floor interface 824 can be a vertically-extending frame 828. Vertically-extending frame 828 can include multiple pieces that connect to each other movable to enable vertical movement of an upper end of vertically-extending frame 828.

Power supply support system 822 can further include a work table or work surface 830. Work surface 830 can be rotationally connected to vertically-extending frame 828 at a pivot 832. A bracket 834 can be directly attached to vertically-extending frame 828 to provide support for work surface 830 as it rotates about pivot 832. The ability to rotate work surface 830 about pivot 832 enables an additional degree of freedom in positioning thermal devices 818 with ABTTs 90 since the rotation enables a plurality of orientations of arms 814 with respect to ABTTs 90, thus enabling an orientation where thermal devices 818 extend approximately parallel to ABTTs 90.

Positioned on work surface 830 can be a patient support frame 836 that includes a chin rest 838. Chin rest 838 can help in maintaining a position of patient 82's head 83, which can be beneficial in maintaining a position of ABTTs 90 with respect to thermal devices 818. Chin rest 838 is slidable along rails 840 of patient support frame 836 to enable precise positioning of chin rest 838 with respect to patient head 83.

Power supply support system 822 can include an upper power supply structure 842. Upper power supply structure 842 includes a first rail 844 that is attached to work surface 830 and extends parallel to work surface 830. A vertically-extending second rail 846 extends perpendicular to and is slidably positioned on first rail 844. Second rail 846 is configured to move parallel to work surface 830. Second rail 846 is movable with respect to work surface 830 to enable adjusting a position of thermal devices 818 in a direction that is transverse to, meaning toward or away from, a position of patient 82. As with the other adjustments associated with power supply support system 822, the ability to move second rail 846 enables providing precise adjustments of the position of thermal devices 818 with respect to ABTTs 90.

Upper power supply structure 842 further includes a third rail 848 oriented to extend perpendicular to second rail 846. Third rail 848 is slidably attached to second rail 846 by way of a slider bracket 850 to enable movement of third rail 848 vertically along second rail 846. Movement of vertically extending frame 828 can provide a large or gross adjustment of the position of thermal devices 818. The movement of third rail 848, on which power supply 808 is positioned, provides a finer adjustment of the vertical position of power supply 808 and, accordingly, thermal devices 818 with respect to ABTTs 90.

Power supply 808 can be attached to third rail 849 by a second slider bracket 852. Second slider bracket 852 can include at least the ability to move along third rail 848, and may also include a further ability to move in a direction that is parallel to the direction that second rail 846 moves along first rail 844. Thus, second slider bracket 852 provides additional fine adjustability of the position of thermal devices 818 with respect to ABTTs 90.

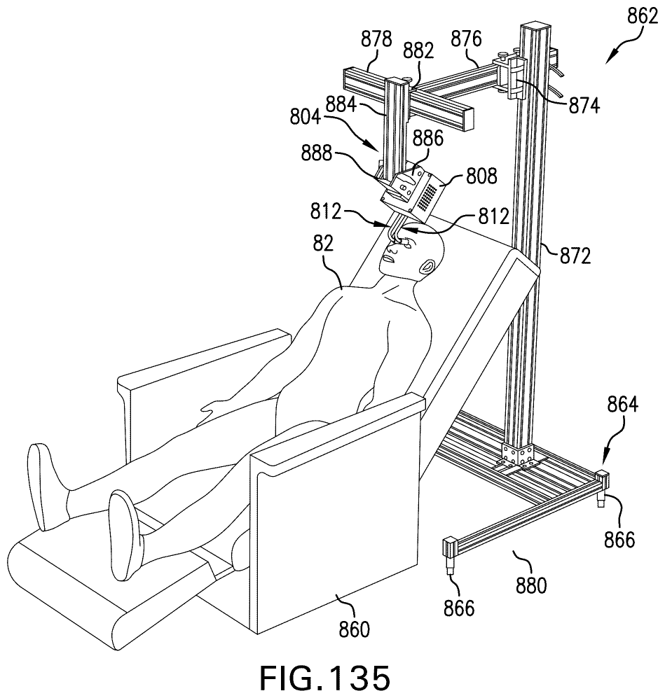

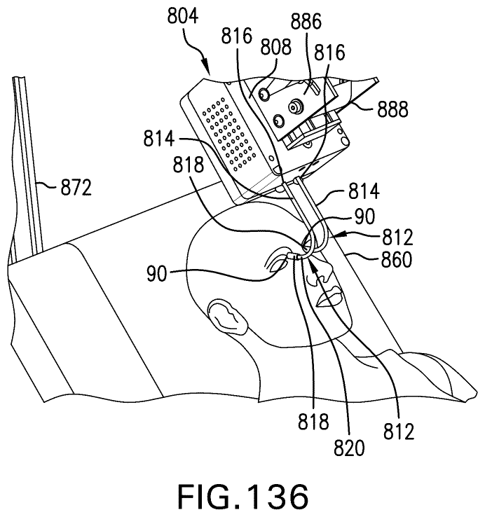

FIGS. 135 and 136 show another configuration of patient support 860 and power supply support 862 that is different from that shown in FIGS. 132-134. Patient support 860 is configured as a chair, such as a reclining chair. Power supply support 862 is free-standing, meaning that power supply support 862 is configured solely to support power supply 808. Elements of power supply 808 and ABTT interfaces 812 are similar to those in FIGS. 132-134. Accordingly, FIGS. 135 and 136 are labeled with similar numbers where the features are similar or are the same.

Power supply support 862 provides features similar to power supply support 822. As with power supply support 822, power supply support 862 is configured principally to provide the ability to adjust the position of arms 814 and, more importantly, the position of thermal devices 818. Power supply support 862 can include a floor interface 864, which can be a plurality of legs 866 or wheels (not shown).

Floor interface 864 can include a pair of horizontally-extending frames 868, spaced from each other by a base 870. Base 870 can be formed as a plurality of frames that provide support for other elements of power supply support 862.

Extending from base 870, and fixedly attached to base 870, can be a vertically-extending frame 872. A first slider bracket 874 can be movably attached to an upper end of vertically-extending frame 872. First slider bracket 874 moves along vertically-extending frame 872 to enable moving power supply 808 in a vertical direction, which thus adjusts a vertical position of thermal devices 818 for optimal interface with ABTTs 90.

A horizontally-extending first rail 876 extends perpendicular to and is slidably positioned on vertically-extending frame 872 by way of first slider bracket 874. First rail 876 can be slidable or movable with respect to first slider bracket 874 to provide movement with respect to vertically extending frame 872 in a direction perpendicular to vertically extending frame 872. As shown in FIG. 136, the movement of first rail 876 enables movement of power supply 808 and thermal devices 818 closer to and further away from patient or subject 82.

A second rail 878 oriented in a direction perpendicular to first rail 876 and approximately parallel to a ground surface 880 is fixedly attached to first rail 876. Attached to second rail 878 by way of a second slider bracket 882 is a vertically-extending third rail 884. Second slider bracket 882 is configured to slide along second rail 878 to provide transverse movement of third rail 884. Third rail 884 is configured to slide vertically with respect to second slider bracket 882 to enable vertical movement of power supply 808 and, accordingly, thermal devices 818.

A pivot bracket 886 is positioned at a lower end of third rail 884. Pivot bracket 886 is rotatable about a horizontally-extending pivot axis 888 that extends approximately parallel to second rail 878. Power supply 808 is directly attached to pivot bracket 886 and is movable with pivot bracket 886 to change the angular orientation of thermal devices 818 with respect to patient 82, particularly with respect to patient head 83 and ABTTs 90.

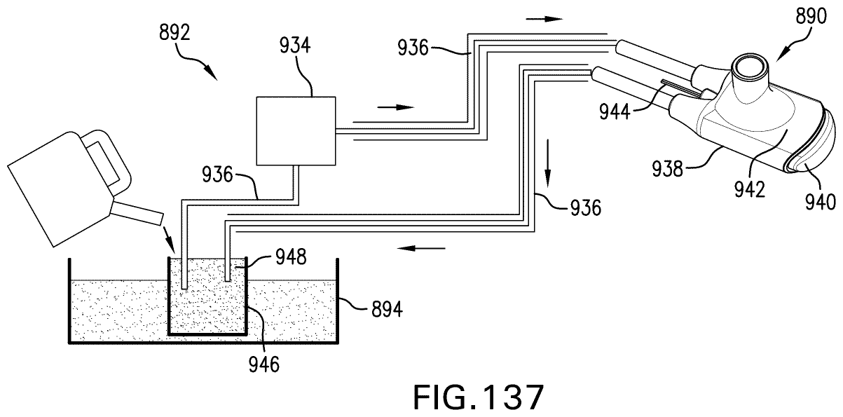

FIG. 137 shows a thermal device 890 in accordance with an exemplary embodiment of the present disclosure and a schematic view of a thermal system 892 connected to thermal device 890 to provide cooling fluid to thermal device 890. Thermal system 892 includes a fluid reservoir 894, a water pump 934 fluidly connected to the fluid in fluid reservoir 894 and to thermal device 890. The connections are by way of a plurality of insulated hoses 936. Thermal device 890 includes a body 938 that includes fluid channels for fluid to flow into and out from thermal device 890. The fluid in thermal device 890 flows through or adjacent to, including directly adjacent to a thermal interface 940. While thermal system 892 can conduct either heated or cooled fluid, in the embodiment of FIG. 137, thermal system 892 is principally for cooling.

Thermal interface 940 can be fabricated from a variety of materials. For example, ceramic, copper, silver, or diamond-based materials. While, for example, copper may be sufficiently conductive to obtain adequate thermal conductivity, for frequencies above one hertz a diamond-based material may be needed to provide faster thermal conductivity. Thermal interface 940 includes a curvilinear shape that can be cylindrical in a first direction and a second direction. However, the radius of thermal interface 940 in the first direction is much larger than the radius of thermal interface 940 in the second direction. Thus, thermal interface 940 in an end view appears to be somewhat flattened. This configuration enables ease of mating thermal interface 940 with ABTT 90 of patient 82, since ABTT 90 is in a region of the face that is curved.

Positioned within body 938 can be a temperature sensor 942. Temperature sensor 942 communicates with a processor, computer, or controller by way of a wire 944. Temperature sensor 942 provides feedback regarding the temperature of thermal interface 940.

In operation, water flows from fluid reservoir 894 into water pump 934 and then to thermal device 890 by way of the plurality of hoses 936. In thermal device 890, the water flows along or through thermal interface 940, cooling or heating thermal interface 940. Water returns to fluid reservoir 894 by way of another of the plurality of hoses 936.

Fluid reservoir 894 as presented in FIG. 137 can include a central reservoir 946 into which cold water 948 is poured. Central reservoir 946 is fluidly separated from outer fluid reservoir 894 so that fluid in central reservoir 946 does not mix with fluid in fluid reservoir 894. Fluid reservoir 894 may be filled with ice or a combination of cold water and ice. One advantage to this configuration is that no electricity is required to cool cold water 948 in central reservoir 946, thus simplifying the construction of fluid reservoir 894 and central reservoir 946.

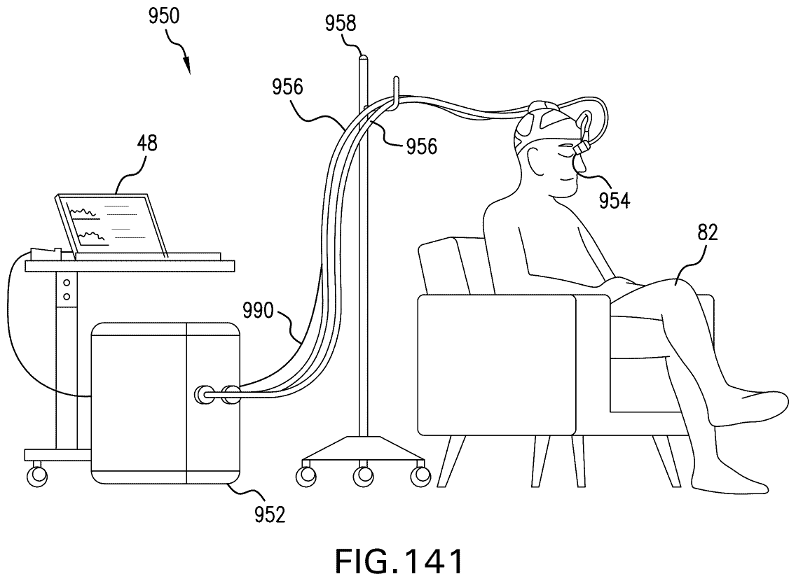

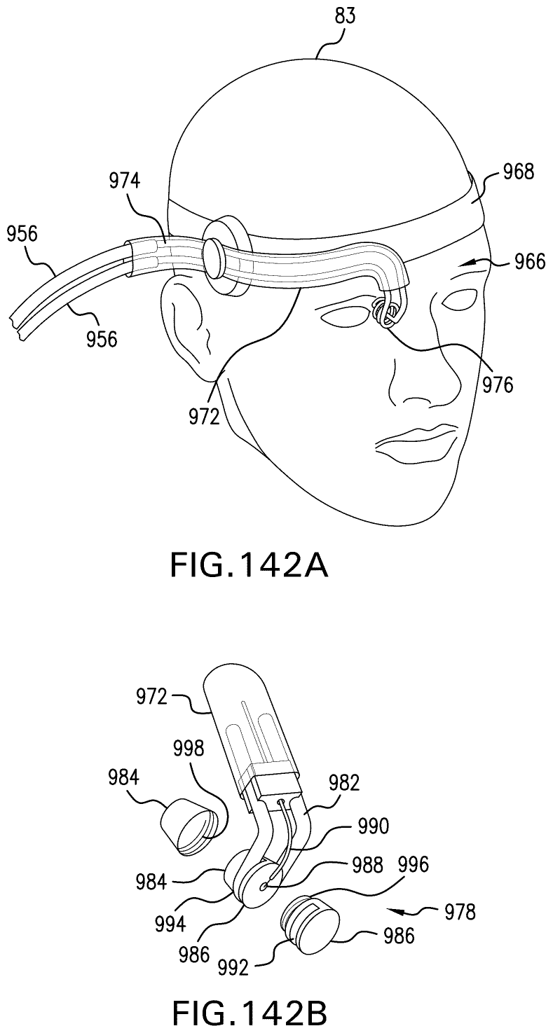

FIG. 141 shows another therapeutic thermal system 950. Therapeutic thermal system 950 includes a chiller cabinet 952, a head-mounted ABTT 90 interface 954, and fluid hoses 956 connecting chiller cabinet 952 to interface 954. A medical pole 958 can support fluid hoses 956 and one or more wires 964 extending to interface 954 to connect to valves within interface 954 or to a temperature sensor positioned within interface 954. Interface 954 can be based on thermal device 890 shown in FIG. 137, or the thermal devices shown in FIGS. 142-144. Therapeutic thermal system 950 can be controlled by computer 48 to control fluid flow from chiller cabinet 952 and the temperature of the fluid from chiller cabinet 952.

FIG. 142A shows a thermal device 966 in accordance with an exemplary embodiment of the present disclosure. Thermal device 966 is provided with fluid from, for example, chiller cabinet 952 by way of fluid hoses 956. Thermal device 966 is positioned on head 83 of patient 82 by way of, for example, a head support 968. In the embodiment of FIG. 142, head support 968 is configured as a headband. Thermal device 966 is attached to head support 968 by a support attachment 970, which assists in guiding fluid hoses 956 in a direction that is generally parallel to head support 968. Support attachment 970 can permit fluid hoses 956 to slide within head support 968 to enable the positioning of thermal device 966 to be in proximity to or direct contact with ABTT 90. Additionally, a rigid support 972 extends along head support 968. Rigid support 972 can be formed of an insulating material to minimize the discomfort of patient 82 when cool or warm fluid passes through fluid hoses 956.

Fluid hoses 956 can be directly connected to tube 974. Tube 974 can be formed of copper. Tube 974 can include a circular copper coil 976 that has a diameter of less than a half inch at the distal end to be compatible with ABTT 90 and the skin directly adjacent to ABTT 90. Thermal device 966 can include a temperature sensing device 980, such as a thermistor.

FIG. 142B shows another thermal device 978. Thermal device 978 includes a metal tube 982 and a thermally conductive metal piece, which may be described as a cap 984, and a cap attachment 986. Thermal device 978 also includes a thermistor 988. Thermistor 988 is connected by a wire 990 to, for example, computer 48. Cap attachment 986 includes a groove 992 which is configured to mate with metal tube 982 as metal tube 982 makes a circular bend 994 of about 180 degrees back on itself. Cap 984 and cap attachment 986 can be formed as a unitary piece, or may be two separate pieces, both configurations of which are shown in FIG. 142B. In the two-piece configuration, cap 984 attaches to cap attachment 986 by way of, for example, screw threads 996 formed on cap attachment 986 and mating screw threads 998 formed on cap 984.

In operation, fluid flows through circular copper coil 976, cooling or heating circular copper coil 976, and then applying temperature to ABTT 90 as described elsewhere herein. In FIG. 142B, fluid flows through metal tube 982, conducting heat to or removing heat from, i.e., cooling, cap 984 and cap attachment 986.

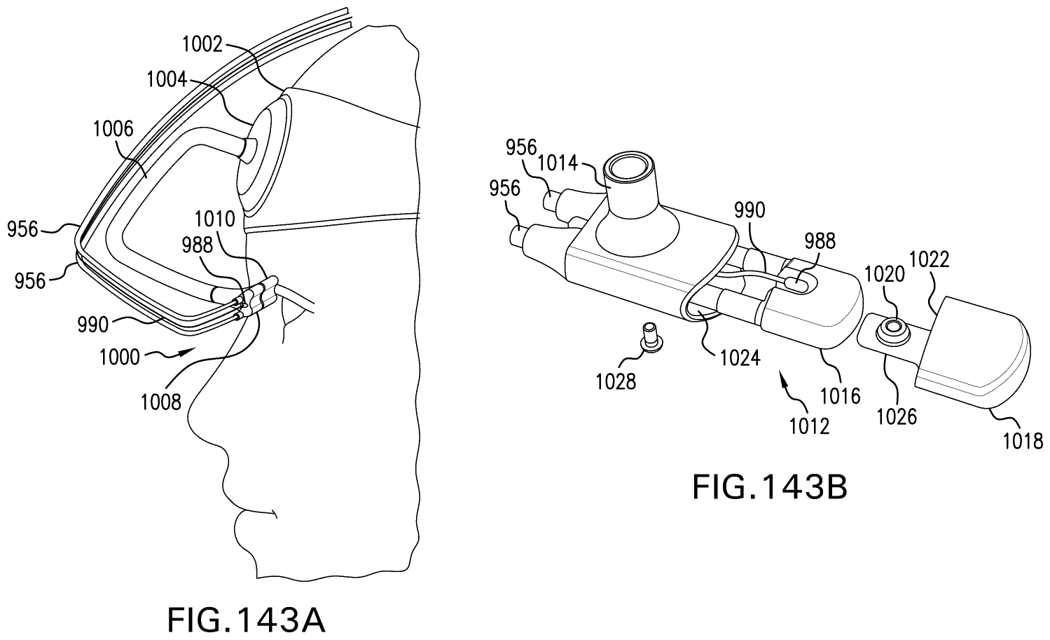

FIG. 143A shows another thermal device 1000 in accordance with an exemplary embodiment of the present disclosure. Thermal device 1000 is connected to, for example, chiller cabinet 952 by way of fluid hoses 956. Thermal device 1000 can include thermistor 988, which is connected to computer 48 by way of wire 990 or wirelessly. Thermal device 1000 is supported by a head-mounted support 1002, which can be, for example, a headband. Head-mounted support 1002 includes a base 1004 that is securely affixed to head-mounted support 1002. Base 1004 is formed of a rigid material. A support arm 1006 is rigidly attached to base 1004. Support arm 1006 is configured to be flexible, yet is sufficiently rigid to support thermal device 1000 in contact with ABTT 90. For example, support 1006 can have a flexible metal inner piece that is covered by a flexible plastic. An interface 1008 attaches to the distal end of support arm 1006. Thermal device 1000 includes a conductive heat transfer element 1010 that is directly attached to 1008. Conductive heat transfer element 1010 can include a fluid passage formed therein, or fluid hoses 956 can extend through interface 1008 and conductive heat transfer element 1010. In operation, heated and/or cooled fluid flows through fluid hoses 956 to transfer heat to or remove heat from conductive heat transfer element 1010 to heat or cool ABTT 90. As with other embodiments, conductive heat transfer element 1010 can be formed of copper, ceramic, silver, or diamond.

FIG. 143B shows a still further thermal device 1012 in accordance with an exemplary embodiment of the present disclosure. Still further thermal device 1012 includes a plastic body 1014, a reservoir 1016, which can be formed similar to a catheter balloon or a serum bag, and a conductive heat transfer element 1018. Fluid hoses 956 connect to reservoir 1016, which is shaped to fit within an interior 1024 of plastic body 1014. Conductive heat transfer element 1018 includes an interior 1022 into which reservoir 1016 extends. A tongue 1026 extends from a lower side of conductive heat transfer element 1018. When reservoir 1016 is positioned within interior 1024, conductive heat transfer element 1018 is positioned to cover interior 1024, with tongue 1026 extending directly between reservoir 1016 and a bottom side of plastic body 1014. Once conductive heat transfer element 1018 is pushed as far as possible into plastic body 1014, a fastener 1028 extends through a fastener opening (not shown) on the underside of plastic body 1014 into a fastener housing 1020 integrally formed with tongue 1026. In operation, fluid flows through fluid hoses 956 into reservoir 1016 transferring heat to or from conductive heat transfer element 1018, which transfers heat to or from ABTT 90 when conductive heat transfer element 1018 is in direct contact with ABTT 90.

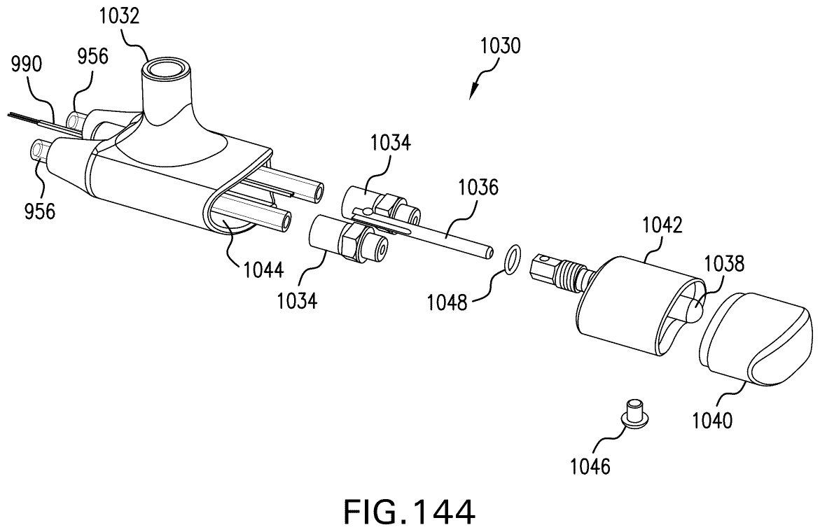

FIG. 144 shows yet another thermal device 1030 in accordance with an exemplary embodiment of the present disclosure. Thermal device 1030 includes a plastic body 1032 containing an internal cavity 1044. Positioned within internal cavity 1044 are hose connectors 1034, which connect to a thermistor capsule 1042. A thermally conductive heat transfer element 1040 is attached to thermistor capsule 1042. Such attachment can be, for example, by soldering or brazing. Thermistor 1038 is positioned internally to thermistor capsule 1042. A thermistor bushing 1036 can be positioned between thermistor 1038 and plastic body 1032. Thermistor bushing 1036 can be sealed with an O-ring 1048 to prevent leakage along thermistor 1038. A fastener 1046 can extend through a fastener opening (not shown) in plastic body 1032 to secure thermistor capsule 1042 and thermally conductive heat transfer element 1040 to plastic body 1032. In operation, fluid flows through fluid hoses 956 through hose connectors 1034 into thermistor capsule 1042 and thermally conductive heat transfer element 1040 to transfer heat to and or from ABTT 90 when thermally conductive heat transfer element 1040 is in direct contact with ABTT 90.

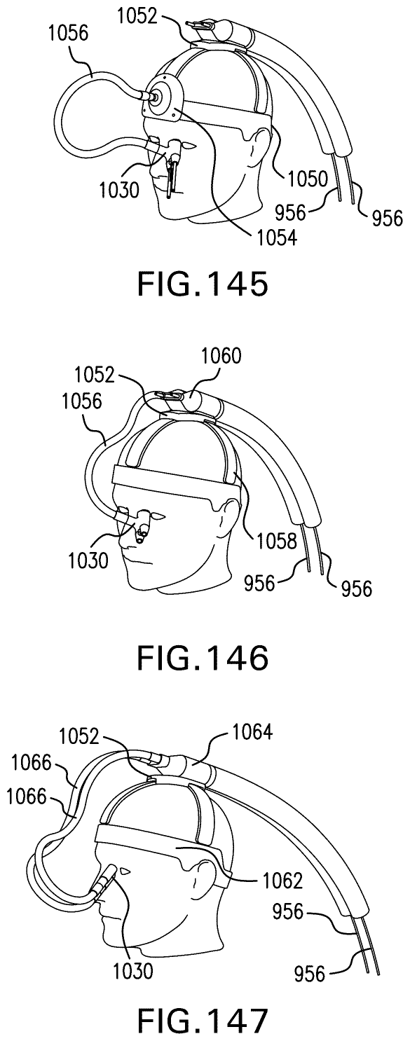

FIG. 145 shows a headband 1050 configured to support a thermal device in accordance with an exemplary embodiment of the present disclosure. Headband 1050 includes a top-mounted hose and wire support 1052 to which fluid hoses 956 and wire 990 can be secured. A base 1054 is rigidly attached to headband 1050. Base 1054 supports a gooseneck-type flexible arm 1056 that supports thermal device 1030 or any other thermal device shown herein.

FIG. 146 shows a further headband 1058 configured to support a thermal device in accordance with an exemplary embodiment of the present disclosure. Further headband 1058 is similar in some respects to headband 1050. However, a top support 1060 attaches to wire support 1052 of further headband 1058 that provides support for gooseneck-type flexible arm 1056.

FIG. 147 shows a still further headband 1062 configured to support a thermal device 1030 in accordance with an exemplary embodiment of the present disclosure. Still further, headband 1062 is similar to headband 1050 and headband 1058. However, top support 1064 is configured to support two gooseneck-type arms 1066. Each of the two gooseneck-type arms 1066 supports one fluid hose 956 in connecting to thermal device 1030. One of the two gooseneck-type arms 1066 can also support wire 990, or wire 990 can separately extend from top support 1064 to thermal device 1030, though wire 990 can be secured to one of two gooseneck-type arms 1066.

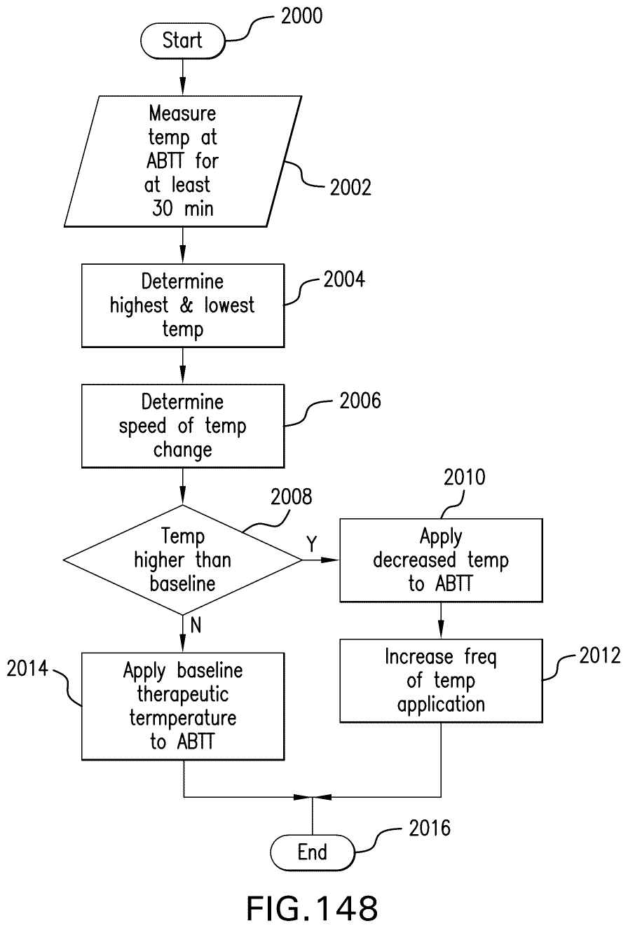

One aspect of treating diseases and conditions is understanding the behavior of the patient's brain and body. Referring to FIG. 148, a method for measuring and responding to a patient's temperature profile is presented. The method begins with a start process 2000. In start process 2000, registers can be cleared, communication protocols to one or more temperature sensors placed on subject or patient 82 can be established, and other activities needed to obtain and analyze temperature from subject or patient 82 can be initiated. Control then passes to a temperature measurement process 2002.

In temperature measurement process 2002, the temperature of the brain at the hypothalamus, which is what the temperature at ABTT 90 represents, is acquired. The temperature can be acquired for a predetermined minimum period. For example, if brain-guided hyperthermia is used for sleep modification, the predetermined minimum period can be 30 minutes. Because the temperature at the ABTT, representing the temperature of the hypothalamus, varies according to whole-body conditions, 30 minutes should be considered a minimum. For other conditions, the predetermined period can be hours to days. In unique situations, the period for temperature data acquisition can be many days. For this example, temperature is acquired at a single ABTT 90. In some situations, the temperature may need to be acquired at both ABTTs 90 so that a comparison of temperature changes can be made between the right and left sides.

It should be noted that the temperature signal at ABTT 90 is extremely noisy. Indeed, the signal varies with the pulse rate. Thus, the temperature signal can change as much as 200 times per minute or more. Traditional temperature measurement systems are unable to display temperature data that can change more than three times per second. Accordingly, the data must be analyzed at the signal level, which requires accessing the signal that represents temperature directly. By accessing the signal directly, transient temperature conditions can be identified that cannot be identified from a temperature display.

Once temperature signals at ABTT 90 have been acquired for the minimum predetermined period, for example, 30 minutes in a situation where sleep modification is intended, control passes from temperature measurement process 2002 to a temperature limit identification process 2004.

In temperature limit identification process 2004, the highest and lowest temperatures during the predetermined period are identified. As disclosed herein, because the highest temperature and/or the lowest temperature may be transient, it is preferred, and perhaps even critical, that the temperature signal be analyzed directly rather than attempting to rely on an output that may have insufficient capability in responding to transients that occur at a single heartbeat. Once the highest and lowest temperatures in the predetermined period are identified, control passes from temperature limit identification process 2004 to temperature velocity process 2006.

Temperature velocity process 2006 measures the speed at which temperature signals change at ABTT 90. The velocity of temperature change, which is typically measured in increments of one-thousandth of a degree, can be important in understanding the underlying condition of the brain and body. For example, a relatively high increasing temperature velocity may be indicative of stress or an

underlying disease or illness. As will be seen, to modify the behavior of the brain and body the temperature behavior at the hypothalamus must be well understood. That behavior includes the minimum and maximum transient temperatures, the velocity of temperature changes, and the direction of temperature changes. Once the velocity of temperature changes and direction of velocity changes are determined, control passes from temperature velocity process 2006 to a temperature comparison decision process 2008.

In temperature comparison decision process 2008, a determination is made as to whether the temperature is higher than a baseline temperature for subject or patient 82. It should be noted that every patient or subject 82 will have a unique temperature baseline, and treatment will nearly always be more effective by acquiring that baseline temperature data. The baseline temperature can be acquired during temperature measurement process 2002. However, a longer-term baseline temperature measurement is likely to be more effective. Accordingly, multiple temperature measurements over time can be acquired, or continuous temperature measurements can be acquired to understand the behavior of the temperature baseline over a single day or multiple days. If the temperature acquired in process 2002 is higher than the baseline, control passes from temperature comparison decision process 2008 to a temperature adjustment process 2010.

In temperature adjustment process 2010, the temperature applied to ABTT 90 is adjusted. In the present example, the temperature is decreased slightly to encourage a transition to sleep because the ABTT 90 temperature is higher than the baseline. As an example, if the baseline therapeutic temperature to be applied to ABTT 90 is 12 degrees Celsius, if the temperature of patient 82 is 0.2 degrees higher than the baseline, the temperature applied to ABTT can be changed from a therapeutic level of 12 degrees C. to a therapeutic level of 10 degrees C. to compensate for the elevated ABTT 90 temperature. Once the therapeutic temperature is adjusted, the control passes to a temperature frequency process 2012.

In temperature frequency process 2012, the frequency of temperature application can be adjusted. It should be understood that the hypothalamus quickly responds to a constant temperature applied to ABTT 90. Indeed, such response occurs within seconds to under a second, depending on the temperature applied to ABTT 90 and the initial temperature of ABTT 90, after which the hypothalamus moves to an equilibrium state where the hypothalamus determines that no biological adjustments are necessary. Accordingly, to sustain a response from the hypothalamus, the temperature applied to ABTT 90 must be at one or more frequencies. Indeed, referring to FIGS. 122-125, the frequencies of temperature applied to ABTT 90 should have a short-term component and a longer-term component. For example, a short-term frequency can be one Hertz, but this frequency can be overlaid on a longer-term frequency of, for example, 0.01 Hertz. The reason for such a combination of frequencies is that the hypothalamus can recognize temperature patterns, and the hypothalamus will adapt to a constant temperature pattern quickly. Once the hypothalamus adjusts to a constant frequency of temperature application, the hypothalamus can no longer be encouraged to provide a therapeutic response with the application of temperature and frequency to ABTT 90. Accordingly, the temperature must be applied to the hypothalamus with a short-term frequency, but the baseline temperature needs to be varied over time to avoid having the hypothalamus accept the frequency of application as being a normal condition. Simply stated, the goal of changing frequencies is to trick the hypothalamus into sustaining a biological response to temperature input to ABTT 90.

It should be noted that the need for changing frequency is based on the thermal profile of subject or patient 82. In an extreme case where the temperature at ABTT 90 is above the baseline with an elevated or increasing velocity, the frequency of reduced temperature application to ABTT 90 may need to be increased, such as from 2 Hertz to 1.5 Hertz, or potentially higher. It should be noted that in some situations no increase in frequency is needed. Accordingly, control can quickly pass from temperature frequency process 2012 to an end process 2016.

Returning to temperature comparison decision process 2008, if the temperature is not higher than the baseline, then the control passes to baseline temperature application process 2014. In process 2014, the baseline or unadjusted therapeutic temperature at the unadjusted therapeutic frequency is applied to ABTT 90. As occurs in process 2012, the applied therapeutic frequency has two components, both of which are at the nominal or baseline frequencies. The two components are the short-term frequency and the long-term frequency, which trick or provoke the hypothalamus into a biological response that causes the generation of chemicals and or hormones in response to the thermal stimulus. In the present example, the hypothalamus is tricked into generating sleep-inducing chemicals, making subject 82 feel sleepy, leading to a deeper and more restful sleep. Once the baseline temperature application process 2014 is complete, control passes to end process 2016, which functions as described elsewhere herein.

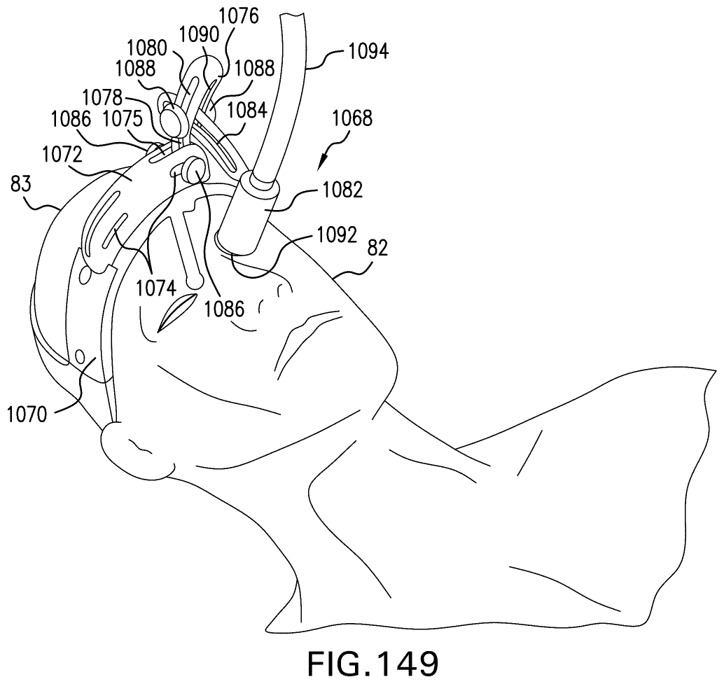

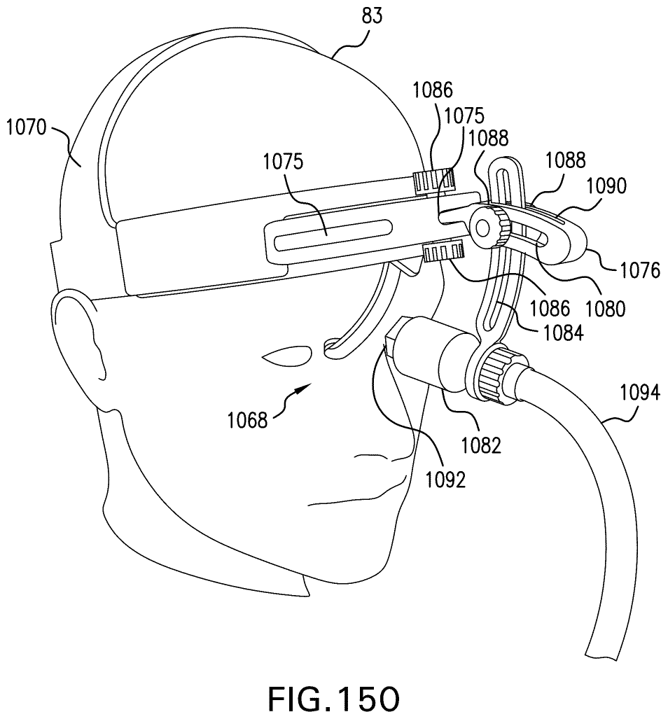

FIGS. 149 and 150 show a head-mounted thermal device 1068 in accordance with an exemplary embodiment of the present disclosure positioned on head 83 of patient 82. Head-mounted thermal device 1068 includes a support 1070 in the form of a headband. Support 1070 supports a rigid first support 1072 that includes at least one first slot 1074. A second support 1076 includes a second slot 1078 oriented in a first direction and a third slot 1080 oriented in a third direction that is perpendicular to second slot 1078. A third support 1082 includes a fourth slot 1084. At least one first slot 1074 intersects a first side slot 1075. Second slot 1078 of second support 1076 is inserted into first side slot 1075. A fastener 1086 extends into at least one first slot 1074 and through second slot 1078 to a far side of rigid first support 1072 to secure second support 1076 to rigid first support 1072. At least one first slot 1074 provides transverse adjustment of second support 1076 with respect to support 1070 and, accordingly head 83. Fourth slot 1084 extends into a second side slot 1090 formed in second support 1076. A fastener 1088 extends through third slot 1080 and fourth slot 1084 to secure third support 1082 to second support 1076. Each of third slot 1080 and fourth slot 1084 can be in the shape of a radius. The presence of third slot 1080, fastener 1088, and fourth slot 1084 enables multiple degrees of freedom in adjusting the position of ABTT interface 1092. ABTT interface 1092 can connect to a chiller by way of a cable support 1094, which can connect one or more fluid hoses 956 to the chiller. Cable support 1094 can also support wire 990 for a thermistor positioned within third support 1082. ABTT interface 1092 can alternatively include a thermoelectric device for transferring heat to or from ABTT 90.

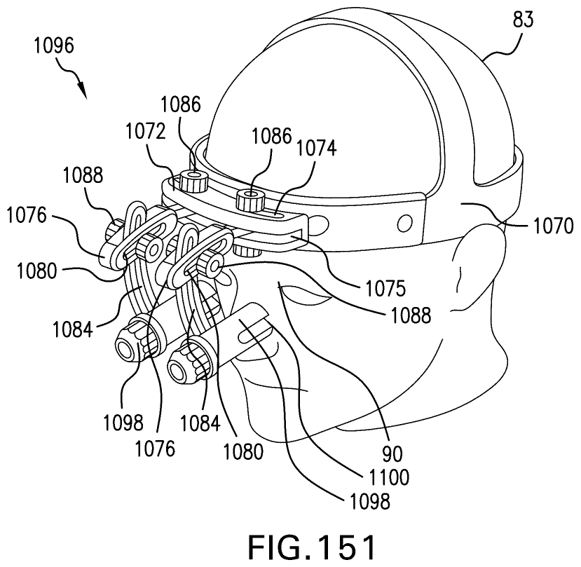

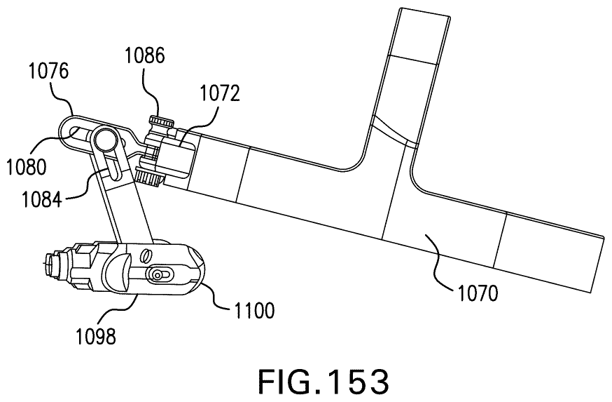

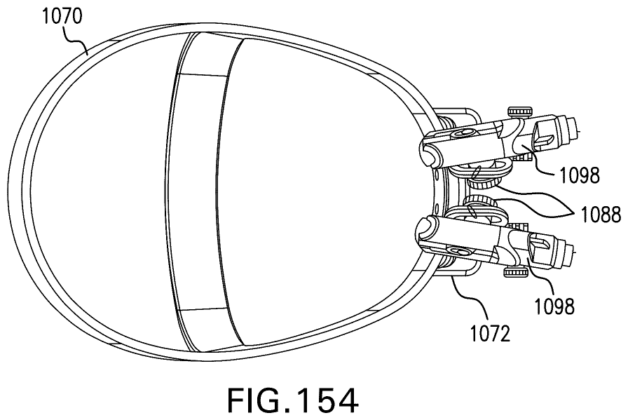

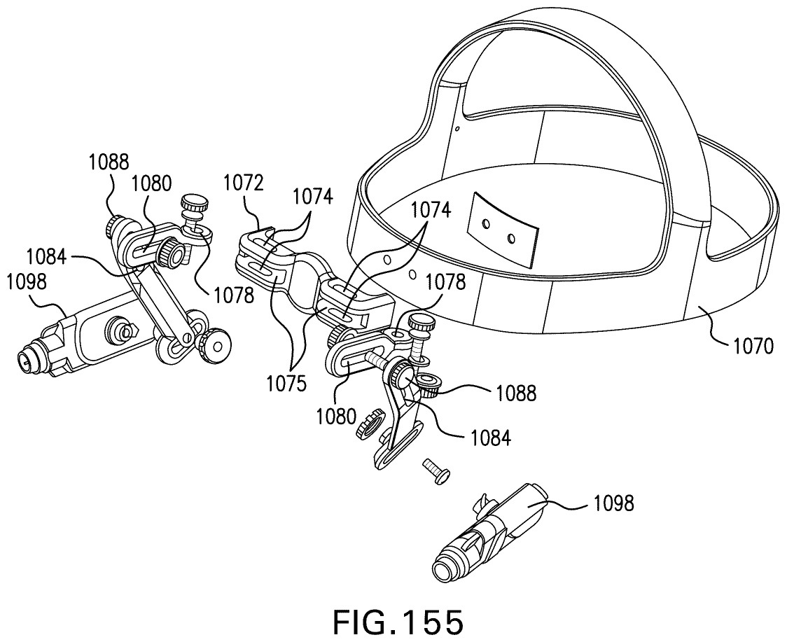

FIGS. 151-155 shows another head-mounted thermal device 1096 in accordance with an exemplary embodiment of the present disclosure. Head-mounted thermal device 1096 is similar to head-mounted thermal device 1068 and uses many similar components to head-mounted thermal device 1068. Accordingly, these components are numbered the same as those in FIGS. 149 and 150. FIGS. 151-155 include a pair of third supports 1098 that includes fourth slot 1084. Thus, rigid first support 1072, second supports 1076, and third supports 1098 provide multiple axes of adjustment to position an ABTT interface 1100 at ABTT 90.

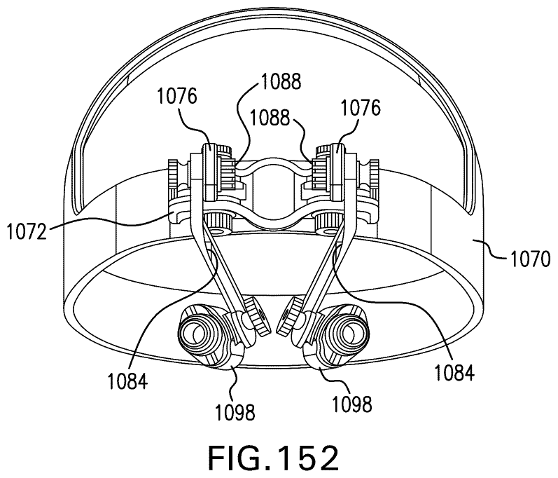

Each of third supports 1098 can receive a hose of fluid hoses 956 to transfer heat to and from ABTT interface 1100, or a thermoelectric device can be positioned on each of third supports 1098 to provide heat to or remove heat from ABTT interface 1100. While the configuration of FIGS. 152-155 contains some dimensional differences from the configuration of FIG. 151, the components function similarly to each other so components that function similarly are similarly numbered.

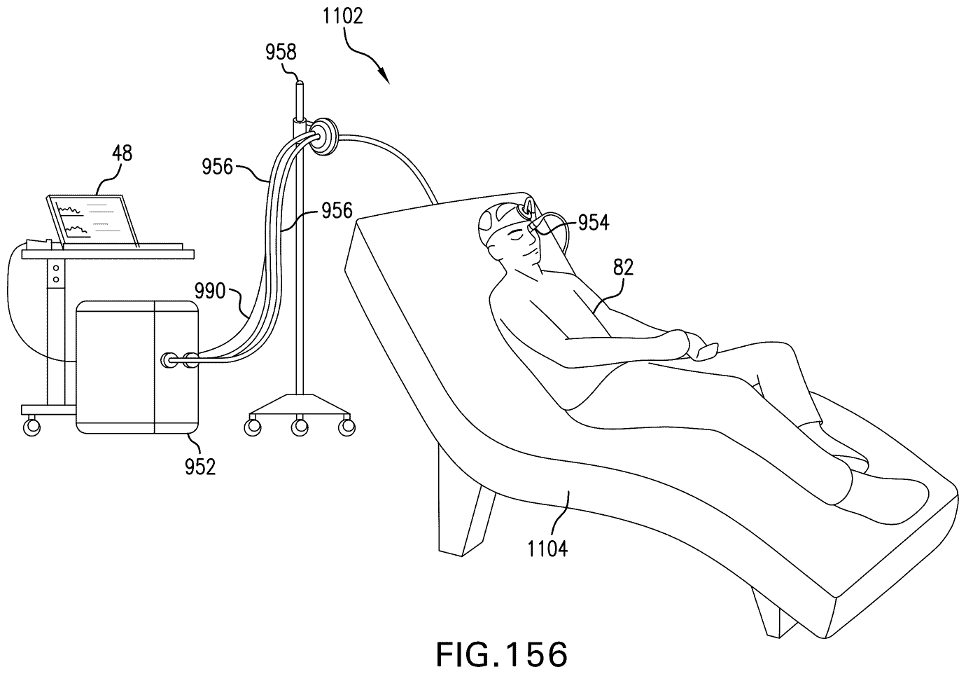

FIG. 156 shows a view of a thermal therapy system 1102 in accordance with an exemplary embodiment of the present disclosure. Thermal therapy system 1102 is similar to therapeutic thermal system 950 in FIG. 141. Accordingly, the elements are similarly numbered. In FIG. 141, patient 82 is positioned in a vertically-extending support, i.e., a chair. In FIG. 156, patient 82 is positioned in a reclining support 1104, similar to a couch or lounging-type chair.

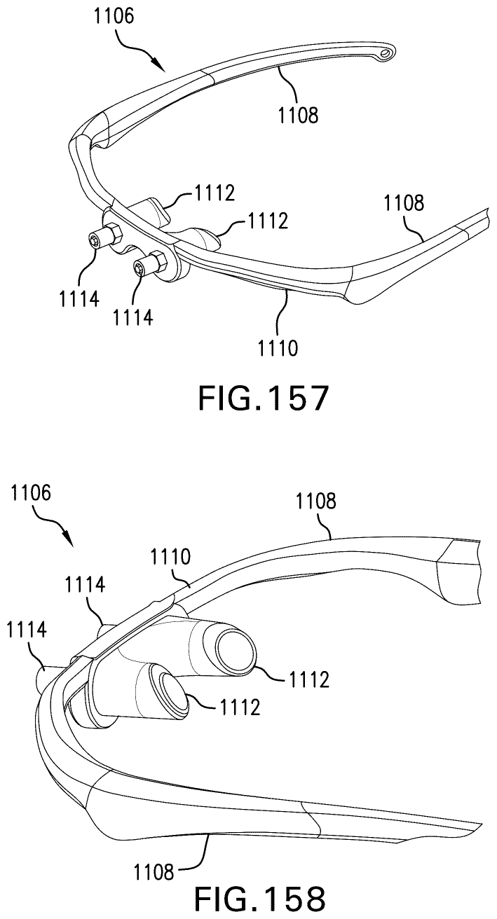

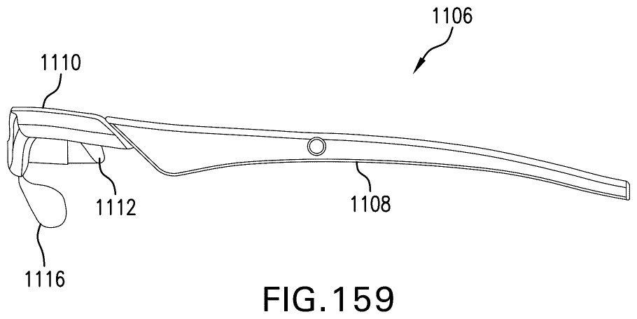

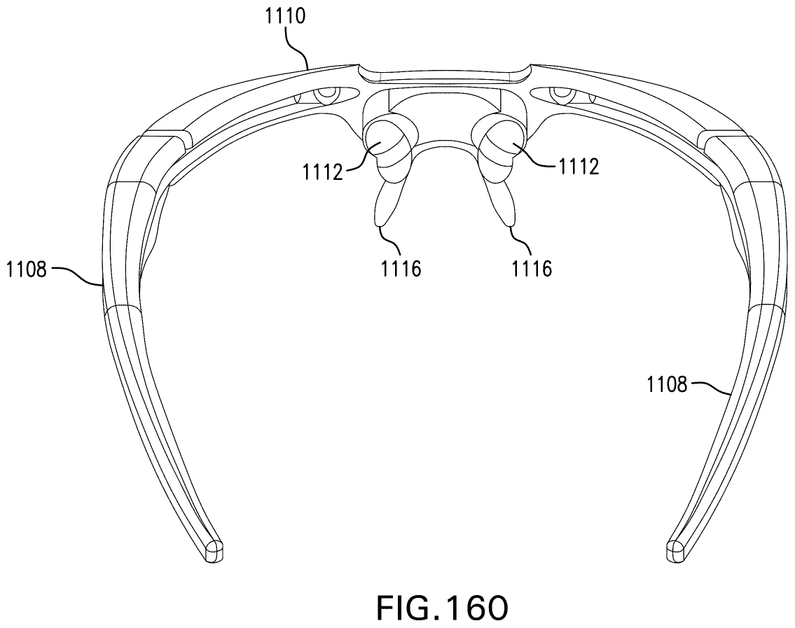

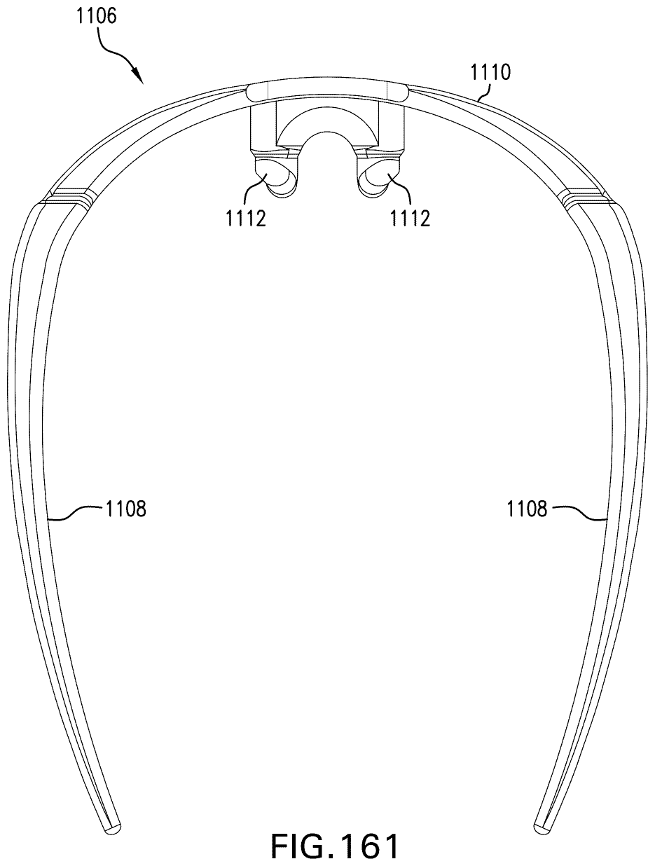

FIGS. 157-162 shows a perspective view of a head-mounted thermal device 1106 in accordance with an exemplary embodiment of the present disclosure. Head-mounted thermal device 1106 includes a pair of temples 1108 and a front frame 1110 that connects to each of temples 1108 and extends directly between temples 1108 to form a shape similar to eyeglass frames. Positioned on front frame 1110 is a pair of thermal interfaces 1112. Each of thermal interfaces 1112 is formed of a conductive material such as copper, ceramic, silver, or diamond. Each of thermal interfaces 1112 includes a fitting 1114 that is configured to mate with, for example, a respective one of fluid hoses 956. Each thermal interface 1112 terminates at an angle from horizontal that is approximately 45 degrees. However, the angle from horizontal is preferably in the range from 10 degrees to 80 degrees, is more in the range of 15 degrees to 75 degrees, is even more preferably in the range of 30 degrees to 60 degrees, and is most preferably in the range of 40 degrees to 50 degrees. The optimal 45-degree angle enables thermal interfaces 1112 to be aligned with ABTT 90 when head-mounted thermal device 1106 is worn by patient 82.

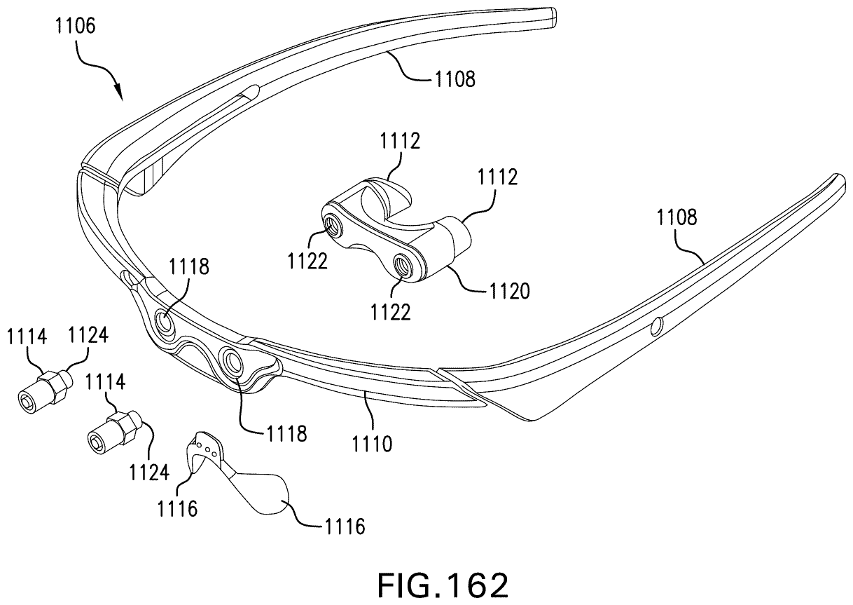

As shown in, for example, FIG. 162, head-mounted thermal device 1106 includes a nose piece 1116 that helps to support head-mounted thermal device 1106 on head 83 of patient 82. FIG. 162 also shows how head-mounted thermal device 1106 is assembled. Temples 1108 are attached to front frame 1110. Such attachment can be by pivots such as exist in eyeglasses. Each fitting 1114 extends through openings 1118 formed in front frame 1110 to engage with body 1120 from which each of thermal interfaces 1112 extends. Body 1120 can include screw threads 1122 that mate with screw threads 1124 formed on each fitting 1114.

In operating, fluid flows into a first fitting 1114 and through an internal passage formed in body 1120. The fluid applies heat to or removes heat from thermal interfaces 1112, which then applies heat to or removes heat from a respective ABTT 90.

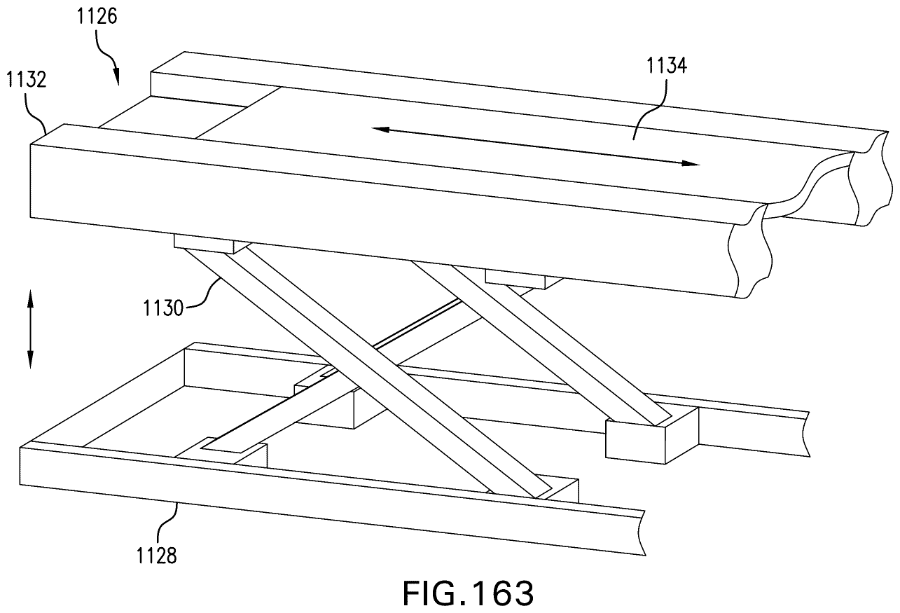

FIG. 163 shows a perspective view of a portion of a patient support configuration 1126 in accordance with an exemplary embodiment of the present disclosure. Patient support configuration 1126 can be used with any of the embodiments shown herein where the patient support is configured to be raised and lowered. Patient support configuration 1126 includes a base 1128, a scissor lift 1130 that is supported by base 1128, and a slide support 1132. Slide support 1132 supports a patient support 1134 that is slidable with respect to slide support 1132. Patient support 1134 enables patient 82 to be moved into or out of any of the chambers described herein.

While various embodiments of the disclosure have been shown and described, it should be understood that these embodiments are not limited thereto. The embodiments may be changed, modified, and further applied by those skilled in the art. Additionally, features of one embodiment may be used in another embodiment to the extent that embodiments are compatible. Further, elements of embodiments can be interchanged and combined to create new embodiments. Therefore, the embodiments are not limited to the details shown and described previously, but also include all such changes and modifications.

I claim:

1. A system for applying thermal therapy of a human being, the system comprising:
a chamber including an upper wall, two side walls extending longitudinally in a direction parallel to the upper wall, a rear end wall, and a front wall that form an interior;
a plurality of heaters positioned in the interior of the chamber;
at least one heater power supply connected to the plurality of heaters by a wire;
a processor connected to the at least one heater power supply to control an output of the at least one heater power supply to control heat output from the plurality of heaters according to at least one predetermined frequency, a predetermined maximum amplitude and a predetermined minimum amplitude; and
a support for the human, the interior of the chamber is sized and dimensioned to contain a body of the human positioned on the support.

2. The system of claim 1, wherein the front wall includes an opening sized and dimensioned for a human neck.

3. The system of claim 1, wherein the front wall is conformable to a human neck to be immediately adjacent to the human neck or in contact with the human neck.

4. The system of claim 1, wherein at least one temperature sensor configured to measure a temperature of the human body is attached to the chamber.

5. The system of claim 4, wherein the at least one temperature sensor is positioned on an articulating arm assembly.

6. The system of claim 4, wherein the at least one temperature sensor is directly connected to the front wall of the chamber.

7. The system of claim 1, wherein the support is vertically adjustable to raise the human body into the interior of the chamber and to lower the human body from the interior of the chamber.

8. The system of claim 1, wherein at least the two side walls of the chamber include a gas layer.

9. The system of claim 1, wherein the at least one predetermined frequency is a plurality of predetermined frequencies that are varied with time.

10. The system of claim 1, wherein the at least one predetermined frequency is at least 0.01 Hz.

11. A heater system for applying thermal therapy to an Abreu Brain Thermal Tunnel (ABTT) of a human, the heater system comprising:
an inductor sized and dimensioned to contact the ABTT; and
at least one of a cooler, a heater, or a combination cooler and heater connected to the inductor and configured to provide heat to or a reduction of temperature of the ABTT according to at least one first predetermined frequency, a predetermined first maximum amplitude, and a predetermined first minimum amplitude.

12. The heater system of claim 11, wherein the at least one first predetermined frequency is a plurality of frequencies that are varied with time.

13. The heater system of claim 11, wherein the at least one first predetermined frequency is at least 0.01 Hz.

14. The heater system of claim 11, wherein the inductor is positioned on the human located within an interior of a thermal chamber.

15. The heater system of claim 14, wherein the thermal chamber is configured to apply thermal cycles to the human of at least one second predetermined frequency, a predetermined second maximum amplitude, and a predetermined second minimum amplitude.

16. The heater system of claim 15, wherein the at least one first predetermined frequency is a different frequency from the at least one second predetermined frequency.

17. The heater system of claim 15, wherein the at least one second predetermined frequency is a plurality of second predetermined frequencies that change with time.

18. A heater system for applying thermal therapy to an Abreu Brain Thermal Tunnel (ABTT) of a human, the heater system comprising:
a first air reservoir configured to contain compressed air;
a first heater, cooler, or combination heater and cooler attached to the first air reservoir and configured to provide heating and/or cooling to the compressed air in the first air reservoir at a first temperature;
a first air nozzle attached to the first air reservoir, the first air nozzle having a first end and a second end;
a first valve attached to the first air reservoir at a location between the first air reservoir and the first end of the first air nozzle;
a second air reservoir configured to contain compressed air;
a second heater, cooler, or combination heater and cooler attached to the second air reservoir and configured to provide heating and/or cooling to the compressed air in the first air reservoir at a second temperature lower than the first temperature;
a second air nozzle attached to the second air reservoir, the second air nozzle having a first end and a second end; and
a second valve attached to the second air reservoir at a location between the second air reservoir and the first end of the second air nozzle, the first valve and the second valve are operated alternately for at least one predetermined frequency.

19. The heater system of claim 18, wherein the at least one predetermined frequency is at least 0.01 Hz.

20. The heater system of claim 18, wherein the at least one predetermined frequency is a plurality of predetermined frequencies that are varied with time.
